SEQUENCE LISTING (98 sequences)

SEQ ID NO: 1 - DNA, 369 bp, Artificial sequence
  Synthetic oligonucleotide
gaggttcacctggtggagtctgggggaggcctggtcaagcctggggggtccctgagactctcctgtgcagcctctggattcaccttcagtcgctatggcatgcactgggtccgccaggctccagggaaggggctggagtgggtctcatccattgttattaggagtgattacaaatattatgcagactcagtgaagggccgattcaccatctccagagacaacgccaagaattcactgtatctgcaaatgaacagcctcagagccgacgacacggccgtatattattgtgcgagagatctcgtggaaccgggtatcacaccagttggttgtgactactggggccggggaaccctggtcaccgtctcctca

SEQ ID NO: 2 - DNA, 324 bp, Artificial sequence
  Synthetic oligonucleotide
tccaatgagctgacccaggcaccctcggtgtcagtgtccccaggacagacggccagaatcacctgctttggagatgcattggcaaatcaatattcttattggtaccagcggaagccaggccaggcccctgtcttggtgatatataaagacagtgagaggccgtcagggatccctcagcgattctctgcctccaactcagggacaacagtcacgttgaccatcactggagtccaggcagaagacgaggctgactatttctgtcaatcggcagacagtactgggatctacaaggtgttcggcggtgggaccaagctgaccgtccta

SEQ ID NO: 3 - DNA, 390 bp, Artificial sequence
  Synthetic oligonucleotide
caggtgcagctaaaacagtggggcgcagggctgttgaagtcctcggagaccctgtccctcacgtgcgctttcaatggcaactccttcggtgctttctattggagttggatccggctgtccccagggaaggggctggagtggattggggaagtaaattatctaggaggtgccgactacaacccgtcgctcaagagtcgggtcaccatgtcggcagacacgtccaagaggcagttctccctgagcctcaagtctgtcaccgccgcggacaccggtgtctatttttgtgcgagaggtcggccccatgactactcgccggggagttattctcgccctcggcgttattacggtttggacgtctggggccaagggaccacggtcaccgtctcctca

SEQ ID NO: 4 - DNA, 324 bp, Artificial sequence
  Synthetic oligonucleotide
gagattgtgttgacgcagtctccagccaccctgtctttgtctccaggggacagcgccaccctctcctgcagggccagtcaggctgttagcaccacctacttagcctggtaccagcagacacgtggccaggctcccagactcctcatccatggtacatacaccagggccattggtatcccagacaagttcagtggcactgggtctgggacagacttcactctcaccatcagcggactggcgcctgaggattttgcagtgtattactgtcagcaatatagtagctcaccgtacacctttggccaggggaccaagattgagatcaca

SEQ ID NO: 5 - DNA, 363 bp, Artificial sequence
  Synthetic oligonucleotide
caggtgcaactggaagtgtctgggggaggcttggtcaagcctggagggtccctgagactctcctgtgcagcctctggattcaccttcactgactactacatgagttggatccgccaggctccagggaaggggctggagtgggttgcatacattagcggtataatgagttccacaaagtacgcagactctgtgaagggccgattcaccatctccagagacaacggcaagaactcagtgtatctgcaaatgaacagcctgacagccgaagacacggctgtctattactgtgcgagagagagagtagaaccgccatctgctgtcactgacttctggggccagggaaccctggtcaccgtctcctca

SEQ ID NO: 6 - DNA, 336 bp, Artificial sequence
  Synthetic oligonucleotide
cagtctgtgctgacgcagtcgccctcagtgtctggggccccagggcagagggtcaccatctcctgcactgggaccagctccaacatcggggcaggttatgatgtacactggtatcagcagtttcctggaacagcccccaaactcctcatctctcataacaccaatcggccctcaggggtccctgaccgattctctggctccaagtctggcacctcagcctccctggccatcactgggctccaggctgaggatgaggctgattattactgccagtcctttgacagcagcctgcggggttccagggtgttcggcggagggaccaagctgaccgtccta

SEQ ID NO: 7 - protein, 130 aa, Artificial sequence
  Synthetic antibody
QVQLKQWGAGLLKSSETLSLTCAFNGNSFGAFYWSWIRLSPGKGLEWIGEVNYLGGADYNPSLKSRVTMSADTSKRQFSLSLKSVTAADTGVYFCARGRPHDYSPGSYSRPRRYYGLDVWGQGTTVTVSS

SEQ ID NO: 8 - protein, 108 aa, Artificial sequence
  Synthetic antibody
EIVLTQSPATLSLSPGDSATLSCRASQAVSTTYLAWYQQTRGQAPRLLIHGTYTRAIGIPDKFSGTGSGTDFTLTISGLAPEDFAVYYCQQYSSSPYTFGQGTKIEIT

SEQ ID NO: 9 - protein, 123 aa, Artificial sequence
  Synthetic antibody
EVHLVESGGGLVKPGGSLRLSCAASGFTFSRYGMHWVRQAPGKGLEWVSSIVIRSDYKYYADSVKGRFTISRDNAKNSLYLQMNSLRADDTAVYYCARDLVEPGITPVGCDYWGRGTLVTVSS

SEQ ID NO: 10 - protein, 108 aa, Artificial sequence
  Synthetic antibody
SNELTQAPSVSVSPGQTARITCFGDALANQYSYWYQRKPGQAPVLVIYKDSERPSGIPQRFSASNSGTTVTLTITGVQAEDEADYFCQSADSTGIYKVFGGGTKLTVL

SEQ ID NO: 11 - protein, 121 aa, Artificial sequence
  Synthetic antibody
QVQLEVSGGGLVKPGGSLRLSCAASGFTFTDYYMSWIRQAPGKGLEWVAYISGIMSSTKYADSVKGRFTISRDNGKNSVYLQMNSLTAEDTAVYYCARERVEPPSAVTDFWGQGTLVTVSS

SEQ ID NO: 12 - protein, 112 aa, Artificial sequence
  Synthetic antibody
QSVLTQSPSVSGAPGQRVTISCTGTSSNIGAGYDVHWYQQFPGTAPKLLISHNTNRPSGVPDRFSGSKSGTSASLAITGLQAEDEADYYCQSFDSSLRGSRVFGGGTKLTVL

SEQ ID NO: 13 - protein, 8 aa, Artificial sequence
  Synthetic antibody
GNSFGAFY

SEQ ID NO: 14 - protein, 7 aa, Artificial sequence
  Synthetic antibody
VNYLGGA

SEQ ID NO: 15 - protein, 24 aa, Artificial sequence
  Synthetic antibody
ARGRPHDYSPGSYSRPRRYYGLDV

SEQ ID NO: 16 - protein, 8 aa, Artificial sequence
  Synthetic antibody
GFTFSRYG

SEQ ID NO: 17 - protein, 8 aa, Artificial sequence
  Synthetic antibody
IVIRSDYK

SEQ ID NO: 18 - protein, 16 aa, Artificial sequence
  Synthetic antibody
ARDLVEPGITPVGCDY

SEQ ID NO: 19 - protein, 8 aa, Artificial sequence
  Synthetic antibody
GFTFTDYY

SEQ ID NO: 20 - protein, 8 aa, Artificial sequence
  Synthetic antibody
ISGIMSST

SEQ ID NO: 21 - protein, 14 aa, Artificial sequence
  Synthetic antibody
ARERVEPPSAVTDF

SEQ ID NO: 22 - protein, 7 aa, Artificial sequence
  Synthetic antibody
QAVSTTY

SEQ ID NO: 23 - protein, 3 aa, Artificial sequence
  Synthetic antibody
GTY

SEQ ID NO: 24 - protein, 9 aa, Artificial sequence
  Synthetic antibody
QQYSSSPYT

SEQ ID NO: 25 - protein, 6 aa, Artificial sequence
  Synthetic antibody
ALANQY

SEQ ID NO: 26 - protein, 3 aa, Artificial sequence
  Synthetic antibody
KDS

SEQ ID NO: 27 - protein, 11 aa, Artificial sequence
  Synthetic antibody
QSADSTGIYKV

SEQ ID NO: 28 - protein, 9 aa, Artificial sequence
  Synthetic antibody
SSNIGAGYD

SEQ ID NO: 29 - protein, 3 aa, Artificial sequence
  Synthetic antibody
HNT

SEQ ID NO: 30 - protein, 12 aa, Artificial sequence
  Synthetic antibody
QSFDSSLRGSRV

SEQ ID NO: 31 - protein, 24 aa, Artificial sequence
  Synthetic antibody
CARGLNTMVRGVLNYYYYYGMDVW

SEQ ID NO: 32 - protein, 9 aa, Artificial sequence [sequence omitted]

SEQ ID NO: 33 - protein, 18 aa, Artificial sequence
  Synthetic antibody
CRATGTSPRASKDAFDIW

SEQ ID NO: 34 - protein, 16 aa, Artificial sequence
  Synthetic antibody
CARSVIGYYYYGMDVW

SEQ ID NO: 35 - protein, 15 aa, Artificial sequence
  Synthetic antibody
CARDQYGGNMYFQHW

SEQ ID NO: 36 - protein, 16 aa, Artificial sequence
  Synthetic polypeptide
CARSGRGHRGGSPDYW

SEQ ID NO: 37 - protein, 17 aa, Artificial sequence
  Synthetic polypeptide
CAKDWYLAMAGAAFDSW

SEQ ID NO: 38 - protein, 18 aa, Artificial sequence
  Synthetic polypeptide
CARDLLRNWDGHDAFDVW

SEQ ID NO: 39 - protein, 19 aa, Artificial sequence
  Synthetic polypeptide
CAKVEIHWWSNSLLGMDVW

SEQ ID NO: 40 - protein, 12 aa, Artificial sequence
  Synthetic polypeptide
CAKPVLSPFDYW

SEQ ID NO: 41 - protein, 26 aa, Artificial sequence
  Synthetic polypeptide
CTRDPSQYYDSRGHTTQTFTPSFDSW

SEQ ID NO: 42 - protein, 26 aa, Artificial sequence
  Synthetic polypeptide
CARGRPHDYSPGSYSRPRRYYGLDVW

SEQ ID NO: 43 - protein, 30 aa, Artificial sequence
  Synthetic polypeptide
CARDRVPSYSPSRRFSTKGAMWGKYGMDVW

SEQ ID NO: 44 - protein, 11 aa, Artificial sequence [sequence omitted]

SEQ ID NO: 45 - protein, 11 aa, Artificial sequence [sequence omitted]

SEQ ID NO: 46 - protein, 11 aa, Artificial sequence [sequence omitted]

SEQ ID NO: 47 - protein, 11 aa, Artificial sequence
  Synthetic polypeptide
CGADHGSGILF

SEQ ID NO: 48 - protein, 11 aa, Artificial sequence [sequence omitted]

SEQ ID NO: 49 - protein, 11 aa, Artificial sequence [sequence omitted]

SEQ ID NO: 50 - protein, 12 aa, Artificial sequence
  Synthetic polypeptide
CNSRDSSGKPSF

SEQ ID NO: 51 - protein, 12 aa, Artificial sequence
  Synthetic polypeptide
CSAWDDSLSGPV

SEQ ID NO: 52 - protein, 12 aa, Artificial sequence
  Synthetic polypeptide
CATLDINMTWVF

SEQ ID NO: 53 - protein, 11 aa, Artificial sequence
  Synthetic polypeptide
CLQHDTYPLTF

SEQ ID NO: 54 - protein, 12 aa, Artificial sequence
  Synthetic polypeptide
CCSYTSSSTEVF

SEQ ID NO: 55 - protein, 11 aa, Artificial sequence [sequence omitted]

SEQ ID NO: 56 - protein, 11 aa, Artificial sequence [sequence omitted]

SEQ ID NO: 57 - protein, 303 aa, Artificial sequence
  Synthetic polypeptide
VEQKTRPFTLPNLPLSSLSNSRAPLPISSMGISPDVBQSVQFQNGRCTLDGRLVGTTPVSLSHVAKIRGTSNGTVINLTELDGTPFHPFEGPAPIGFPDLGGCDWHINNTQFGHSFQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRL

SEQ ID NO: 58 - protein, 328 aa, Artificial sequence
  Synthetic polypeptide
VESKTKPFTLPILTISEMSNSRFPVPIDSLHTSPTENIVVQVQNGRVTLDGELMGTTQLLPSQICAFMGVLTRSTSRASDQADTATPRLFNYYWHIQLDNPNGTPYDPAEDIPGPLGTPDFRGKVFGVASQRNPDSTTRAHEAKVDTTAGRFTPKLGSLEISTESDDFHQNQPTRFTPVGIGVDNEADFQQWSLPDYSGQFTNMNMLAPAVAPNTPDEQLLFRSQLPSSGGRSNGILDCLVPQEWVQHFYQESAPSQTQVALVRYVNPDTGRVLFEAKLHKLGFMTIAKNGDSPITVPPNGYFRFESWVNPFYTLAPMGTFNGRRRIQ

SEQ ID NO: 59 - protein, 319 aa, Artificial sequence
  Synthetic polypeptide
VESRTKPFSVPVLTVEEMTNSRFPIPLEKLFTGPSSAFVVQPQNGRCTTDVLLGTTQLSPVNICTFRGDVTHITGSRNYTMNLASQNWNDYDPTEEIPAPLGTDDFVGKIQGVLTQTTRTDGSTRGKHATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDWVNQFYTLAPMDNGTFRRRAV

SEQ ID NO: 60 - protein, 330 aa, Artificial sequence
  Synthetic polypeptide
VESKTKPFTLPILTLGELSNSRFPAAIDMLYTDPNESIVVQPQNGRCTLDGTLQGTQLVPTQICAFRGTLISQTARAADSTDSPQRARNHPLHVQVKNLDGTQYDPTDDIPAVLGAIDFKGTVFGVASQRDVSGQQEQGHYATRAHEAHIDTTDPKYAPKLGTILIKSESNDFNTDQPIRFTPVGMGDNNWRQWELPDYSGRLTLNMNLAPAVSPSFPGERILFFRSIVPSAGGYGSGIDCLIQWEWVQHFYQEAAPSQSAVALVRYVNPDTGRNIFEAKLHREGFLTVANSGNNPIVVPPNGRFRFEAWVNQFYTLAPMGSGQGRRRAQ

SEQ ID NO: 61 - protein, 324 aa, Artificial sequence
  Synthetic polypeptide
VESKTKPGTLPILTISELTNSRFPIPIEQLYTAPNENNVVQCQNGRCTLDGELQGTTQLLSSAVCSYRGRTVANSGDNWDQNLLQLTYPSGASYDPTDEVPAPLGTQDFSGILYGVLTQDNVSEGTGEAKNAKGVYISTTSGKFTPKIGSIGLHSITENVHPNQQSRFTPVGVAQNENTPFQQWVLPHYAGALALNTNLAPAVAPTFPGEQLLFFRSRVPCVQGLRGQDAFIDCLLPQEWVNHFYQEAAPSQADVALIRYVNPDTGRTLFEAKLHRSSGFITVSHTGAYPLVVPPNGHFRFDSWVNQFYSLAPMGTGNGRRRVQ

SEQ ID NO: 62 - protein, 320 aa, Artificial sequence
  Synthetic polypeptide
VESKTKPFSLPILTLSELTNSRFPVPIDSLFTAQNNVLQVQCQNGRCTLDGELQGTTQLLPSGICAFRGRVATQINQRDRWHMQLQNLNGTTYDPTDDVPAPLGTPDFKGVVFGMVSQRNVGNDAPGSTRAQQAWVSTSPQFVPKLGSVNLRISDNDDFQFQPTKFTPVGVNDDDDGHPFRQWELPNYSGELTLNMNLAPPVAPNFPGEQLLFFRSFVPCSGGYNQFIIDCLIPQEWIQHFYQESAPSQSDVALIRYVNPDTGRTLFEAKLHRSGYITVAHSGDYPLVVPANGHFRFDSWVNQFYSLAPMGTGNGRRRAQ

SEQ ID NO: 63 - protein, 17 aa, Artificial sequence
  Synthetic peptide
CARDRLPASGSHWFHPW

SEQ ID NO: 64 - protein, 22 aa, Artificial sequence
  Synthetic peptide
CARMGRLELERRPHYYYPLDVW

SEQ ID NO: 65 - protein, 17 aa, Artificial sequence
  Synthetic peptide
CARATSQGATSYYFDSW

SEQ ID NO: 66 - protein, 19 aa, Artificial sequence
  Synthetic peptide
CAKVEIHYYSNSLLGMDVW

SEQ ID NO: 67 - protein, 17 aa, Artificial sequence
  Synthetic peptide
CARYNYYGSGSFVFDYW

SEQ ID NO: 68 - protein, 18 aa, Artificial sequence
  Synthetic peptide
CARDLLRNWGDHDAFDVW

SEQ ID NO: 69 - protein, 20 aa, Artificial sequence
  Synthetic peptide
CARDAQYCSGGRCYLVFDYW

SEQ ID NO: 70 - protein, 19 aa, Artificial sequence
  Synthetic peptide
CARGQMRTRGALFRRFDPW

SEQ ID NO: 71 - protein, 18 aa, Artificial sequence
  Synthetic peptide
CARDCRVGWVFTYGMDVW

SEQ ID NO: 72 - protein, 18 aa, Artificial sequence
  Synthetic peptide
CARYGAEYGSRSFYFLDW

SEQ ID NO: 73 - protein, 16 aa, Artificial sequence
  Synthetic peptide
CAREDCHGTSCYSGDW

SEQ ID NO: 74 - protein, 19 aa, Artificial sequence
  Synthetic peptide
CAKVRLTSYSIGWFSFDYW

SEQ ID NO: 75 - protein, 19 aa, Artificial sequence
  Synthetic peptide
CAKDFLRVYSYGWHSFDIW

SEQ ID NO: 76 - protein, 16 aa, Artificial sequence
  Synthetic peptide
CAKVTIIAAADLLDYW

SEQ ID NO: 77 - protein, 26 aa, Artificial sequence
  Synthetic peptide
CARVTGDCTGNRCSYWAYYYYGLDVW

SEQ ID NO: 78 - protein, 26 aa, Artificial sequence
  Synthetic peptide
CTRDPSQYYDSRGHYYQTFTPSFDSW

SEQ ID NO: 79 - protein, 30 aa, Artificial sequence; identical to SEQ ID NO: 43
  Synthetic peptide

SEQ ID NO: 80 - protein, 11 aa, Artificial sequence
  Synthetic peptide
CQQYYNSPLAF

SEQ ID NO: 81 - protein, 10 aa, Artificial sequence [sequence omitted]

SEQ ID NO: 82 - protein, 14 aa, Artificial sequence
  Synthetic peptide
CQSYDRSVSGSAVF

SEQ ID NO: 83 - protein, 12 aa, Artificial sequence
  Synthetic peptide
CATLDINMTWVF

SEQ ID NO: 84 - protein, 13 aa, Artificial sequence [sequence omitted]

SEQ ID NO: 85 - protein, 13 aa, Artificial sequence [sequence omitted]

SEQ ID NO: 86 - protein, 11 aa, Artificial sequence
  Synthetic peptide
CQSYDNRLRVF

SEQ ID NO: 87 - protein, 13 aa, Artificial sequence
  Synthetic peptide
CQSVDTRGTYKVF

SEQ ID NO: 88 - protein, 13 aa, Artificial sequence
  Synthetic peptide
CSSYAGTYNCVVF

SEQ ID NO: 89 - protein, 14 aa, Artificial sequence
  Synthetic peptide
CQSYDSRLSSNVVF

SEQ ID NO: 90 - protein, 14 aa, Artificial sequence
  Synthetic peptide
CQSYDRSLSKSRVF

SEQ ID NO: 91 - protein, 13 aa, Artificial sequence
  Synthetic peptide
CGTWDTSLRACLF

SEQ ID NO: 92 - protein, 14 aa, Artificial sequence
  Synthetic peptide
CGTWDLSLTAGWVF

SEQ ID NO: 93 - protein, 14 aa, Artificial sequence [sequence omitted]

SEQ ID NO: 94 - protein, 14 aa, Artificial sequence
  Synthetic peptide
CGSYAGSTTSGYVF

SEQ ID NO: 95 - protein, 11 aa, Artificial sequence
  Synthetic peptide
CLQHDTYPLTF

SEQ ID NO: 96 - protein, 11 aa, Artificial sequence [sequence omitted]

SEQ ID NO: 97 - protein, 540 aa, Artificial sequence
  Synthetic polypeptide
MKMASSDASPSDGSTANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDPTIKLIAMLYTPLYANNAGDDVFTVSCRVLTRPSPDFDFIFLVPPTVESRTKPFTVPILTVEEMTNSRFPIPLEKLFTGPSGAFVVQPQNGRCTTDGVLLGTTQLSPVNICTFRGDVTHIAGTHDYTMNLASQNWNNYDPTEEIPAPLGTPDFVGKIQGVLTQTTRGDGSTRGHKATVSTGSVHFTPKLGSVQFTTDTNNDLETGQNTKFTPVGVVQDGNSAHQNEPQQWVLPNYSGRTGHNVHLAPAVAPTFPGEQLLFFRSTMPGCSGYPNMNLDCLLPQEWVLHFYQEAAPAQSDVALLRFVNPDTGRVLFECKLHKSGYVTVAHTGPHDLVIPPNGYFRFDSWVNQFYTLAPMGNGAGRRRAL

SEQ ID NO: 98 - protein, 540 aa, Artificial sequence
  Synthetic polypeptide
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLYANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

(19) United States
(12) Patent Application Publication
(10) Pub. No.: US 2023/0054956 A1
(43) Pub. Date: Feb. 23, 2023

(54) HUMAN MONOCLONAL ANTIBODIES THAT NEUTRALIZE PANDEMIC GII.4 NOROVIRUSES

(71) Applicant: Vanderbilt University, Nashville, TN (US)

(72) Inventor: James E. CROWE, Jr., Nashville, TN (US)

(21) Appl. No.: 17/807,773

(22) Filed: Jun. 20, 2022

Related U.S. Application Data
(60) Provisional application No. 63/213,065, filed on Jun. 21, 2021.

(51) Int. Cl. C07K 16/10; G01N 33/569

(52) U.S. Cl. CPC C07K 16/1009; G01N 33/56983; G01N 2333/085; G01N 2469/10; C07K 2317/76; C07K 2317/33; C07K 2317/21

(57) ABSTRACT

The present disclosure is directed to antibodies binding to and neutralizing norovirus and methods for use thereof.

### PRIORITY CLAIM

This application claims benefit of priority to U.S. Provisional Application Ser. No. 63/213,065, filed Jun. 21, 2021, the entire contents of which are hereby incorporated by reference.

### STATEMENT REGARDING FEDERALLY-FUNDED RESEARCH

This invention was made with government support under NIH Grant T32GM120011 awarded by the National Institutes of Health. The government has certain rights in the invention.

### INCORPORATION OF SEQUENCE LISTING

The sequence listing that is contained in the file named “VBLTP0317US_ST25.txt”, which is 57,344 bytes and was created on Jun. 20, 2022, is filed herewith by electronic submission and is incorporated by reference herein.

### BACKGROUND

### 1. Field of the Disclosure

The present disclosure relates generally to the fields of medicine, infectious disease, and immunology. More particular, the disclosure relates to human antibodies binding to human norovirus.

### 2. Background

Since the licensure and use of rotavirus vaccines, human noroviruses (HuNoV) have become the major etiologic agent of epidemic and sporadic acute gastroenteritis (Glass et al., 2009). The persistence of HuNoVs is attributed to many factors, such as a low infectious dose, extreme environmental viral stability, high levels of shedding, and prolonged shedding even after symptoms have resolved (Fields et al., 2013). According to the Centers for Disease Control and Prevention, HuNoVs cause on average 19 to 21 million cases of infection and between 570 to 800 deaths in children under the age of five each year in the United States. HuNoVs infect people of all ages and, even though infection is characteristically acute and self-limiting, disease can become life threatening in children, the elderly, and the immunocompromised (Bok and Green, 2012). The correlates of HuNoV immunity in humans are poorly incompletely understood. There are several correlates of protection that have been described; the new capacity to perform in vitro neutralization testing described here may provide an improved correlate. Antibodies are clearly important to human immunity (Atmar et al., 2018).

One of the challenges for developing antibodies or vaccines to prevent HuNoV-associated disease is the extreme antigenic diversity of field strains. HuNoVs currently are classified phylogenetically into 7 different genogroups (GI-GVII) and at least 41 different genotypes (Vinjé, 2015). Viruses from genogroup I (GI) and the rapidly evolving genogroup II (GII) account for nearly all human infections. The HuNoV genome contains 3 open reading frames (ORF1, ORF2, and ORF3). ORF1 encodes nonstructural proteins, while ORF2 and ORF3 encode the major and minor capsid proteins, respectively. In the past, HuNoVs could not be cultivated in cell culture, but the VP1 and VP2 protein sequences could be expressed using a baculovirus expression system to produce HuNoV virus-like particles (VLPs) (Jiang et al., 1992). These VLP reagents have facilitated the study of HuNoV evolution, antigenicity and the emergence of new virus strains (Richardson et al., 2013; Erdman et al., 1989).

Since the mid-1990s, viruses from genogroup II genotype 4 (GII.4) have caused the majority of outbreaks, with new strains emerging every 2-3 years (Vinjé, 2015). In 2012, the GII.4 Sydney strain emerged and since then has continued to predominate among circulating strains. The molecular basis for antibody-mediated recognition of these strains and their mechanisms of action are not well characterized.

### SUMMARY

Thus, in accordance with the present disclosure, there is provided a method of detecting a norovirus infection in a subject comprising (a) contacting a sample from the subject with an antibody or antibody fragment having clone-paired heavy and light chain CDR sequences from Tables 3 and 4, respectively; and (b) detecting norovirus in the sample by binding of the antibody or antibody fragment to a norovirus antigen in the sample. The sample may be a body fluid, such as blood, sputum, tears, saliva, mucous or serum, semen, cervical or vaginal secretions, amniotic fluid, placental tissues, urine, exudate, transudate, tissue scrapings or feces. Detection may comprise ELISA, RIA, lateral flow assay or Western blot. The method may further comprise performing steps (a) and (b) a second time and determining a change in norovirus antigen levels as compared to the first assay.

The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

In another embodiment, there is provided a method of treating a subject infected with norovirus or reducing the likelihood of infection of a subject at risk of contracting norovirus, comprising delivering to the subject an antibody or antibody fragment having clone-paired heavy and light chain CDR sequences from Tables 3 and 4, respectively. The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

The antibody or antibody fragment may be administered prior to infection or after infection. The subject may be a pregnant female, a sexually active female, or a female undergoing fertility treatments. Delivering may comprise antibody or antibody fragment administration, or genetic delivery with an RNA or DNA sequence or vector encoding the antibody or antibody fragment.

In yet another embodiment, there is provided a monoclonal antibody, wherein the antibody or antibody fragment is characterized by clone-paired heavy and light chain CDR sequences from Tables 3 and 4, respectively. The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

In yet another embodiment, there is provided a hybridoma or engineered cell encoding an antibody or antibody fragment wherein the antibody or antibody fragment is characterized by clone-paired heavy and light chain CDR sequences from Tables 3 and 4, respectively. The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

In still yet another embodiment, there is provided a vaccine formulation comprising one or more antibodies or antibody fragments characterized by clone-paired heavy and light chain CDR sequences from Tables 3 and 4, respectively. The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

Also provided is vaccine formulation comprising one or more expression vectors encoding a first antibody or antibody fragment as defined above. The expression vector(s) may be Sindbis virus or VEE vector(s). The vaccine formulation may be formulated for delivery by needle injection, jet injection, or electroporation. The vaccine formulation may further comprise one or more expression vectors encoding for a second antibody or antibody fragment as defined above.

An additional embodiment includes a method of protecting the health of a placenta and/or fetus of a pregnant a subject infected with or at risk of infection with norovirus comprising delivering to the subject an antibody or antibody fragment having clone-paired heavy and light chain CDR sequences from Tables 3 and 4, respectively. The antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)2 fragment, or Fv fragment.

The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

The antibody or antibody fragment may be administered prior to infection or after infection. The subject may be a pregnant female, a sexually active female, or a female undergoing fertility treatments. Delivering comprises antibody or antibody fragment administration, or genetic delivery with an RNA or DNA sequence or vector encoding the antibody or antibody fragment. The antibody or antibody fragment may increase the size of the placenta as compared to an untreated control and/or may reduce viral load and/or pathology of the fetus as compared to an untreated control.

Yet another embodiment involves a method of determining the antigenic integrity, correct conformation and/or correct sequence of a norovirus antigen comprising (a) contacting a sample comprising the antigen with a first antibody or antibody fragment having clone-paired heavy and light chain CDR sequences from Tables 3 and 4, respectively; and (b) determining antigenic integrity, correct conformation and/or correct sequence of the antigen by detectable binding of the first antibody or antibody fragment to the antigen. The sample may comprise recombinantly produced antigen. The sample may comprise a vaccine formulation or vaccine production batch. Detection may comprise ELISA, RIA, western blot, a biosensor using surface plasmon resonance or biolayer interferometry, or flow cytometric staining.

The first antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the first antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The first antibody fragment may be a recombinant scFv (single chain fragment variable) antibody, Fab fragment, F(ab′)2 fragment, or Fv fragment. The method may further comprise performing steps (a) and (b) a second time to determine the antigenic stability of the antigen over time.

The method may further comprise (c) contacting a sample comprising the antigen with a second antibody or antibody fragment having clone-paired heavy and light chain CDR sequences from Tables 3 and 4, respectively; and (d) determining antigenic integrity of the antigen by detectable binding of the second antibody or antibody fragment to the antigen. The second antibody or antibody fragment may be encoded by clone-paired variable sequences as set forth in Table 1, may be encoded light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. Alternatively, the second antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The second antibody fragment may be a recombinant scFv (single chain fragment variable) antibody, Fab fragment, F(ab′)2 fragment, or Fv fragment. The method may further comprise performing steps (c) and (d) a second time to determine the antigenic stability of the antigen over time.

Also provided is a human monoclonal antibody or antibody fragment, or hybridoma or engineered cell producing the same, wherein the antibody or antibody fragment binds to norovirus capsid protein P domain and/or S domain. The human monoclonal antibody or antibody fragment may bind to norovirus capsid protein P domain P1 or P2 subdomain. The antibody or antibody fragment may cross-react with multiple norovirus GI and/or GII strains.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-C. Neutralization of GII.4 Sydney 2012 using virus-like particles (VLPs) or live virus. (FIG. 1A) Half-maximal effective concentrations (EC₅₀) for all isolated IgGs and IgAs using a VLP blockade assay, and the antibody concentration at which hemagglutination was inhibited when using VLPs and O+ red blood cells. > symbols indicate blockade EC₅₀ values >100 μg/mL for IgGs or >113 μg/mL for IgAs, or HAI titers >15 μg/mL. (FIG. 1B) Plotted are the absorbance values when plates were read at optical density (O.D.) 450 nm for selected IgGs and IgAs when antibodies were diluted serially, combined with GII.4 Sydney 2012 VLPs and added to porcine gastric mucin (PGM). (FIG. 1C) Inhibition of replication of GII.4 Sydney 2012 virus using selected IgGs, IgAs and a non-specific human monoclonal antibody (mAb) were tested in a human intestinal enteroid system. An additional control for each experiment was virus incubated without a mAb. The half-maximal inhibitory concentration (IC₅₀) for each mAb is indicated in each individual graph. The data presented is an average of two independent experiments for NORO-263, -250B, -320, -273A, -318 and a dengue virus-specific control antibody 2D22. The number of genome equivalents for each concentration tested for each mAb including the no antibody control was the average of 6 replicates tested.

FIG. 2. Competition binding of GII.4 specific mAbs on GII.4 Sydney 2012 P domain with the Octet® Red96 system. Epitope binning was performed using biolayer interferometry. GST-tagged GII.4 Sydney 2012 P domain was loaded onto anti-GST tips, then the first antibody was loaded followed by loading of the second antibody. The numerical data indicate percent binding of the second antibody in the presence of the first antibody. Yellow, green, and magenta boxes indicate potential binding groups.

FIG. 3. Characterization of human mAb binding to GII.4 Syndey VLPs. Reactivity of serially diluted mAbs or a no-antibody control for GII.4 Sydney 2012 test by ELISA.

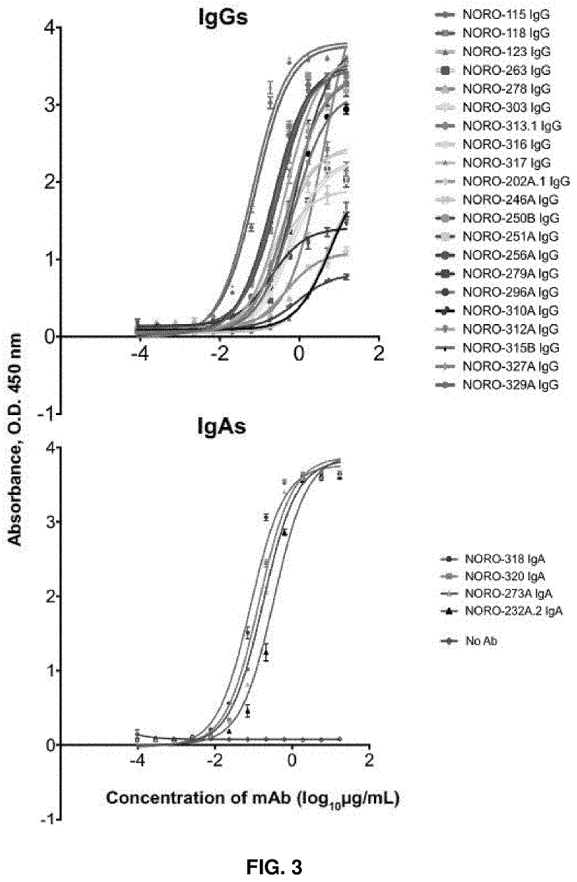

FIG. 4. Characterization of blockade activity of GII.4 Sydney VLPs using isolated mAbs. Blockade potential of serially diluted mAbs or a no-antibody control for GII.4 Sydney 2012 VLP binding to porcine gastric mucin was measured.

FIGS. 5A-B. Half-maximal binding concentrations (EC₅₀) of purified mAbs to GII.4 Sydney 2012 protruding (P) or shell (S) domain. (FIG. 5A) EC₅₀ value indicates the concentration at which half-maximal binding was obtained when tested by ELISA using either recombinantly expressed P domain or shell domain as antigen. mAbs are organized by isotype, IgG or IgA, and arranged in order from lowest to highest EC₅₀ value when binding to GII.4 VLPs. > symbol for IgGs indicates EC₅₀ binding value >150 μg/ml for IgAs. Grey boxes indicate mAbs that exclusively bind to the GII.4 Sydney 2012 shell domain. (FIG. 5B) Binding of mAbs to P or S domain. Graphs are separated by antigen and mAb isotype.

FIG. 6. Competition binding of GII.4 specific mAbs on GST-GII.4 P domain using OctetR HTX System. Epitope binning was performed used biolayer interferometry. GST-tagged GII.4 Sydney 2012 P domain was loaded onto anti-GST tips, then the first antibody was loaded followed by loading of the second antibody. The numerical data has been normalized to a no-antibody buffer control and indicates percent binding of the second antibody in the presence of the first antibody. Yellow, green and magenta boxes indicate potential competition binding groups.

FIG. 7. Predicted blockade epitopes on amino acid sequence alignment of GII.4 Houston 2002 and Sydney 2012. Amino acid sequence alignment of the major capsid protein of GII.4 Houston 2002 (SEQ ID NO: 97) and Sydney 2012 (SEQ ID NO: 98) noroviruses. Boxes indicate predicted blockade epitopes described in (Lindesmith et al., 2012a). Epitope A is amino acids 294, 296-298, 368 and 372. Epitope B is amino acids 333 and 382. Epitope C is amino acids 340 and 376. Epitope D is amino acids 393 and 395. Epitope E is amino acids 407, 412 and 413.

FIGS. 8A-B. Half-maximal binding and blockade concentrations (EC₅₀) of purified mAbs to GII.4 Houston 2002 VLPs. (FIG. 8A) Listed are the EC50 of each mAb for binding or blockade activity using GII.4 Houston VLPs. > symbols indicate blockade EC₅₀ values >100 μg/mL. (FIG. 8B) Serial dilutions of each mAb and a no-antibody control were used to measure blockade activity.

FIGS. 9A-B. Binding activity of cross-reactive human mAbs to GI and GII VLPs. An indirect ELISA was used to assess binding activity of 12 human mAbs to GI.1, GI.2, GI.3, GII.3, GII.4, GII.6, GII.13 or GII.17 VLPs. (FIG. 9A Half-maximal effective concentration (EC₅₀) for binding to VLPs of indicated genotype. Listed are the isotype, light chain and EC₅₀ value to GI.1, GI.2, GI.3, GII.3, GII.4, GII.6, GII.13 or GII.17 VLPs. The > symbol indicates binding was not detected at the highest concentration tested, 500 nm. Greater EC₅₀ values are in the lightest shade of orange and lowest EC₅₀ values are in the darkest shade of orange. (FIG. 9B) Representative ELISA binding curves are shown for indicated genotype.

FIGS. 10A-B. Blockade activity of cross-reactive human mAbs for GI or GII VLPs. Blocking of VLP binding to PGM was used as a surrogate system to test neutralization of GI.3, GII.4, GII.6, or GII.17 VLPs using the indicated human mAbs. (FIG. 10A) Half-maximal effective concentrations (EC₅₀) for cross-reactive mAbs when blocking GI or GII VLPs from binding to PGM. The > symbol indicates the blocking EC₅₀ value was greater than 1,000 nm. (FIG. 10B) Blockade activity was tested using serial dilutions of each mAb.

FIG. 11. Half-maximal effective concentrations (EC₅₀) for binding of 12 cross-reactive human mAbs to protruding or shell domain. GI.3, GII.4, GII.6 or GII.17 NoV strain protruding or shell domain proteins were used as antigen in an indirect ELISA. The > symbol indicates binding was not detected at the highest concentration tested, 500 nm. ND, not determined; P, protruding domain; S, shell domain; P/S, protruding and shell domain.

FIGS. 12A-D. NORO-320 Fab in complex with GII.4 P domain. (FIG. 12A) X-ray crystal structure of NORO-320-GII.4 P domain complex. The two P domain subunits in the dimer are colored in blue and green. NORO-320 Fab (yellow-heavy chain and red-light chain) along with two molecules of H-type 1 pentasaccharide (stick model) modeled to indicate the glycan binding sites for reference. Depicted are side and top views of the complex. (FIG. 12B) Side view of NORO-320 in complex with GII.4 P domain showing the interacting Fab residues (stick model) with the light and heavy chain residues shown in red and yellow, respectively, interacting. (FIG. 12C) Close-up view of the Fab binding site (black box in B) showing the P domain residues (stick models) which interact with the Fab. Residues in the two-fold related subunit (green) is hyphenated. (FIG. 12D) Close up view of the Fab residues (light chain-red, heavy chain yellow) that interact with the GII.4 P domain (shown in surface representation).

FIGS. 13A-C. Molecular interactions between NORO-320 and GII.4 P domain. (FIG. 13A) Antibody plot analysis using the program LigPlot+ v.2.1 (Laskowski et al., 2011). The P2 subdomain residues in the 2-fold related subunit are hyphenated. The hydrogen bonds are shown as green dashed lines, and the hydrophobic contacts are short spokes radiating from each atom or residue. (FIGS. 13B-C) The interactions of P1 or P2 subdomains with NORO-320. The side chains of mAb and P domain are represented with ball-and-stick and stick models, respectively, and colored as in FIG. 4. The hydrogen bonds are shown as black dashed lines.

FIG. 14. Amino acid sequence alignment of the protruding domain of GI.1, GII.3, GII.4, GII.13 and GII.17 strains of human NoV. The protruding domain amino acid sequences of GII.3, GII.4, GII.13 and GII.17, the GII strains tested for which NORO-320 exhibited reactivity, and GI.1 are aligned. Boxed in red are the 18 residues identified on GII.4 that interact with the highly cross-reactive mAb NORO-320. Boxed in gold and blue are the residues previously reported to be involved in GII.4 HBGA binding (Shanker et al., 2011). [GI.1=SEQ ID NO: 57; GII.3=SEQ ID NO: 58; GII.4=SEQ ID NO: 59; GII.6=SEQ ID NO: 60; GII.13=SEQ ID NO: 61; GII.17=SEQ ID NO: 62]

FIG. 15. Blockade of GII.4 VLPs by NORO-320 is a result of steric hindrance. NORO-320 was expressed recombinantly as Fab or IgG forms and purified. GII.4 VLPs were pre-incubated with either NORO-320 Fab, IgG or the original hybridoma-secreted dimeric IgA and added to wells that had been coated previously with PGM. Half-maximal concentrations (EC₅₀) for the three antibodies tested are listed. The > symbol indicates blockade EC₅₀ value was greater than 1,000 nm.

FIG. 16. Verification of molecular assembly of recombinantly expressed NORO-320 variants. NORO-320 was expressed recombinantly and purified in Fab or IgG forms. Fab, IgG and hybridoma dimeric IgA variants were resolved on a SDS-PAGE gel under non-reducing conditions. Recombinant dimeric IgA, IgG and Fab 4C10 was used as a control.

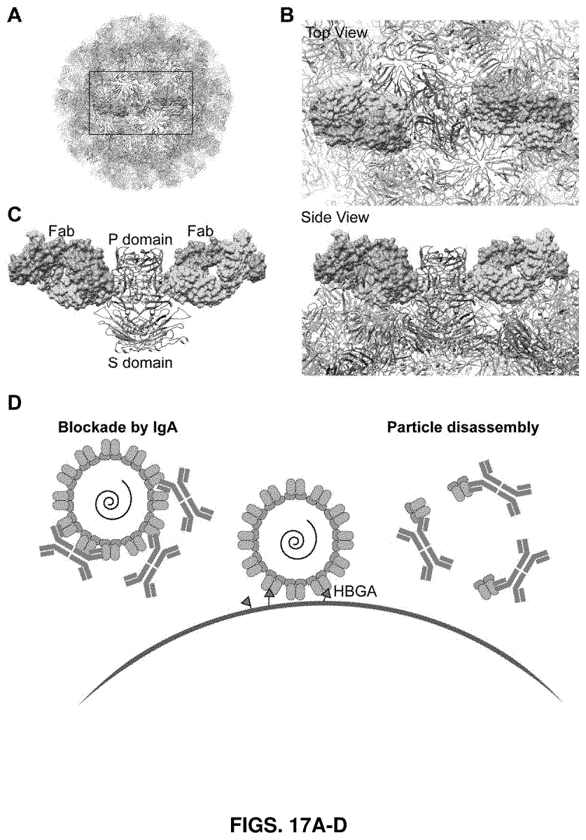

FIGS. 17A-D. Modeling of NORO-320 Fab bound to GII.4 particle. (FIG. 17A) Superimposition of GII.4 P domain/NORO-320 Fab complex onto Norwalk virus capsid (PDB ID: 1IHM). P domain and S domain are colored in cyan and gray, respectively. NORO-320 Fab is shown in surface representation with light chain and heavy chain in orange and yellow, respectively. (FIG. 17B-C) Close-up views of a VP1 dimer (green and blue chains) with two molecules of NORO-320 Fab. (FIG. 17D) A schematic of proposed model for the neutralization of GII strains by NORO-320 IgA.

FIGS. 18A-B. Binding activity of cross-reactive human mAbs to GI and GII VLPs. An indirect ELISA was used to assess the binding activity of 12 human mAbs to GI.1, GI.2, GI.3, GII.3, GII.4, GII.6, GII.13 or GII.17 VLPs. (FIG. 18A) Half-maximal effective concentration (EC₅₀) for binding to VLPs of the indicated genotype. Listed are the isotype, light chain and EC₅₀ value to GI.1, GI.2, GI.3, GII.3, GII.4, GII.6, GII.13 or GII.17 VLPs. The > symbol indicates binding was not detected at the highest concentration tested, 500 nm. Greater EC₅₀ values are in the lightest shade of orange and lowest EC₅₀ values are in the darkest shade of orange. (FIG. 18B) Representative ELISA binding curves are shown for indicated genotype. The binding curve for NORO-320 IgA, which is studied in detail here, is highlighted.

FIGS. 19A-B. Blocking activity of cross-reactive human mAbs for GI or GI′ VLPs. Blocking of VLP binding to PGM was used as a surrogate system to test neutralization of GI.3, GII.4, GII.6, or GII.17 VLPs using the indicated human mAbs. (FIG. 19A) Half-maximal effective concentrations (EC₅₀) for cross-reactive mAbs when blocking GI or GII VLPs from binding to PGM. EC₅₀ values were calculated using a sigmoidal dose-response nonlinear regression analysis after log transformation of the mAb concentrations using GraphPad Prism v 7.0 software. The > symbol indicates the blocking EC₅₀ value was greater than 1,000 nm. FIG. 19B) Blocking activity was tested using serial dilutions of each mAb.

FIG. 20. Half-maximal effective concentrations (EC₅₀) for binding of 12 cross-reactive human mAbs to protruding or shell domain. GI.3, GII.4, GII.6 or GII.17 HuNoV strain protruding or shell domain proteins were used as antigen in an indirect ELISA. The > symbol indicates binding was not detected at the highest concentration tested, 500 nM. ND, not determined; P, protruding domain; S, shell domain; P/S, protruding and shell domain.

FIG. 21. HBGA blocking of GII.4 VLPs by NORO-320 is a result of steric hindrance. NORO-320 was expressed recombinantly as Fab (rFab) or IgG (rIgG) forms and purified. GII.4 VLPs were pre-incubated with either NORO-320 rFab, rIgG or the original hybridoma-secreted (Hyb) dimeric IgA and added to wells that had been coated previously with PGM. Half-maximal concentrations (EC₅₀) for the three antibodies tested are listed. The > symbol indicates blocking EC₅₀ value was greater than 1,000 nm.

FIG. 22. Neutralization of GII.4 and GII.17 in human intestinal enteroid system. HuNoV was mixed with an equal volume of medium or dilutions of the indicated antibody, and then incubated at 37° C. for 1 hr. Human intestinal enteroid monolayers were inoculated with each virus-antibody mixture for 1 hr at 37° C. in the presence of 500 μM GCDCA. The monolayers were washed twice and then cultured in the presence of GCDCA for 24 hr. Compiled data from two experiments are presented. Error bars denote standard deviation and individual points are shown. Percent reduction in viral genome equivalents (GEs) relative to medium (100%) was determined. The dotted line represents 50% neutralization. Significance relative to the control was determined using Student's t-test (***, p<0.001; *, p<0.05; n.s., not significant).

FIGS. 23A-D. NORO-320 Fab in complex with GII.4 P-domain. (FIG. 23A) X-ray crystal structure of the NORO-320-GII.4 P-domain complex. The two P-domain subunits in the dimer are colored in blue and green. NORO-320 Fab (yellow-heavy chain and red-light chain) along with two molecules of H-type 1 pentasaccharide (stick model) are modeled to indicate the glycan binding sites for reference. Depicted are side and top views of the complex. (FIG. 23B) Side view of NORO-320 in complex with GII.4 P-domain showing the interacting Fab residues (stick model) with the light and heavy chain residues shown in red and yellow, respectively, interacting. (FIG. 23C) Close-up view of the Fab binding site (black box in B) showing the P-domain residues (stick models) that interact with the Fab. Residues in the two-fold related subunit (green) is hyphenated. (FIG. 23D) Close-up view of the Fab residues (light chain-red, heavy chain yellow) that interact with the GII.4 P-domain (shown in surface representation).

FIGS. 24A-C. Molecular interactions between NORO-320 and GII.4 P-domain. (FIG. 24A) Antibody plot analysis using the program LigPlot+ v.2.1⁴². The P2 subdomain residues in the 2-fold related subunit are hyphenated. The hydrogen bonds are shown as green dashed lines, and the hydrophobic contacts are short spokes radiating from each atom or residue. (FIGS. 24B-C) The interactions of P1 or P2 subdomains with NORO-320. The side chains of mAb and P-domain are represented with ball-and-stick and stick models, respectively, and colored as in FIG. 4. The hydrogen bonds are shown as black dashed lines.

FIG. 25. Amino acid sequence alignment of the protruding domain of GI.1, GII.3, GII.4, GII.13 and GII.17 strains of HuNoV. The protruding domain amino acid sequences of GII.3, GII.4, GII.13 and GII.17, the GII strains tested for which NORO-320 exhibited reactivity, and GI.1 are aligned. The 18 residues identified on GII.4 that interact with the highly cross-reactive mAb NORO-320 are boxed in red. The residues previously reported to be involved in GII.4 HBGA binding⁴³ are boxed in gold and blue. The recently identified low-affinity bile acid binding site⁴⁴ is boxed in green

FIGS. 26A-B. Dynamic light scattering of mAb NORO-320 and GII.4 Sydney VLP. The hydrodynamic diameters of treated or untreated GII.4 HuNoV VLPs were measured using dynamic light scattering (DLS) on a ZetaSizer Nano instrument (Malvern Instruments, UK). (FIG. 26A) Complete dynamic light scattering profile of the four tested conditions: GII.4 HOV VLP alone or in complex with 22D2 IgG, NORO-320 IgA or Fab at a molar ratio 1:1 or 1:10. A dengue virus-specific antibody, 2D22 IgG, was used as a control. (FIG. 26B) Average diameters were calculated for each sample condition using Zetasizer software. Samples were diluted to a final concentration of 330 nM for each component in phosphate-buffered saline pH 6, and a 3,300 nM concentration of NORO-320 Fab was prepared for the condition labeled VLP:NORO-320 Fab (1:10). Three×12 measurement runs were performed with standard settings (refractive index 1.335, viscosity 0.9, temperature 25° C.) for each time point.

FIG. 27. Verification of molecular assembly of recombinantly expressed NORO-320 variants. NORO-320 was expressed recombinantly and purified in Fab or IgG forms. Fab, IgG and hybridoma dimeric IgA variants were resolved on an SDS-PAGE gel under non-reducing conditions. Recombinant dimeric IgA, IgG and Fab 4C10 was used as a control.

FIGS. 28A-D. Modeling of NORO-320 Fab bound to GII.4 particle. (FIG. 28A) Superimposition of GII.4 P-domain/NORO-320 Fab complex onto Norwalk virus capsid (PDB ID: 1IHM). P or S-domains are colored in cyan or gray, respectively. NORO-320 Fab is shown in surface representation with light or heavy chains in orange or yellow, respectively. (FIGS. 28B-C) Close-up views of a VP1 dimer (green and blue chains) with two molecules of NORO-320 Fab. (FIG. 28D) A schematic of proposed model for the neutralization of GII strains by NORO-320 IgA or Fab.

FIGS. 29A-F. Changes in bis-ANS binding upon mixing GII.4 VLP with antibody. Purified VLP (30 ug/ml-1, 0.5 μM concentration of the VP1) or 0.5 μM purified antibody (NORO-320 Fab, IgA, or 22D2 control) diluted in PBS buffer pH 6.0 was incubated at 25° C. (FIG. 29A, FIG. 29C, FIG. 29E) or 37° C. (FIG. 29B, FIG. 29D, FIG. 29F) for 10 minutes to allow for temperature equilibration. At time zero, bis-ANS was added to the indicated VLP, antibody, or VLP preincubated with antibody. Fluorescence was recorded every 30 seconds continuously for 15 minutes at excitation and emission wavelengths of 395 and 495 nm, respectively. Stabilized fluorescence intensities measured during the last minute for each sample were averaged and presented as a bar graph. The means +−SE (n=3) are shown.

FIG. 30. Dynamic light scattering of mAb NORO-320 and GII.4 VLP with temperature and pH variation. Samples were diluted to a final concentration of 330 nM for each component in phosphate-buffered saline pH 6, pH 7, or pH 8 and incubated at the designated temperature for 30 minutes. Three×12 measurement runs were performed with standard settings (refractive index 1.335, viscosity 0.9, temperature 25° C., 37° C., or 40° C.). The hydrodynamic diameters of treated or untreated GII.4 HuNoV VLPs were measured using dynamic light scattering (DLS) on a ZetaSizer Nano instrument (Malvern Instruments, UK). Z-Average hydrodynamic diameters were calculated for each sample condition using Zetasizer software.

TABLE A
Isotype, light chain and ELISA binding characterization of
GII.4 P VLP-specific human mAbs.

| | mAb clone, | Light | EC₅₀ |
|---|---|---|---|
| Isotype | NORO- | chain | (μg/mL) |
| IgG | 115 | K | 0.1 |
| | 313.1 | K | 0.1 |
| | 246A | λ | 0.2 |
| | 250B | λ | 0.2 |
| | 279A | λ | 0.2 |
| | 329A | λ | 0.2 |
| | 118 | λ | 0.3 |
| | 316 | λ | 0.3 |
| | 202A.1 | λ | 0.4 |
| | 312A | λ | 0.4 |
| | 317 | λ | 0.4 |
| | 303 | λ | 0.5 |
| | 263 | λ | 0.6 |
| | 296A | λ | 0.6 |
| | 327A | λ | 0.6 |
| | 315B | λ | 0.7 |
| | 251A | λ | 0.9 |
| | 256A | λ | 0.9 |
| | 278 | λ | 4.0 |
| | 123 | λ | 5.4 |
| | 310A | K | 6.2 |
| IgA | 318 | K | 0.1 |
| | 320 | K | 0.1 |
| | 273A | K | 0.2 |
| | 232A.2 | K | 0.3 |

Isotype, light chain and ELISA binding characterization of GII.4 P VLP-specific human mAbs. Listed are the half-maximal concentrations (EC50) at which half-maximal binding was obtained when tested by ELISA using VLPs as the antigen. MAbs are organized by isotype (IgG or IgA) and arranged in order from lowest to highest EC50 value when binding to GII.4 VLPs.

TABLE X
Antibody variable gene usage for GII.4 Sydney 2012 VLP-binding mAbs.

| | | | Heavy Chain | | | | | | |
|---|---|---|---|---|---|---|---|---|---|
| | NORO | Isotype/ | | V gene | | J gene | | | |
| | mAb | Light | | % | | % | | | HCDR |
| Donor | clone | chain | V gene | identity | J gene | identity | D gene | Junction (SEQ ID NO:) | length |
| HD329 | 115 | IgG/K | V3-11*01 F | 89.2 | J5*02 F | 94.1 | D6-13*01 F | CARDRLPASGSHWFHPW (63) | 8.8.15 |
| HD331 | 313.1 | IgG/K | V3-11*06 F | 92.7 | J6*02 F | 80.7 | D1-1*01 F | CARMGRLELERRPHYYYPLDVW | 8.8.20 |
| | | | | | | | | (64) | |
| HD334 | 250B | IgG/λ | V3-11*06 F | 95.5 | J4*02 F | 97.9 | D1-26*01 F | CARATSQGATSYYFDSW (65) | 8.8.15 |
| HD337 | 279A | IgG/λ | V3-30*03 F | 91.7 | J6*02 F | 79.0 | D2-8*01 F | CAKVEIHYYSNSLLGMDVW | 8.8.17 |
| | | | | | | | | (66) | |
| HD331 | 329A | IgG/λ | V3-11*06 F | 96.2 | J4*02 F | 91.7 | D3-10*01 F | CARYNYYGSGSFVFDYW (67) | 8.8.15 |
| 990 | 202A.1 | IgG/λ | V1-2*02 F | 93.3 | J3*01 F | 92.0 | D7-27*01 F | CARDLLRNWGDHDAFDVW | 8.7.16 |
| | | | | | | | | (68) | |
| HD331 | 312A | IgG/λ | V3-11*06 F | 94.1 | J4*02 F | 89.6 | D2-15*01 F | CARDAQYCSGGRCYLVFDYW | 8.8.18 |
| | | | | | | | | (69) | |
| HD331 | 317 | IgG/λ | V4-34*01 F | 91.2 | J5*02 F | 90.0 | D3-16*02 F | CARGQMRTRGALFRRFDPW | 8.7.17 |
| | | | | | | | | (70) | |
| HD333 | 303 | IgG/λ | V3-30*01 F | 97.6 | J6*02 F | 80.7 | D3-10*01 F | CARDCRVGWVFTYGMDVW | 8.8.16 |
| | | | | | | | | (71) | |
| HD333 | 296A | IgG/λ | V3-11*05 F | 95.1 | J1*01 F | 86.3 | D3-10*01 F | CARYGAEYGSRSFYFLDW | 8.8.16 |
| | | | | | | | | (72) | |
| HD331 | 315B | IgG/λ | V3-11*06 F | 90.3 | J4*02 F | 89.6 | D2-15*01 F | CAREDCHGTSCYSGDW (73) | 8.8.14 |
| HD334 | 251A | IgG/λ | V3-30*03 F | 97.2 | J4*02 F | 91.7 | D6-19*01 F | CAKVRLTSYSIGWFSFDYW | 8.8.17 |
| | | | | | | | | (74) | |
| HD334 | 256A | IgG/λ | V3-30*03 F | 96.2 | J3*02 F | 82.0 | D5-18*01 F | CAKDFLRVYSYGWHSFDIW | 8.8.17 |
| | | | | | | | | (75) | |
| HD337 | 278 | IgG/λ | V3-30*03 F | 100 | J4*02 F | 93.8 | D6-13*01 F | CAKVTIIAAADLLDYW (76) | 8.8.14 |
| HD335 | 123 | IgG/λ | V3-30*04 F | 94.1 | J6*02 F | 82.6 | D2-15*01 F | CARVTGDCTGNRCSYWAYYYYG | 8.8.24 |
| | | | | | | | | LDVW (77) | |
| HD331 | 310A | IgG/K | V3-66*01 F | 94.4 | J4*02 F | 93.8 | D3-22*01 F | CTRDPSQYYDSRGHYYQTFTPS | 8.7.24 |
| | | | | | | | | FDSW (78) | |
| HD331 | 320 | IgA/K | V1-69*01 F | 95.5 | J6*02 F | 77.4 | D3-10*01 F | CARDRVPSYSPSRRFSTKGAMW | 8.8.28 |
| | | | | | | | | GKYGMDVW (79) | |

| | Light Chain | | | | | |
|---|---|---|---|---|---|---|
| | | | | J Gene | Junction | LCDR |
| Donor | V gene | V gene length | J gene | % identity | (SEQ ID NO:) | length |
| HD329 | V4-1*01 F | 95.8 | J4*01 F | 97.2 | CQQYYNSPLAF (80) | 12.3.9 |
| HD331 | V2-28*01 F | 97.6 | J2*02 F | 97.2 | CMQALQTRTF (81) | 11.3.8 |
| HD334 | V1-40*01 F | 95.8 | J2*01 F | 89.5 | CQSYDRSVSGSAVF (82) | 9.3.12 |
| HD337 | V1-47*01 F | 92.6 | J3*02 F | 94.6 | CATLDINMTWVF (83) | 8.3.10 |
| HD331 | V1-44*01 F | 96.5 | J3*02 F | 100 | CAAWDDSLNGWVF (84) | 8.3.11 |
| 990 | V1-47*01 F | 97.4 | J2*01 F | 100 | CSAWDDSLSGPVF (85) | 1.3.11 |
| HD331 | V1-40*01 F | 96.2 | J2*01 F | 97.1 | CQSYDNRLRVF (86) | 9.3.9 |
| HD331 | V3-25*03 F | 93.6 | J3*02 F | 89.5 | CQSVDTRGTYKVF (87) | 6.3.11 |
| HD333 | V2-8*01 F | 98.3 | J2*01 F | 100 | CSSYAGTYNCVVF (88) | 9.3.11 |
| HD333 | V1-40*01 F | 96.9 | J2*01 F | 92.1 | CQSYDSRLSSNVVF (89) | 9.3.12 |
| HD331 | V1-40*01 F | 97.2 | J3*02 F | 92.1 | CQSYDRSLSKSRVF (90) | 9.3.12 |
| HD334 | V1-51*01 F | 97.9 | J3*02 F | 91.9 | CGTWDTSLRACLF (91) | 8.3.11 |
| HD334 | V1-51*01 F | 95.8 | J3*02 F | 91.1 | CGTWDLSLTAGWVF (92) | 8.3.12 |
| HD337 | V1-40*01 F | 100 | J2*01 F | 97.4 | CQSYDSSLSGPVVF (93) | 9.3.12 |
| HD335 | V2-8*01 F | 97.6 | J1*01 F | 94.7 | CGSYAGSTTSGYVF (94) | 9.3.12 |
| HD331 | V1-17*03 F | 97.5 | J4*01 F | 100 | CLQHDTYPLTF (95) | 6.3.9 |
| HD331 | V2-28*01 F | 99.3 | J1*01 F | 100 | CMQALQTPRTF (96) | 11.3.9 |

Heavy and light chain variable gene regions were sequenced. All of the mAbs had unique heavy and light complementarity-determining region 3 (CDR3) sequences.

SUPPLEMENTAL TABLE 1
Antibody sequence analysis for 13 cross-reactive human mAbs. Heavy and variable
gene regions were sequenced. All of the mAbs had unique heavy and light chain complementary-
determining region 3 (CDR3) sequences.

| | | Heavy Chain | | | | | | |
|---|---|---|---|---|---|---|---|---|
| NORO | | | V gene | | J gene | | | |
| mAb | | | % | | % | | | HCDR |
| clone | Isotype | V gene | identity | J gene | identity | D gene | JUNCTION | length |
| 155.5 | Ig [symbol] | V4-34*01 F | 100 | J8*02 F | 98.8 | D3-12*01 F | [symbol] | 8.7.22 |
| 166.3 | Ig [symbol] | V4-34*01 F | 100 | J6*02 F | 89.1 | D3-16*01 F | CARGLVDVW | [symbol] |
| 181.2 | Ig [symbol] | V3-74*01 F | 100 | J3*02 F | 92.0 | [symbol] | [symbol] | 8.8.16 |
| 188.2 | Ig [symbol] | V3-30*01 F | 100 | J6*02 F | 90.3 | [symbol] | CARSVIGYYYGMDVW | 8.8.14 |
| 178.5 | Ig [symbol] | [symbol] | 100 | J1*01 F | 90.4 | D4/23*01 ORF | [symbol] | 8.8.13 |
| 187.3 | IgG | V5-51*01 F | 100 | J4*02 F | 87.5 | [symbol] F | [symbol] | 8.8.14 |
| 178.6 | IgG | V3-30*03 F | 100 | J4*02 F | 85.2 | [symbol] F | [symbol] | 8.8.15 |
| [symbol] | IgG | V1-2*02 F | 93.3 | J3*01 F | 92.0 | [symbol] F | [symbol] | 8.7.16 |
| [symbol] | IgG | V3-30*03 F | [symbol] 1.7 | J8*02 F | 79.0 | [symbol] F | [symbol] | 8.8.17 |
| [symbol] | IgG | V3-66*01 F | 94.4 | J4*02 F | 93.8 | [symbol] F | [symbol] | 8.7.24 |
| [symbol] | IgG | V3-30*03 F | 95.8 | J4*02 F | 93.8 | [symbol] ORF | [symbol] | 8.8.10 |
| [symbol] | IgA | [symbol] -34*02 F | 93.6 | J6*02 F | 79.0 | [symbol] F | [symbol] | 8.7.24 |
| [symbol] | IgA | V1-89*01 F | 95.6 | J6*02 F | 77.4 | [symbol] F | [symbol] | 8.8.28 |

| | Light Chain | | | | | |
|---|---|---|---|---|---|---|
| NORO | | V gene | | J gene | | |
| mAb | | % | | % | | LCDR |
| clone | V gene | identity | J gene | identity | AA JUNCTION | length |
| 155.5 | [symbol] F | 100 | [symbol] F | 97.3 | [symbol] | 8.3.9 |
| 166.3 | [symbol] F | 100 | 32*02 F | 100 | [symbol] | 6.3.8 |
| 181.2 | [symbol] F | 100 | 32*01 F | 100 | [symbol] | 8.3.5 |
| 188.2 | [symbol] F | 100 | 32*01 F | 89.6 | [symbol] | 7.8.6 |
| 178.5 | [symbol] F | 100 | 35*01 F | 100 | [symbol] | 6.3.5 |
| 187.3 | [symbol] F | 100 | 33*01 F | 97.4 | [symbol] | 6.3.5 |
| 178.6 | [symbol] F | 96. [symbol] | 32*01 F | 81.6 | [symbol] | 6.3.10 |
| [symbol] | [symbol] F | 97.5 | 32*01 F | 100 | [symbol] | 1.3.11 |
| [symbol] | [symbol] F | 92.8 | 33*02 F | 84.6 | [symbol] | 8.3.10 |
| [symbol] | [symbol] F | 98.0 | 34*02 F | 100 | [symbol] | 6.3.5 |
| [symbol] | [symbol] F | 97.2 | 32*01 F | 94.6 | [symbol] | 9.3.10 |
| [symbol] | [symbol] F | 91.8 | 32*01 F | 99.5 | [symbol] | 7.3.8 |
| [symbol] | [symbol] F | 95.3 | 31*01 F | 100 | [symbol] | 11.3.9 |

(SEQ ID NOS: 31-56) [symbol]
indicates data missing or illegible when filed

TABLE B
Data processing and refinement statistics for GII.4 P
domain-Fab 320 complex.

| Data Collection | |
|---|---|
| Beamline | ALS Beamline 5.0.1 |
| Wavelength, Å | 0.97741 |
| Space group | P21 21 2 |
| Cell dimensions, Å | 119.25, 186.27, 73.44 |
| α, β, γ, ° | 90, 90, 90 |
| Resolution, Å | 50-2.25 (2.29-2.25)^{a} |
| Total Reflections | 1716311 |
| Unique Reflections | 78053 (3854)^{a} |
| Redundancy | 6.5 (6.2)^{a} |
| Completeness (%) | 99.82 |
| <I/sigma> | 15.6875 (2.375)^{a} |
| Rₘₑₐₛ ^{b} | 0.129 (0.846)^{a} |
| Rₚᵢₘ ^{b} | 0.050 (0.340)^{a} |
| Refinement Statistics | |
| Resolution, Å | 50-2.25 (2.29-2.25)^{a} |
| Reflections (work) | 73965 |
| Reflections (Test) | 3926 |
| R_{work} ^{C}/R_{free} ^{d} (%) | 18.08/22.55 |
| No. of Atoms | |
| Protein | |
| P-domain Dimer | 4798 |
| Noro-320 Fab | 6674 |
| Water | 1059 |
| Average B Value (Å²) | |
| P-domain Dimer | 34.2505 |
| Noro-320 Fab | 31.67 |
| Water | 36.085 |
| RMSD from Ideal Geometry | |
| Bond length (Å) | 0.003 |
| Bond angle (°) | 0.614 |
| Ramachandran Statistics^{e} | |
| Favored | 98.38% |
| Outliers | 0.20% |

^{a}Numbers in parentheses refer to the highest resolution shell
^{b} Rₘₑₐₛ = Σhkl {N(hkl)/[N(hkl) − 1]}^{1/2} X Σᵢ|Iᵢ(hkl) − {I(hkl)}|/Σₕₖₗ ΣᵢIᵢ(hkl) and Rₚᵢₘ = Σₕₖₗ (1/(n − 1))^{1/2} Σᵢ|I_{hkl,i} − |/Σₕₖₗ Σi I_{hkl,i}, where Iₕₖₗ,i is the scaled intensity of the i^{th} measurement of reflection h, k, l, is the average intensity for that reflection, and n is the redundancy.
^{C} R_{work} = Σₕₖₗ|Fo − Fc|/Σₕₖₗ|Fo|× 100, where Fo and Fc are the observed and calculated structure factors, respectively.
^{d} R_{free} was calculated as for R_{work}, but on a test set comprising 5% of the data excluded from refinement.
^{e} Calculated with MolProbity (Chen et al., 2010).

### DESCRIPTION OF ILLUSTRATIVE EMBODIMENTS

As discussed above, development of norovirus vaccines has proven challenging. In this study, the inventor describes the isolation and characterization of a panel of human monoclonal antibodies (mAbs) that bind to GII.4 Sydney 2012 VLPs. The majority of these antibodies also block receptor binding, as inferred by their ability to inhibit hemagglutination of human O+ red blood cells (RBCs) or the interaction between GII.4 Sydney 2012 VLPs and porcine gastric mucin (PGM). Both of these assays are surrogate systems for testing HuNoV neutralization (Czako et al., 2012; Reeck et al., 2010). For over 40 years there have been numerous documented attempts to cultivate HuNoVs in vitro, but previously none of them resulted in the establishment of a robust reproducible system of viral growth (Jones et al., 2014; Duizer et al., 2004; Herbst-Kralovetz et al., 2013). Recent breakthroughs in the development of an in vitro replication system using human intestinal organoid technology have now made it possible to cultivate HuNoV and to test inhibition of growth, or neutralization, using antibodies (Ettayebi et al., 2016; Constantini et al., 2018).

Here, the inventor used a human jejunal monolayer culture system to identify antibodies that neutralize live GII.4 Sydney 2012 HuNoV. He identified the first neutralizing human mAbs against norovirus, as well as a panel of human anti-GII.4 Sydney 2012 VLP binding IgGs and the first anti-GII.4 human IgA molecules. Almost 70% of the mAbs that the inventor isolated exhibited a high level of potency, inhibiting GII.4 Sydney 2012 VLPs from binding to PGM at half-maximal effective concentrations (EC₅₀) below 24 μg/mL. He also used this panel of mAbs to identify major antigenic sites on the GII.4 Sydney 2012 major capsid protein.

The broadly cross-reactive naturally-occurring human monoclonal included IgMs, IgAs and IgGs reactive with NoV genogroup I or II (GI or GII). Among the panel the inventor noticed three different binding patterns and identified monoclonal antibodies (mAbs) that neutralized at least one GI or GII NoV strain when using a receptor blocking assay. X-ray crystallography studies of a GII-specific neutralizing mAb revealed the antibody neutralizes not by directly inhibiting receptor binding, but instead through steric hindrance. These data will be useful when designing and evaluating new vaccine candidates. Some of the human mAbs described here also could be used as biologics for the prevention or treatment of chronic NoV infections or severe NoV disease during outbreaks.

These studies contribute new insights into natural human humoral immunity to HuNoVs and provide mAbs that have the potential to be used for diagnostic and therapeutic purposes. These and other aspects of the disclosure are described in detail below.

### I. Norovirus Virus

Norovirus is the most common cause of gastroenteritis. Infection is characterized by diarrhea, vomiting, and stomach pain. Blood is not usually present. Fever or headaches may also occur. This usually develops 12 to 48 hours after being exposed. Recovery typically occurs within 1 to 3 days. Complications may include dehydration.

The virus is usually spread by the fecal-oral route. This may be by contaminated food or water or person-to-person contact. It may also spread via contaminated surfaces or through the air. Risk factors include unsanitary food preparation. Diagnosis is generally based on symptoms. Confirmatory testing may be done for public health purposes.

Prevention involves proper hand washing and disinfection of contaminated surfaces. Alcohol-based hand sanitizers are less effective. A vaccine does not exist. There is no specific treatment. Efforts involve supportive care such as drinking sufficient fluids or intravenous fluids. Oral rehydration solutions are the preferred fluids to drink.

Norovirus results in about 685 million cases of disease and 200,000 deaths globally a year. It is common both in the developed and developing world. Those under the age of five are most often affected and in this group it results in about 50,000 deaths in the developing world. Disease more commonly occurs in winter months. It often occurs in outbreaks, especially among those living in close quarters. In the United States it is the cause of about half of food-borne disease outbreaks. The disease is named after Norwalk, Ohio, where an outbreak occurred in 1968.

Indeed, Norovirus causes about 18% of all cases of acute gastroenteritis worldwide. It is relatively common in developed countries and in low-mortality developing countries (20% and 19% respectively) compared to high-mortality developing countries (14%). Proportionately it causes more illness in people in the community or in hospital outpatients (24% and 20% respectively) as compared with hospital inpatients (17%) in whom other causes are more common.

Norovirus is a common cause of epidemics of gastroenteritis on cruise ships. The US Centers for Disease Control and Prevention through its Vessel Sanitation Program record and investigate outbreaks of gastrointestinal illness—mostly caused by norovirus—on cruise ships with both a U.S. and foreign itinerary; there were 12 in 2015, and 10 from 1 January to 9 May 2016. An outbreak may affect over 25% of passengers, and a smaller proportion of crew members.

Norovirus infection is characterized by nausea, vomiting, watery diarrhea, abdominal pain, and in some cases, loss of taste. A person usually develops symptoms of gastroenteritis 12 to 48 hours after being exposed to norovirus. General lethargy, weakness, muscle aches, headaches, and low-grade fevers may occur. The disease is usually self-limiting, and severe illness is rare. Although having norovirus can be unpleasant, it is not usually dangerous and most who contract it make a full recovery within two to three days.

A. Transmission

Noroviruses are transmitted directly from person to person (62-84% of all reported outbreaks) and indirectly via contaminated water and food. They are extremely contagious, and fewer than twenty virus particles can cause an infection (some research suggests as few as five). Transmission can be aerosolized when those stricken with the illness vomit, and can be aerosolized by a toilet flush when vomit or diarrhea is present; infection can follow eating food or breathing air near an episode of vomiting, even if cleaned up. The viruses continue to be shed after symptoms have subsided and shedding can still be detected many weeks after infection.

Vomiting, in particular, transmits infection effectively, and appears to allow airborne transmission. In one incident, a person who vomited spread infection across a restaurant, suggesting that many unexplained cases of food poisoning may have their source in vomit. In December 1998, 126 people were dining at six tables; one woman vomited onto the floor. Staff quickly cleaned up, and people continued eating. Three days later others started falling ill; 52 people reported a range of symptoms, from fever and nausea to vomiting and diarrhea. The cause was not immediately identified. Researchers plotted the seating arrangement: more than 90% of the people at the same table as the sick woman later reported becoming ill. There was a direct correlation between the risk of infection of people at other tables and how close they were to the sick woman. More than 70% of the diners at an adjacent table fell ill; at a table on the other side of the restaurant, the attack rate was still 25%. The outbreak was attributed to a Norwalk-like virus (norovirus). Other cases of transmission by vomit were later identified.

In one outbreak at an international scout jamboree in the Netherlands, each person with gastroenteritis infected an average of 14 people before increased hygiene measures were put in place. Even after these new measures were enacted, an ill person still infected an average of 2.1 other people. A US CDC study of 11 outbreaks in New York State lists the suspected mode of transmission as person-to-person in seven outbreaks, foodborne in two, waterborne in one, and one unknown. The source of waterborne outbreaks may include water from municipal supplies, wells, recreational lakes, swimming pools and ice machines.

Shellfish and salad ingredients are the foods most often implicated in norovirus outbreaks. Ingestion of shellfish that have not been sufficiently heated—under 75° C. (167° F.)—poses a high risk for norovirus infection. Foods other than shellfish may be contaminated by infected food handlers. Many norovirus outbreaks have been traced to food that was handled by one infected person.

B. Classification

Noroviruses (NoV) are a genetically diverse group of single-stranded positive-sense RNA, non-enveloped viruses belonging to the family Caliciviridae. According to the International Committee on Taxonomy of Viruses, the genus Norovirus has one species, which is called Norwalk virus. Serotypes, strains and isolates include Norwalk virus, Hawaii virus, Snow Mountain virus, Mexico virus, Desert Shield virus, Southampton virus, Lordsdale virus and Wilkinson virus.

Noroviruses commonly isolated in cases of acute gastroenteritis belong to two genogroups: genogroup I (GI) includes Norwalk virus, Desert Shield virus and Southampton virus; and II (GII), which includes Bristol virus, Lordsdale virus, Toronto virus, Mexico virus, Hawaii virus and Snow Mountain virus.

Noroviruses can genetically be classified into seven different genogroups (GI, GII, GIII, GIV, GV, GVI, and GVII), which can be further divided into different genetic clusters or genotypes. For example, genogroup II, the most prevalent human genogroup, presently contains 19 genotypes. Genogroups I, II and IV infect humans, whereas genogroup III infects bovine species, and genogroup V has recently been isolated in mice.

Most noroviruses that infect humans belong to genogroups GI and GII. Noroviruses from Genogroup II, genotype 4 (abbreviated as GII.4) account for the majority of adult outbreaks of gastroenteritis and often sweep across the globe. Recent examples include US95/96-US strain, associated with global outbreaks in the mid- to late-1990s; Farmington Hills virus associated with outbreaks in Europe and the United States in 2002 and in 2004; and Hunter virus which was associated with outbreaks in Europe, Japan and Australasia. In 2006, there was another large increase in NoV infection around the globe. GII.17 emerged and became predominant in many areas in Asia and was detected in other countries in 2014-2016. Since then a GII.2 strains has emerged in a number of countries. Reports have shown a link between the expression of human histo-blood group antigens (HBGAs) and the susceptibility to norovirus infection. Studies have suggested the viral capsid of noroviruses may have evolved from selective pressure of human HBGAs.

One study suggests the protein MDA-5 may be the primary immune sensor that detects the presence of noroviruses in the body. Some people have common variations of the MDA-5 gene that could make them more susceptible to norovirus infection. Another study suggested a specific genetic version of norovirus (which would not be distinguishable from other types of the virus using standard viral antibody tests) interacts with a specific mutation in the ATG16L1 gene to help trigger symptomatic Crohn's disease in mice that have been subjected to a chemical that causes intestinal injury similar to the process in humans. (There are other similar ways for such diseases to happen like this, and this study in itself does not prove norovirus causes Crohn's disease in humans).

C. Structure

Viruses in Norovirus are non-enveloped, with icosahedral geometries. Capsid diameters vary widely, from 23-40 nm in diameter. The larger capsids (38-40 nm) exhibit T=3 symmetry and are composed of 180 VP1 proteins. Small capsids (23 nm) show T=1 symmetry and are composed of 60 VP1 proteins. The virus particles demonstrate an amorphous surface structure when visualized using electron microscopy.

Noroviruses contain a linear, non-segmented, positive-sense RNA genome of approximately 7.5 kbp, encoding a large polyprotein which is cleaved into six smaller nonstructural proteins (NS1/2 to NS7) by the viral 3C-like protease (NS6), a major structural protein (VP1) of about 58-60 kDa and a minor capsid protein (VP2).

The most variable region of the viral capsid is the P2 domain, which contains antigen-presenting sites and carbohydrate-receptor binding regions. The estimated mutation rate (1.21×10⁻² to 1.41×10⁻² substitutions per site per year) in this virus is high even compared with other RNA viruses. In addition, a recombination hotspot exists at the ORF1-ORF2 (VP1) junction.

D. Lifecycle and Persistence

Viral replication is cytoplasmic. Entry into the host cell is achieved by attachment to host receptors, which mediates endocytosis. Replication follows the positive stranded RNA virus replication model. Positive stranded RNA virus transcription is the method of replication. Translation takes place by leaky scanning and RNA termination-reinitiation. Humans and other mammals serve as the natural host. Transmission routes are fecal-oral and contamination.

The norovirus can survive for long periods outside a human host depending on the surface and temperature conditions: it can stay for weeks on hard surfaces, and up to twelve days on contaminated fabrics, and it can survive for months, maybe even years in contaminated still water. A 2006 study found the virus remained on surfaces used for food preparation seven days after contamination.

E. Pathophysiology

When a person becomes infected with norovirus, the virus is replicated within the small intestine. After approximately one to two days, norovirus infection symptoms can appear. The principal symptom is acute gastroenteritis that develops between 12 and 48 hours after exposure, and lasts for 24-72 hours. The disease is usually self-limiting, and characterized by nausea, forceful vomiting, watery diarrhea, and abdominal pain, and in some cases, loss of taste. General lethargy, weakness, muscle aches, headache, coughs, and low-grade fever may occur.

Severe illness is rare; although people are frequently treated at the emergency ward, they are rarely admitted to the hospital. The number of deaths from norovirus in the United States is estimated to be around 300 each year, with most of these occurring in the very young, the elderly, and persons with weakened immune systems. Symptoms may become life-threatening in these groups if dehydration or electrolyte imbalance is ignored or not treated.

F. Diagnosis and Detection

Specific diagnosis of norovirus is routinely made by polymerase chain reaction (PCR) assays or quantitative PCR assays, which give results within a few hours. These assays are very sensitive and can detect as few as 10 virus particles. Tests such as ELISA that use antibodies against a mixture of norovirus strains are available commercially, but lack specificity and sensitivity. Due to a lack of specific therapy, the need for expensive stool diagnostics is being questioned by experts if gastroenteritis by noroviruses has already been detected in the environment.

Routine protocols to detect norovirus in clams and oysters by reverse transcription polymerase chain reaction are being employed by governmental laboratories such as the Food and Drug Administration (FDA) in the USA.

G. Prevention

After infection, immunity to the same strain of the virus—the genotype—protects against reinfection for between 6 months to 2 years. This immunity does not fully protect against infection with the other diverse genotypes of the virus.

Hand washing with soap and water is an effective method for reducing the transmission of norovirus pathogens. Alcohol rubs (>62% ethanol) may be used as an adjunct, but are less effective than hand-washing, as norovirus lacks a lipid viral envelope. Surfaces where norovirus particles may be present can be sanitized with a solution of 1.5% to 7.5% of household bleach in water, or other disinfectants effective against norovirus.

In health-care environments, the prevention of nosocomial infections involves routine and terminal cleaning. Non-flammable alcohol vapor in CO₂ systems is used in health care environments where medical electronics would be adversely affected by aerosolized chlorine or other caustic compounds.

In 2011, the Centers for Disease Control and Prevention (CDC) published a clinical practice guideline addressing strategies for the prevention and control of norovirus gastroenteritis outbreaks in health-care settings. Based on a systematic review of published scientific studies, the guideline presents 51 specific evidence-based recommendations, which were organized into 12 categories: 1) patient cohorting and isolation precautions, 2) hand hygiene, 3) patient transfer and ward closure, 4) food handlers in healthcare, 5) diagnostics, 6) personal protective equipment, 7) environmental cleaning, 8) staff leave and policy, 9) visitors, 10) education, 11) active case-finding, and 12) communication and notification. The guideline also identifies eight high-priority recommendations, and suggests several areas in need of future research.

Ligocyte announced in 2007 that it was working on a vaccine and had started phase 1 trials. The company has since been acquired by Takeda Vaccines. As of 2011, a monovalent nasal vaccine had completed phase I/II trials, while bivalent intramuscular and nasal vaccines were at earlier stages of development. The two vaccines rely on using a virus-like particle that is made of the norovirus capsid proteins in order to mimic the external structure of the virus. Since there is no RNA in this particle, it is incapable of reproducing and cannot cause an infection.

H. Treatment

There is no specific medicine to treat people with norovirus illness. Norovirus infection cannot be treated with antibiotics because it is not a bacterial infection. Treatments aim to avoid complications by measures such as the management of dehydration caused by fluid loss in vomiting and diarrhea, and to mitigate symptoms using antiemetics and antidiarrheals.

### II. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

It will be understood that monoclonal antibodies binding to norovirus will have several applications. These include the production of diagnostic kits for use in detecting and diagnosing norovirus infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce norovirus-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and Chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A. When the antibody neutralizes norovirus, antibody escape mutant variant organisms can be isolated by propagating norovirus in vitro or in animal models in the presence of high concentrations of the antibody. Sequence analysis of the norovirus gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially, overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-norovirus antibody, the above-described binding methodology is performed in two orientations: In a first orientation, the reference antibody is allowed to bind to the norovirus antigen under saturating conditions followed by assessment of binding of the test antibody to the norovirus antigen. In a second orientation, the test antibody is allowed to bind to the norovirus antigen molecule under saturating conditions followed by assessment of binding of the reference antibody to the norovirus molecule. If, in both orientations, only the first (saturating) antibody is capable of binding to the norovirus, then it is concluded that the test antibody and the reference antibody compete for binding to the norovirus. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740; Davies J. et al. (2001) “Expression Of GnTIII In A Recombinant Anti- CD20 CHO Production Cell Line: Expression Of Antibodies With Altered Glycoforms Leads To An Increase In ADCC Through Higher Affinity For FC Gamma RIII,” Biotechnology & Bioengineering 74(4): 288-294). Methods of altering carbohydrate contents are known to those skilled in the art, see, e.g., Wallick, S. C. et al. (1988) “Glycosylation Of A VH Residue Of A Monoclonal Antibody Against Alpha (1-6) Dextran Increases Its Affinity For Antigen,” J. Exp. Med. 168(3): 1099-1109; Tao, M. H. et al. (1989) “Studies Of Aglycosylated Chimeric Mouse- Human IgG. Role Of Carbohydrate In The Structure And Effector Functions Mediated By The Human IgG Constant Region,” J. Immunol. 143(8): 2595-2601; Routledge, E. G. et al. (1995) “The Effect Of Aglycosylation On The Immunogenicity Of A Humanized Therapeutic CD3 Monoclonal Antibody,” Transplantation 60(8):847-53; Elliott, S. et al. (2003) “Enhancement Of Therapeutic Protein In Vivo Activities Through Glycoengineering,” Nature Biotechnol. 21:414-21; Shields, R. L. et al. (2002) “Lack Of Fucose On Human IgG N-Linked Oligosaccharide Improves Binding To Human Fcgamma RIII And Antibody-Dependent Cellular Toxicity,” J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2a phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)2) that are produced, for example, by the proteolytic cleavage of the mAbs, or single-chain immunoglobulins producible, for example, via recombinant means. F(ab′) antibody derivatives are monovalent, while F(ab′)2 antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection; however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′).sub.2 bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998), doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1).sub.n-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains V_{L} and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
   The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3 ξ-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third-generation” CARs combine multiple signaling domains, such as CD3z-CD28-41BB or CD3z-CD28-OX40, to further augment potency.

G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker. The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

I. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

J. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Active/Passive Immunization and Treatment/Prevention of Norovirus Infection

A. Formulation and Administration

The present disclosure provides pharmaceutical compositions comprising anti-norovirus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of norovirus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, by nebulizer, or via intrarectal or vaginal delivery. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

C. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### IV. Antibody Conjugates

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium⁹⁹m and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNC12, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. Immunodetection Methods

In still further embodiments, the present disclosure concerns immunodetection methods for binding, purifying, removing, quantifying and otherwise generally detecting norovirus and its associated antigens. While such methods can be applied in a traditional sense, another use will be in quality control and monitoring of vaccine and other virus stocks, where antibodies according to the present disclosure can be used to assess the amount or integrity (i.e., long term stability) of antigens in viruses. Alternatively, the methods may be used to screen various antibodies for appropriate/desired reactivity profiles.

Other immunodetection methods include specific assays for determining the presence of norovirus in a subject. A wide variety of assay formats are contemplated, but specifically those that would be used to detect norovirus in a fluid obtained from a subject, such as saliva, blood, plasma, sputum, semen or urine. In particular, semen has been demonstrated as a viable sample for detecting norovirus (Purpura et al., 2016; Mansuy et al., 2016; Barzon et al., 2016; Gornet et al., 2016; Duffy et al., 2009; CDC, 2016; Halfon et al., 2010; Elder et al. 2005). The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoas say (RIA), immunoradiometric assay, fluoroimmunoassay, chemiluminescent assay, bioluminescent assay, and Western blot to mention a few. In particular, a competitive assay for the detection and quantitation of norovirus antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing norovirus, and contacting the sample with a first antibody in accordance with the present disclosure, as the case may be, under conditions effective to allow the formation of immunocomplexes.

These methods include methods for purifying norovirus or related antigens from a sample. The antibody will preferably be linked to a solid support, such as in the form of a column matrix, and the sample suspected of containing the norovirus or antigenic component will be applied to the immobilized antibody. The unwanted components will be washed from the column, leaving the norovirus antigen immunocomplexed to the immobilized antibody, which is then collected by removing the organism or antigen from the column.

The immunobinding methods also include methods for detecting and quantifying the amount of norovirus or related components in a sample and the detection and quantification of any immune complexes formed during the binding process. Here, one would obtain a sample suspected of containing norovirus or its antigens and contact the sample with an antibody that binds norovirus or components thereof, followed by detecting and quantifying the amount of immune complexes formed under the specific conditions. In terms of antigen detection, the biological sample analyzed may be any sample that is suspected of containing norovirus or norovirus antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to norovirus or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used. In one exemplary ELISA, the antibodies of the disclosure are immobilized onto a selected surface exhibiting protein affinity, such as a well in a polystyrene microtiter plate. Then, a test composition suspected of containing the norovirus or norovirus antigen is added to the wells. After binding and washing to remove non-specifically bound immune complexes, the bound antigen may be detected. Detection may be achieved by the addition of another anti-norovirus antibody that is linked to a detectable label. This type of ELISA is a simple “sandwich ELISA.” Detection may also be achieved by the addition of a second anti-norovirus antibody, followed by the addition of a third antibody that has binding affinity for the second antibody, with the third antibody being linked to a detectable label.

In another exemplary ELISA, the samples suspected of containing the norovirus or norovirus antigen are immobilized onto the well surface and then contacted with the anti-norovirus antibodies of the disclosure. After binding and washing to remove non-specifically bound immune complexes, the bound anti-norovirus antibodies are detected. Where the initial anti-norovirus antibodies are linked to a detectable label, the immune complexes may be detected directly. Again, the immune complexes may be detected using a second antibody that has binding affinity for the first anti-norovirus antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of norovirus antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled norovirus monoclonal antibodies to determine the amount of norovirus antibodies in a sample. The basic format would include contacting a known amount of norovirus monoclonal antibody (linked to a detectable label) with norovirus antigen or particle. The norovirus antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

E. Immunodetection Kits

In still further embodiments, the present disclosure concerns immunodetection kits for use with the immunodetection methods described above. As the antibodies may be used to detect norovirus or norovirus antigens, the antibodies may be included in the kit. The immunodetection kits will thus comprise, in suitable container means, a first antibody that binds to norovirus or norovirus antigen, and optionally an immunodetection reagent.

In certain embodiments, the norovirus antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the norovirus or norovirus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

F. Vaccine and Antigen Quality Control Assays

The present disclosure also contemplates the use of antibodies and antibody fragments as described herein for use in assessing the antigenic integrity of a viral antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and continuous technical advances in the field offer a promise of developing potent new weapons against the oldest public health threats, as well as new ones—malaria, pandemic influenza, and HIV, to name a few—but also put a great pressure on manufacturers, regulatory authorities, and the wider medical community to ensure that products continue to meet the highest standards of quality attainable.

Thus, one may obtain an antigen or vaccine from any source or at any point during a manufacturing process. The quality control processes may therefore begin with preparing a sample for an immunoassay that identifies binding of an antibody or fragment disclosed herein to a viral antigen. Such immunoassays are disclosed elsewhere in this document, and any of these may be used to assess the structural/antigenic integrity of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

Antibodies and fragments thereof as described in the present disclosure may also be used in a kit for monitoring the efficacy of vaccination procedures by detecting the presence of protective norovirus antibodies. Antibodies, antibody fragment, or variants and derivatives thereof, as described in the present disclosure may also be used in a kit for monitoring vaccine manufacture with the desired immunogenicity.

### VI. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Virus-like particles. GII.4 Sydney 2012 virus-like particles (VLPs), based on strain AFV08795.1, were produced and purified, as previously described (Sapparapu et al., 2016). Briefly, VP1 and VP2 capsid protein sequences were cloned into the transfer vector pVL1392 (Epoch Life Sciences, Inc.). The vector was co-transfected with a bacmid vector into Sf9 insect cells. Recombinant virus then was used to inoculate Sf9 cells. VLPs were purified from the culture supernatant using a cesium chloride cushion gradient. GII.4 VLP assembly was verified visually using electron microscopy, and antigenicity was tested by western blot.

VLP binding assay. Antibody reactivity to GII.4 VLPs was tested using an indirect enzyme-linked immunosorbent assay (ELISA). Microtiter plates were coated with 1 μg/mL of GII.4 VLPs in PBS at 4° C. overnight. Wells then were blocked with 5% nonfat dry milk in PBS with 0.05% Tween-20 for 1 hour at room temperature. Purified antibodies were diluted serially in PBS and added to VLP-coated plates for 1 hour at room temperature. Microtiter plates were washed 3 times with PBS-0.05% Tween-20 in between each step. Antibodies that bound to VLPs were detected using horseradish peroxidase tagged anti-κ or -λ chain secondary antibodies (Southern Biotech) for 1 hour at room temperature. Plates were developed using the ultra-TMB reagent (Pierce ThermoFisher) and stopped using sulfuric acid. Absorbance was measured at 450 nm using a BioTek Synergy HT Microplate Reader.

VLP-carbohydrate binding antibody blockade assay. Microtiter plates were coated with 10 μg/mL of pig gastric mucin (PGM) Type III (Sigma) in PBS for 4 hours at room temperature, and then were blocked overnight at 4° C. in 5% nonfat dry milk in PBS with 0.05% Tween-20. GII.4 Sydney 2012 VLPs (0.5 μg/mL) were pretreated with each mAb applied in serial 3-fold dilutions with decreasing concentrations. Complexes then were applied to PGM-coated plates for 1 hour at room temperature. Microtiter plates were washed 3 times with PBS-0.05% Tween-20 in between each step. Bound VLPs were detected using guinea pig serum containing anti-GII.4 Sydney 2012 polyclonal antibodies, followed by an alkaline phosphatase-conjugated anti-guinea pig IgG. Optical density was measured at 405 nm using a Synergy HT Microplate Reader (BioTek).

Hemagglutination inhibition assay. Human type O+ red blood cells were purchased from Rockland Immunochemicals, Inc. Cells were pelleted at 500×g for 10 minutes at 4° C. and washed twice with PBS without Ca²⁺ or Mg²⁺. GII.4 Sydney 2012 VLPs (3.5 μg/mL) were pretreated with decreasing concentrations of each mAb, from 15 to 0.007 μg/mL, in PBS pH 5.5 and incubated at room temperature for 30 minutes. VLP-mAb complexes were added to an equal volume of 0.5% washed red blood cells in PBS pH 6.2 and incubated for 2 hours at 4° C. in a 96-well V-bottom microtiter plate. The HAI titer was determined as the lowest concentration of antibody that completely inhibited hemagglutination.

Human subjects. The inventor studied otherwise healthy adult subjects with a history of acute gastroenteritis contracted during a HuNoV outbreak in North Carolina between Feb. 27 and Mar. 1, 2013. The cause of the outbreak was determined by the Orange County, N.C. health department to be a GII.4 Sydney 2012 norovirus strain. Subjects were recruited after recovery to donate a one-time peripheral blood sample. The research study was approved by the Vanderbilt University Medical Center Institutional Review Board; all subjects provided written informed consent prior to participation.

Peripheral blood mononuclear cell (PBMC) isolation and hybridoma generation. The inventor obtained PBMCs from heparinized blood by density gradient centrifugation using Ficoll-Histopaque from 7 donors who had recovered recently from natural infection with HuNoV. B cells were transformed with Epstein Barr virus substrain B95.8 in the presence of 2.5 μg/mL of CpG10103, 10 μg/mL of cyclosporine A, and 10 μM Chk2 inhibitor. Approximately 10⁷ PBMCs were plated into a 384-well plate in transformation medium, and a week later were expanded into four 96-well plates containing irradiated human PBMCs as a feeder layer. After an additional 7 days of culture, the supernatants were screened by indirect ELISA for the presence of antibodies that bound to GII.4 Sydney 2012 VLPs. Antibodies that bound to GII.4 Sydney 2012 VLPs were detected using horseradish peroxidase tagged anti-human IgA or IgG secondary antibodies (Southern Biotech). Wells containing transformed B cells secreting anti-GII.4 Sydney 2012 VLP antibodies were fused with HMMA2.5 myeloma cells using a CytoPulse Sciences Generator. After fusion, hybridomas were plated in selection medium containing 100 μM hypoxanthine, 0.4 μM aminopterin, 16 μM thymidine and 7 μg/mL ouabain. After two weeks, hybridomas were screened for production of human antibodies reacting with GII.4 Sydney 2012 VLPs and then cloned biologically using single cell sorting on a FACSAria III flow cytometer in the Vanderbilt Flow Cytometry Shared Resource.

Competition-binding assay. To identify groups of antibodies binding to similar antigenic sites on norovirus GII.4 Sydney 2012, the inventor performed biolayer interferometry using an Octet® Red96 or Octet® HTX biosensor system (FortéBio). The Octet® HTX is a high-throughput biosensor system that was used to validate results obtained from the Octet® Red96 system. With both biosensor systems, antibodies and antigen were diluted in 1× kinetic buffer (FortéBio 18-5032). Glutathione S-transferase (GST)-tagged GII.4 Sydney 2012 P domain dimers were immobilized onto anti-GST biosensor tips (FortéBio 18-5096). The P domain dimers were coated onto the biosensor tip by immersing the tip in a solution containing dimers at a concentration of 5 μg/mL. The biosensor tip with the bound P domains was washed and then submerged into a well containing 50 μg/mL of the first antibody and then dipped into another well containing 50 μg/mL of the second antibody. If binding of the first antibody still resulted in greater than 66% of binding of the second antibody, the result was interpreted to be no competition. If binding of the second antibody was between 34 and 66% in the presence of the first antibody, there is believed to be partial competition. If 33% or less binding of the second antibody was noted in the presence of the first, both antibodies are believed to be in competition with each other. Antibodies then were clustered based on their binding patterns.

Stool filtrates. To prepare 10% stool filtrates, 4.5 mL of sterile PBS was added to 0.5 g of GII.4 Sydney 2012 positive stool sample. The stool suspension was sonicated using a cup horn sonicator and centrifuged at 1,500×g for 10 minutes at 4° C. Supernatant was collected and transferred to a new tube and centrifuged once again at 1,500×g, for 10 minutes at 4° C. The resulting supernatant then was passed serially through 5 μm, 1.2 μm, 0.8 μm, 0.45 μm and 0.22 μm filters, and aliquoted and frozen at −80° C.

Expression and purification of GST-GII.4 Sydney 2012 P domain. P1 and P2 domain sequences of GII.4 Sydney 2012, AFV08795.1, VP1 were cloned into the pGEX-4T-1 expression vector with a glutathione S-transferase (GST) tag and thrombin cleavage site. The P domain was expressed in Escherichia coli BL-21 cells and purified using standard column chromatography techniques with a prepacked Glutathione Sepharose Fast Flow column (GE Healthcare). GST-tagged proteins were eluted using 50 mM Tris-HCl, 10 mM reduced glutathione, pH 8.0 and stored at 4° C.

GII.4 Sydney 2012 virus neutralization assay. Human intestinal enteroids (HIEs) were generated and cultured as described previously (Ettayebi et al., 2016). Briefly, HIEs were grown as three-dimensional cultures in Matrigel (Corning) for 5 days and then plated as cell monolayer cultures in 96-well plates. Before plating, 96-well plates were pre-coated with collagen IV (Sigma) at 33 μg/mL in sterile cold water for 1.5 hours at 37° C. Three-dimensional HIEs were collected in 0.5 mM ethylenediaminetetraacetic acid diluted in ice-cold Dulbecco's PBS, no calcium, no magnesium (Life Technologies, Cat #14190-144) and spun down at 200×g for 5 minutes at 4° C. in a swinging bucket rotor. The pellet then was suspended in 0.05% trypsin/0.5 mL ethylenediaminetetraacetic acid and incubated at 37° C. for 4 minutes. Trypsin then was inactivated with complete medium without growth factors [CMGF(−)] supplemented with 10% fetal bovine serum (FBS). The resulting pellet was suspended and passed through a 0.4 μm cell strainer and spun down at 400×g at room temperature for 5 minutes. The pellet then was suspended in complete medium with growth factors [CMGF(+)] containing 10 μM Y-27632 (Sigma-Aldrich; Y0503) and seeded into a 96-well plate. After 24 hours, the culture medium was removed and replaced with differentiation medium. Cells were differentiated for 5 days. HuNoV GII.4 Sydney 2012 (TCH12-580) (Ettayebi et al., 2016) stool filtrate (2×10⁷ genome equivalents/μL) was used to test neutralization. Serial dilutions of the mAbs were prepared in CMGF(−) medium and each dilution was pre-incubated with 2.5×10⁵ genome equivalents of GII.4 Sydney 2012 at 37° C. for 1 hour. Samples were diluted with equal volume of CMGF(−) medium supplemented with 1000 μM sodium glycochenodeoxycholate. Monolayers then were inoculated with pre-incubated samples. At 1-hour post-infection (HPI), monolayers were washed twice and incubated with differentiation medium supplemented with 500 μM glycochenodeoxycholate. After 1 and 24 HPI, cells and medium were collected and RNA was extracted using KingFisher Flex Purification System and MagMax Viral RNA Isolation kit. For RT-qPCR, a primer pair (COG2R/QNIF2d) and probe (QNIFS) (Loisy et al., 2005; Kageyama et al., 2003) were used with qScript XLT One-Step RT-qPCR ToughMix reagent with ROX (Quanta Biosciences). Reactions were performed on an Applied Biosystem StepOne Plus thermocycler. A recombinant HuNoV RNA transcript was used to create a standard curve to quantitate viral genome equivalents in new RNA samples.

### Example 2—Results

Isolation of GII.4 VLP-reactive human mAbs. The first step here was to isolate naturally occurring human mAbs to GII.4 Sydney 2012 virus capsid protein from human subjects with prior GII.4 Sydney 2012 virus infection. The inventor used PBMCs collected from subjects with previous history of laboratory-confirmed GII.4 Sydney 2012 virus infection to generate human hybridoma cell lines secreting GII.4 VLP-reactive human mAbs. PBMCs were transformed with EBV and supernatants then were collected from lymphoblastoid cell lines and screened by ELISA for binding to GII.4 Sydney 2012 VLPs. Recombinant expression of norovirus genome ORF2 and ORF3 in a baculovirus expression system were used to generate VLPs that are antigenically and morphologically indistinguishable from native virions (Jiang et al., 1992). Antibodies that bound to VLPs were detected using horseradish peroxidase conjugated anti-λ or -κ light chain secondary antibodies. The inventor used an anti-light chain secondary antibody for detection in order to isolate antibodies of varying Ig heavy chain isotypes. Previous studies have noted the presence of diverse isotypes in the human polyclonal antibody response to infection, including an increase in IgA, IgG, and IgM antibodies in serum (Iritani et al., 2007; Gray et al., 1994), and the inventor has shown previously that human IgAs can be more potent than IgGs in blocking GI.1 VLPs from binding to histo-blood group antigens (HBGAs) (Sapparapu et al., 2016). Transformed B cell lines corresponding to supernatants that contained antibodies that bound to GII.4 VLPs were fused with myeloma cells to create human mAb-secreting hybridoma cells. The inventor isolated a panel of 25 hybridomas secreting VLP-reactive antibodies (21 IgGs and 4 IgAs) from 7 different donors (Table A and Supplementary Table 1).

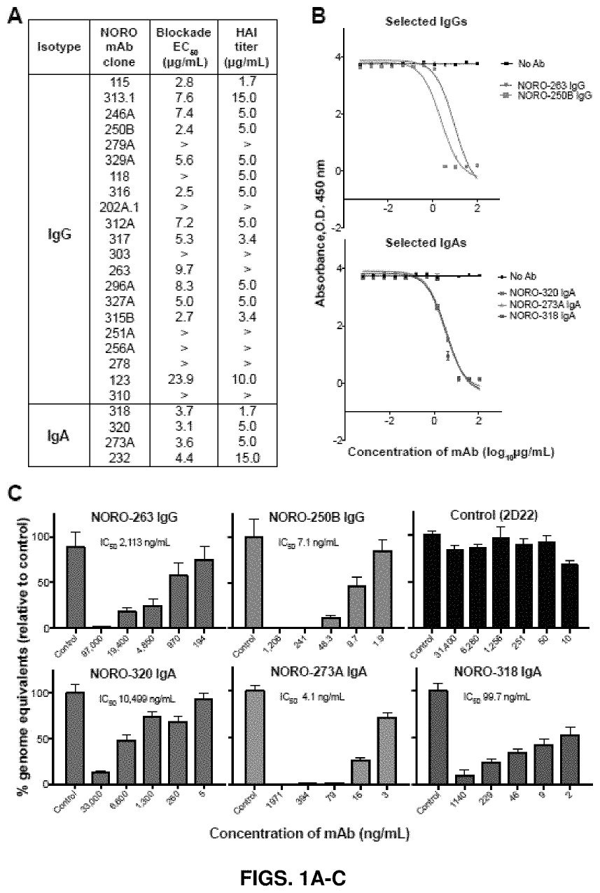

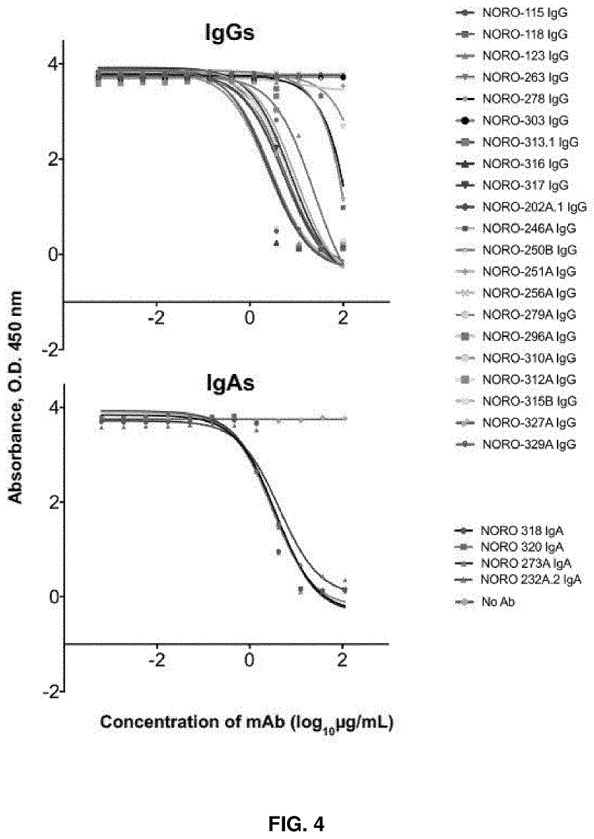

MAb binding and blockade of GII.4 VLPs. Next, the inventor sought to determine how well the antibodies bound to GII.4 Sydney 2012 antigens, and to identify if any blocked attachment of VLPs to surrogate receptor molecules. Half-maximal effective concentrations (EC₅₀) were determined for the panel of GII.4 Sydney 2012 VLPs reactive mAbs using indirect ELISA. For the 21 isolated IgGs, EC₅₀ values ranged from 0.1 to 6.2 μg/mL (Table A). For the 4 isolated IgAs, EC₅₀ values ranged from 0.1 to 0.4 μg/mL. The inventor initially used a surrogate system to assess the neutralizing capacity of all 25 mAbs. The presence of antibodies that block VLPs from binding to HBGAs in vitro correlates with protection against clinical NoV gastroenteritis (Atmar et al., 2015; Czako et al., 2015). To test blockade potential, serial dilutions of isolated mAbs were pre-incubated with GII.4 VLPs, and then VLP-antibody complexes were added to microtiter plates that had been coated previously with porcine gastric mucin type III (PGM) (Reeck et al., 2012). Previous studies have validated PGM as a reliable substrate to be used in VLP blockade assays (Lindesmith et al., 2012a). The inventor then determined EC₅₀ values for the 4 IgAs and 13 IgGs, which ranged between 2.4 to 23.9 μg/mL (FIGS. 1A-B, and FIG. 4). Blockade activity was not detected for 8 of the IgGs when using antibody concentrations as high as 100 μg/mL. The inventor determined the antibody variable gene heavy and light chain sequences for 17 of the 25 isolated mAbs (Supplementary Table 1). All 17 mAbs, both mAbs that did and did not inhibit GII.4 VLP binding to PGM, had distinct variable gene sequences, suggesting that blockade response is not restricted to a specific genetic sequence motif in antibody repertoires.

Hemagglutination inhibition (HAI) assay confirms mAb blockade activity. The inventor used a second functional assay to confirm the activity he observed in the blockade assay above. Previous studies have shown that an additional surrogate system to determine mAb neutralization is HAI, and that HAI serum antibody levels correlate with protection from symptomatic infection (Czako et al., 2012; Lindesmith et al., 2012a). The inventor used serial dilutions of the isolated mAbs and pre-incubated them with GII.4 Sydney 2012 VLPs. VLP-antibody complexes were then added to human type O+ RBCs. HAI activity was assessed visually, and HAI titers were determined (FIG. 1A). The majority of the mAbs, about 84%, had HAI activity similar to that of the measured blockade activity. Four mAbs differed in these measures, having either a greater than 2-fold difference in activities or exhibiting only HAI activity or only blockade activity.

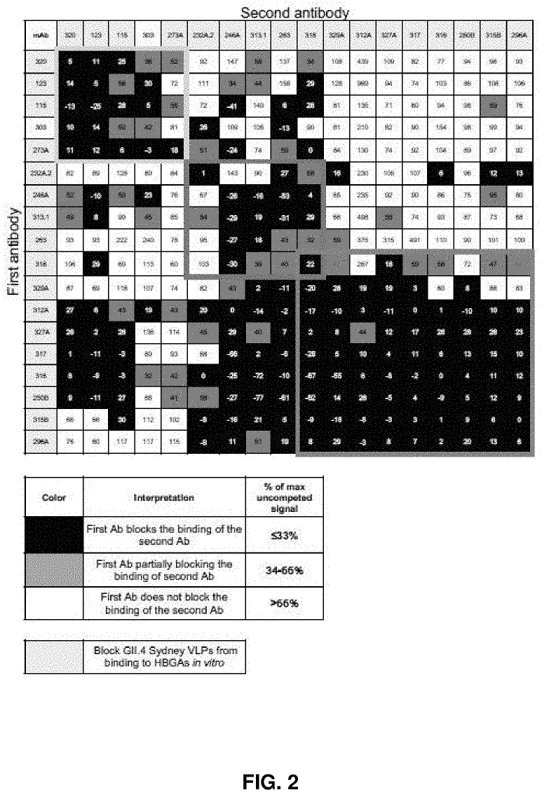

Neutralization assay using stem-cell derived enteroids. Inhibition of replication of GII.4 Sydney 2012 virus by mAbs NORO-250B, -263, -320, -273A, -318 and a non-GII.4 VLP binding control antibody, 2D22, were tested using an intestinal epithelial stem-cell derived in vitro cultivation system. The inventor selected two IgGs, NORO-263 and -250B and three IgAs, NORO-320, -273A and -318. These five mAbs were chosen to test neutralization by representative mAbs belonging to the three major antigenic sites identified on the GII.4 Sydney 2012 P domain and to test at least two mAbs belonging to each isotype (FIG. 2). Neutralization was measured by comparing the percent reduction of genome equivalents when compared to a no-antibody control within each assay using RT-qPCR (FIG. 1C). A no-antibody control was used in each assay to normalize for any variability between experiments. Variability was noted due to the high sensitivity when using genome equivalents to measure replication. To account for these differences, the inventor used six replicates for each mAb concentration tested within each assay and for the no-antibody control. He then averaged the genome equivalents from two separate assays for each antibody to obtain an IC₅₀ value. To verify that equal amounts of virus were added to each monolayer, each assay was performed in duplicate and RNA was collected from one assay at 1 HPI and from the other at 24 HPI. Four of the five antibodies tested, NORO-250B, -263, -273A and -318, had approximately 5 to 883-fold lower IC₅₀ values compared to blockade EC₅₀ values, or between 17 and 1,227-fold lower than HAI titers (FIGS. 1A and 1C). NORO-320 had a higher IC₅₀ in comparison to its blockade EC₅₀ or HAI titer. The dengue virus mAb 2D22 used as a similarly prepared negative control did not exhibit concentration-dependent inhibit of replication of GII.4 Sydney 2012 virus. Previous studies with polyclonal serum have shown that neutralization IC₅₀ values of GII.4 and GII.3 noroviruses are lower in comparison to blockade EC₅₀ values (Ettayebi et al., 2016). These studies as well as these data suggests that the HIE neutralization assay is likely more sensitive than the HBGA blockade or HAI assays.

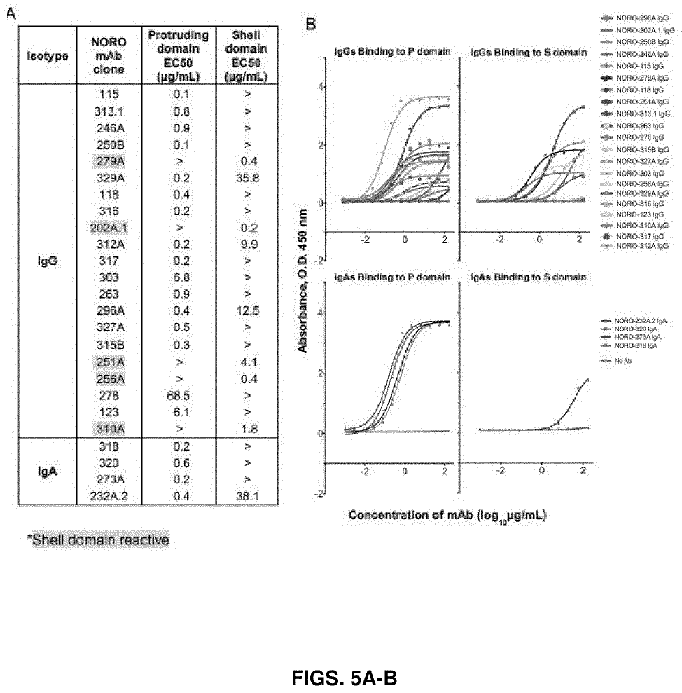

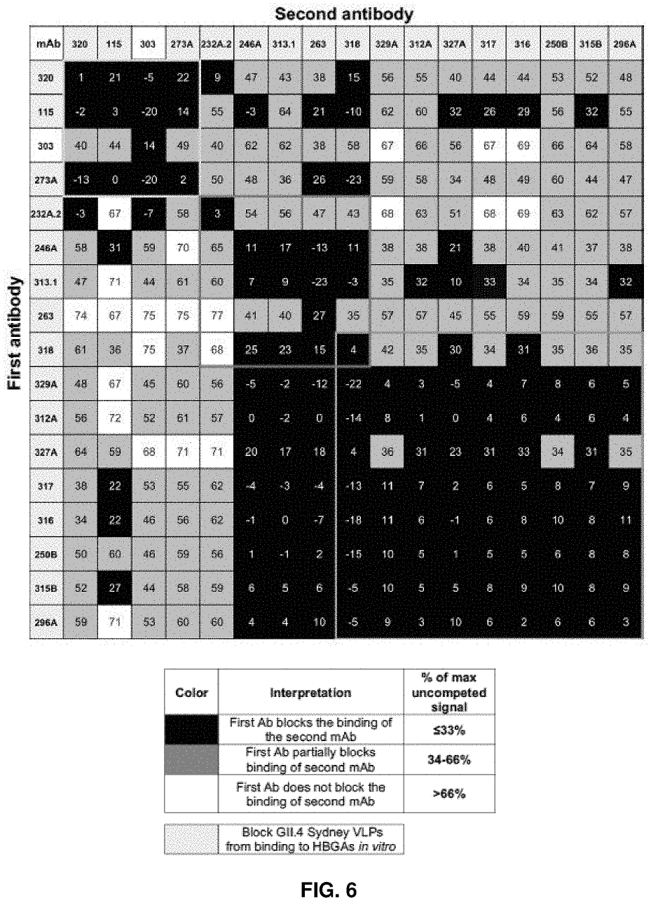

Binding studies using GII.4 Sydney 2012 protruding (P) domain dimers and shell (S) domain. The inventor next sought to determine if the antibodies were binding to the P or S domains of the major capsid protein VP1. Glutathione S-transferase (GST)-tagged recombinant GII.4 Sydney 2012 P domain dimers were expressed and purified using affinity column chromatography, as previously described (Choi et al., 2008). P domain dimers or recombinantly expressed S domains then were used as antigen for an indirect ELISA assay with serial dilutions of each mAb to determine if the 25 isolated mAbs were specific to the P or S domain of the GII.4 major capsid protein VP1. The VP1 region is divided into the S domain, which is not expressed on the recombinant GST-GII.4 P domain dimers, and the P1 and P2 subdomains that are expressed in the dimers. The P domain is the surface-exposed protruding region of the norovirus virion and also believed to include determinants for host cell attachment and antibody binding epitopes (Debbink et al., 2012; (Lindesmith et al., 2012b; Lochridge et al., 2005). The S domain is connected to the P domain by a flexible hinge region and forms the interior core of the viral capsid. The S domain has the highest degree of genetic sequence conservation of any protein domain in diverse norovirus strains (Parra et al., 2013). When binding was tested by ELISA, 20 of the 25 isolated mAbs bound to the GST-GII.4 P domain (FIGS. 5A-B). The five mAbs that did not bind to the P domain did bind to the S domain. NORO-329A, -312A, -296A and -232A.2, bound to both the P and S domain proteins, indicating that these antibodies likely bind to a quaternary epitope on the GII.4 Sydney 2012 major capsid protein. Isolated mAbs and GII.4 P domain dimers also were used for competition-binding studies. The inventor used a real-time biolayer interferometry biosensor system to identify potential major antigenic sites recognized by the GII.4 Sydney 2012 P domain binding mAbs. Neutralizing mAbs and mAbs that did or did not block GII.4 Sydney 2012 VLPs from binding to PGM were classified into three major competition-binding groups, with some overlap between two groups (FIG. 2). Despite multiple attempts, he was not able to detect binding using biolayer interferometry for 2 of the 20 mAbs that bound to the GII.4 Sydney 2012 P domain by ELISA. Competition-binding studies were performed using the Octet® RED96 and Octet® HTX systems, which are both instruments that can measure biomolecular interactions. The HTX system is a high-throughput system that has the ability to compete all 18 P domain binding mAbs within the same experiment. The RED96 system was only able to compete mAbs in groups of 8. Using data from the RED or HTX experiments, the inventor noted three major competition-binding groups on the GST-GII.4 Sydney 2012 P domain (FIG. 2 and FIG. 6). By using both instruments, in two independent laboratories, he was able to validate the reproducibility of the results.

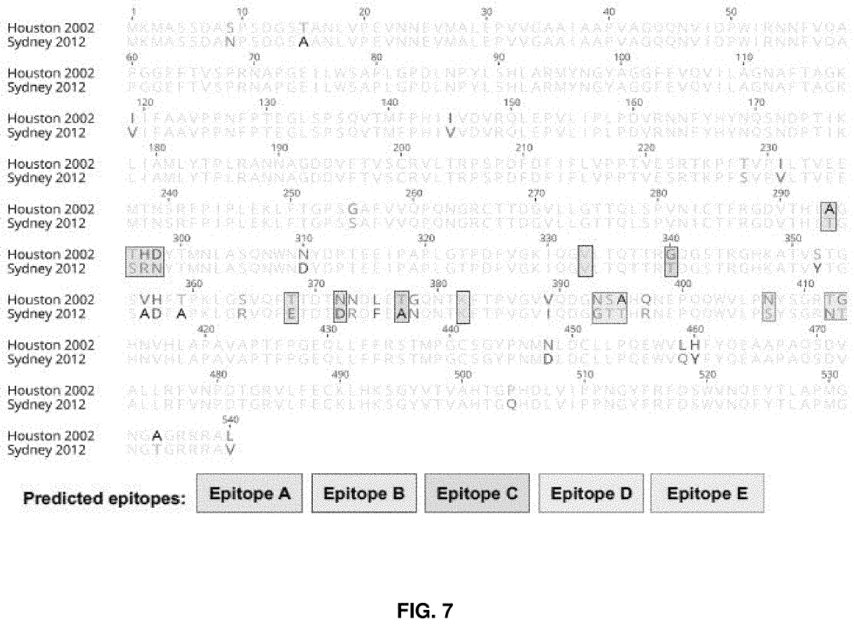

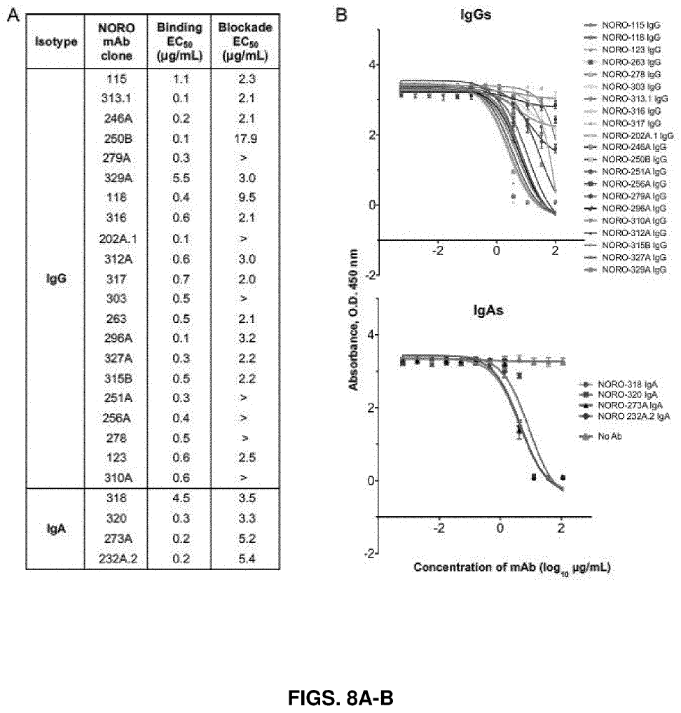

The emergence of new GII.4 strains has been associated with the evolution of the GII.4 major capsid protein and antigenic variation (Karst et al., 2015). To measure the reactivity of the inventor's panel of mAbs for another GII.4 strain, the inventor tested binding reactivity and blockade activity to a GII.4 Houston 2002 (ABY27560.1) variant. About 93% of the amino acid sequence of the major capsid protein is conserved between GII.4 Houston 2002 and Sydney 2012, but there are remarkable differences among predicted GII.4 blockade epitopes (FIG. 7). All 25 mAbs exhibited binding reactivity to GII.4 Houston 2002 antigen (FIGS. 8A-B). Only 3 mAbs, NORO-115, -329A, and -318 had a greater than 10-fold higher binding EC₅₀ value when compared to GII.4 Sydney 2012 VLPs (Table A). The 18 mAbs that either blocked with EC₅₀ values <100 μg/mL or had HAI titers <15 μg/mL all blocked GII.4 Houston 2002 VLPs from binding to PGM. GII.4 Houston 2002 and Sydney 2012 had different amino acid sequences in four of the five predicted blockade epitopes, these results suggest the potential existence of additional blockade epitopes or the use of Epitope B, which was conserved among both strains (FIG. 7).

### Example 3—Discussion

Here the inventor reports the first instance of neutralization of HuNoV by mAbs and describe a large panel of human mAbs that neutralize the pandemic GII.4 Sydney 2012 strain. Previously it was not possible to test norovirus neutralization directly because of the lack of a reliable in vitro culture system for norovirus replication. A surrogate system to predict neutralization was devised and used to study inhibition of the interaction between VLPs and HBGAs. The presence of blocking antibodies in serum correlates with protection from clinical gastroenteritis induced by HuNoV infections, and therefore the HBGA blocking assay has been considered a surrogate system for HuNoV neutralization (Reeck et al., 2010). Recently, the inventor developed an in vitro system using human enteroids to replicate multiple HuNoV strains¹³. Here, he used that system to identify the first norovirus human mAbs with demonstrated virus neutralizing activity. Of the 25 human mAbs isolated in this study, 17 of them blocked GII.4 Sydney 2012 VLPs from binding to PGM at concentrations as low as 2.4 μg/mL. The 18 mAbs that blocked GII.4 Houston 2002 from binding to PGM at concentrations as low as 2 μg/mL, also either blocked GII.4 Sydney 2012 VLPs from binding to PGM or inhibited hemagglutination at the concentrations tested. Interestingly, 13 of the 14 IgGs that blocked GII.4 Houston 2002 VLPs from binding to PGM did so at a lower EC₅₀ value in comparison to blockade EC₅₀ values for GII.4 Sydney 2012. This finding could indicate that donors had prior exposure to an earlier norovirus variant similar to GII.4 Houston 2002. This panel also contains the first reported human IgA mAbs that bind to GII.4 Sydney 2012 VLPs and also inhibit receptor binding. The inventor tested neutralization of live GII.4 Sydney 2012 HuNoV using the mAbs NORO-263, -320, -250B, -273A and -318. These antibodies were selected for testing so that he could investigate differences in neutralizing activity between mAbs of differing isotypes, those which belong to different competition-binding groups, and those with different binding and blockade EC₅₀ values. NORO-250B, -263, -273A and -318, had lower neutralization IC₅₀ values in comparison to blockade EC₅₀ values and HAI titers. Surprisingly, NORO-320 had an IC₅₀ value about 2-fold higher than its HAI titer and about 3-fold higher than its blockade EC₅₀ value. Previous studies have noted differences in blockade potency of GI.1 VLPs among human IgG and IgA mAbs with blockade potency being enhanced for IgAs (Sapparapu et al., 2016). To draw a similar conclusion for GII.4 Sydney 2012 neutralizing human antibodies, it would be essential to test mAbs binding to different epitopes with identical variable domain sequences and distinct isotypes. Such studies could determine if isotype plays a critical role in neutralization of GII.4 Sydney 2012 by human mAbs.

HuNoV-specific antibodies have been described previously, but these were antibody fragments derived from phage display libraries (Huang et al., 2014), murine mAbs from infected mice (Crawford et al., 2015), nanobodies from alpacas immunized with VLPs (Koromyslova and Hansman, 2017), or mAbs isolated from patient PBMCs with an unknown norovirus history of exposure (Lindesmith et al., 2012a). Such antibodies do not provide direct information about the physiologic human humoral immune response to HuNoV infection. The therapeutic potential of mouse mAbs is limited, since they have been shown to induce human anti-mouse antibody responses. There has been little progress in understanding individual HuNoV-specific antibodies in the past because of the difficulty in generating human mAbs with functional activity. Here the inventor used a hybridoma technology (Smith and Crowe, 2015) and circulating B cells from convalescent patients to produce human mAbs. This approach generates hybridoma cell lines from circulating B cells that express naturally occurring and matched heavy and light chain genes. An additional benefit of using this approach is that it does not involve the use of any laboratory animals to produce antibodies. Using this method, the inventor isolated 25 GII.4 Sydney 2012 VLP-reactive mAbs. The majority of these antibodies were neutralizing when tested in a surrogate system for neutralization and all five of the mAbs tested also inhibited replication of live GII.4 Sydney 2012 virus by direct neutralization in vitro. Neutralizing human mAbs have potential for use in prophylactic, therapeutic or diagnostic applications. The inventor currently does not have any drugs available to treat or prevent HuNoV infection, so the inventor's panel of neutralizing mAbs now have the high potential to impact the design of improved diagnostic and therapeutic measures for HuNoVs.

Since the mid-1990s, new antigenically diverse GII.4 pandemic viral strains have emerged continuously every 2 to 5 years, and today these strains continue to be the predominant cause of norovirus outbreaks. In 2012, the epidemic GII.4 Sydney variant emerged in Australia and began spreading globally. Even though blockade epitopes among some contemporary GII.4 strains have been predicted or identified, the inventor has limited information about the neutralization determinants on GII.4 Sydney 2012 viruses (Debbink et al., 2013; Lindesmith et al., 2012a). Here, the inventor determined that there are at least three major antigenic and neutralizing sites on the P domain of GII.4 Sydney 2012 viruses. In the future, defining neutralization epitopes in high resolution with neutralizing antibodies could contribute valuable insights for rational structure-based vaccine design efforts.

HuNoV is one of the leading causes of severe acute gastroenteritis, therefore the global burden of norovirus infection is extremely high in both developed and developing countries. Unfortunately, there is currently no licensed vaccine to prevent norovirus infection. Efforts to design a vaccine have been hindered by the lack of a small animal model or tissue culture model to test neutralization or infection, the antigenic heterogeneity among noroviruses, and uncertainty about the durability of protective immunity (Debbink et al., 2014). Vaccine efforts have focused on the use of monovalent GI.1 or bivalent GI.1/GII.4 virus-like particles or P particle subunits (El-Kamary et al., 2010; Bernstein et al., 2015; Tan et al., 2011). Clinical trials have shown that norovirus VLP vaccines are immunogenic and without frequent serious adverse events (Ramirez et al., 2012; Leroux-Roels et al., 2018). The inventor now has developed a reliable in vitro system to test the replication or inhibition of replication of live noroviruses. Mapping the neutralization or blockade epitopes using the panel of mAbs the inventor isolated against this circulating pandemic strain of norovirus will provide critical information that can be used for the design of future VLP vaccines that can elicit a protective immune response.

### Example 4—Materials and Methods

Generation of virus-like particles. Virus-like particles (VLPs) based on norovirus strains GI.1 (M87661), GI.2 (AF435807), GI.3 (AF439267), GII.3 (TCH02-104), GII.4 (AFV08795.1), GII.6 (AF414410), GII.13 (JN899242) and GII.17 (AB983218) were expressed recombinantly and purified as previously described (Jiang et al., 1992). The inventor used a baculovirus recombinant protein expression system for VLP production. The inventor cloned the VP1 and VP2, major and minor, protein capsid sequences from each strain into the transfer vector pVL1392 (Epoch Life Science, Inc). Sf9 insect cells were co-transfected with a transfer vector corresponding to a specific strain and with a bacmid vector. Recombinant baculovirus was isolated and expanded. VLPs were purified from cell culture supernatants using a sucrose and cesium chloride gradient. VLP formation was verified using electron microscopy.

Reactivity to VLPs by ELISA. An ELISA was used to testing binding of human mAbs to VLPs, as was previously described (Alvarado et al., 2018). Each VLP was coated individually at 1 μg/mL on 384-well microtiter plates at 4° C. overnight. Plates then were blocked for one hour at room temperature using 5% nonfat dry milk in PBS with 0.05% Tween-20. For screening and EC₅₀ analysis, antibody reactivity to VLPs was detected using horseradish peroxidase (HRP) tagged anti-κ or -λ chain secondary antibodies (Southern Biotech). 1-Step™ Ultra-TMB Substrate Solution (Pierce ThermoFisher) was used to detect HRP activity.

Human subjects. The Vanderbilt University Medical Center Institutional Review Board approved of the participation of the 6 adult subjects used in this study. All participants provided written informed consent before the inventor obtained blood samples. The subjects were healthy with a previous history of acute gastroenteritis.

Human hybridoma generation. Human hybridomas secreting human mAbs were generated as previously described (Alvarado et al., 2018). Briefly, PBMCs were isolated from human subject blood samples using Ficoll-Histopaque and density gradient centrifugation and then cryopreserved. Later, cells were thawed, transformed using Epstein-Barr virus, CpG10103, cyclosporine A and a Chk2 inhibitor and plated in a 384-well plate. Transformed cells were incubated at 37° C. for 7 days, and then expanded into 96-well plates containing irradiated human PBMCs. Four days later, cell supernatants were screened by indirect ELISA for the presence of anti-norovirus VLP cross-reactive mAbs. B cells secreting cross-reactive mAbs were electrofused to HMMA2.5 myeloma cells and plated in medium containing hypoxanthine, aminopterin, thymidine and ouabain. Hybridoma cell lines were incubated at 37° C. for 14 days, and then supernatants were screened by indirect ELISA for productions of cross-reactive mAbs. Cell lines expressing cross-reactive mAbs then were cloned biologically using single-cell fluorescence-activated cell sorting.

Purification of cross-reactive mAbs. After cloning, hybridoma cell lines producing cross-reactive mAbs were expanded gradually from 48-well plates to 12-well plates, T-25, T-75 and eventually to four T-225 flasks for each cell line. Supernatant from each cell line also was screened by ELISA to determine the corresponding light chain for each clone. Following 4-weeks of incubation at 37° C., supernatant from the four T-225 flasks was harvested and filtered through a 0.4-μm filter. The supernatant was filtered using column chromatography, specifically HiTrap KappaSelect and Lambda FabSelect affinity resins (GE Healthcare Life Sciences).

VLP-carbohydrate mAb blockade assay. To test the ability of each mAb to inhibit the interaction between the selected VLPs and glycans in vitro, the inventor used a blockade assay. As previously described, the inventor coated microtiter plates with 10 μg/mL of pig gastric mucin Type III (Sigma) for 4 hours at room temperature. Plates then were blocked overnight at 4° C. in 5% nonfat dry milk. VLPs at 0.5 μg/mL were pretreated with serially diluted concentrations of each mAb for 1 hour at room temperature. VLP-mAb complexes were added to the PGM-coated and blocked microtiter plates. After 1 hour of incubation, the plates were washed 3 times with PBST and the same was done in between each step. Bound VLPs were tested using murine serum containing anti-GI.3, GII.4, GII.6 or GII.17 polyclonal antibodies, followed by an HRP conjugated goat anti-mouse IgG human adsorbed antibody. Optical density was measured at 450 nm using a Synergy HT Microplate Reader (BioTek).

Expression and purification of protruding and shell domain for selected NoV strains to be used in Ab binding studies. In order to map the epitope of cross-reactive mAbs, the inventor first recombinantly expressed P1 and P2 domain sequences or shell domain of GI.3 (AF439267), GII.4 (AFV08795.1), GII.6 (AF414410), GII.13 or GII.17 (AB983218). P domain sequences were cloned into the pGEX-4T-1 expression vector with a GST tag and thrombin cleavage site. The P domain then was expressed in Escherichia coli BL-21 cells and purified using a Glutathione Sepharose Fast Flow Column (GE Healthcare) and column chromatography. The S domain sequences were cloned into pVL1392 and co-transfected with a bacmid vector into Sf9 insect cells. Recombinant baculovirus particles then were harvested and used to inoculate Sf9 cells. S domain particles were then purified from the inoculated Sf9 cell culture supernatant using a sucrose and a cesium chloride cushion gradient.

Expression, purification and crystallization of GII.4 P domain and NORO-320 Fab. The sequence for the GII.4 protruding domain was cloned into the expression vector pMal-C2E (New England BioLabs). The expression vector includes a N-terminal His₆-maltose binding protein (MBP) tag and a tobacco etch virus (TEV) protease cleavage site between the MBP and P domain sequence. The P domain was expressed in E. coli BL21(DE3) and purified using an AffiPure Ni-NTA agarose bead column (GenDepot). The His-MBP tag was then removed using TEV protease and separated from the P domain by purifying it once again using His-Trap (GE Healthcare), MBPTrap (GE Healthcare) affinity columns and size exclusion chromatography. Finally, the purified P domain was concentrated and stored in 20 mM Tris-HCl buffer (pH 7.2) containing 150 mM NaCl, and 2.5 mM MgCl₂.

The nucleotide sequences of the variable domain of mAb NORO-320 was optimized for mammalian expression and synthesized (Genscript) for expression and purification of recombinant Fab. The heavy chain fragment was cloned into a vector for expression of recombinant human Fabs (McLean et al., 2000). The light chain was cloned into a vector for κ light chain. Each vector was transformed independently into E. coli cells, and DNA was then purified. Both the heavy and light chain encoding vectors were transfected into CHO cells using an ExpiCHO™ expression system. Cell supernatant was collected, centrifuged and filtered using a 0.45 μm filter. NORO-320 Fab was purified by affinity chromatography using a KappaSelect (GE Healthcare).

Purified GII.4 P domain and NORO-320 Fab were combined in a 1:1.5 molar ration and incubated for 1 hour at 4° C. The mixture was passed through an S75pg 16/60 gel filtration column, and the peak corresponding to the complex was collected. The size of the complex and presence of both proteins was validated on are SDS-PAGE gel. The peak fractions were then pooled and concentrated to 10 mg/mL for crystallization trials. Crystallization screening using hanging-drop vapor diffusion method at 20° C. was set up using a Mosquito nanoliter handling system (TTP LabTech) with commercially available crystal screens, and crystals were visualized by using a Rock Imager (Formulatrix). The GII.4 P domain-NORO-320 Fab complex crystallized in a buffer containing 0.1 M BIS-TRIS prop 8.5 pH, 0.2 M KSCN, 20% w/v PEG 3350. Crystals diffracted to 2.25 Å resolution.

Diffraction, data collection, and structure determination. X-ray diffraction data for the GII.4 Sydney 2012 P domain-Fab NORO-320 crystals were collected on the beamline 5.0.1 at Advanced Light Source (Berkeley, Calif.). Diffraction data were processed using HKL2000 (Z. Otwinowski and W. Minor, 1997). The previously published GII.4 (strain TCH05) P domain structure (PDB ID 3SJP) and the neutralizing Fab 5I2 (PDB ID 5KW9) were used as the search models by molecular replacement using program PHASER (McCory et al., 2007). Iterative cycles of refinement and further model building were carried out using PHENIX (Adams et al., 2010) and COOT programs (Emsley, P., and K. Cowtan, 2004). During the course of the refinement, and following the final refinement, the stereochemistry of the structures was checked using Molprobity (molprobity.biochem.duke.edu/). Data refinement and statistics are given in Table B. The interactions between P domain and the Fab for NORO-320 were analyzed using LigPlot+ v.2.1 (Laskowski et al., 2011). Figures were prepared using Chimera (Pettersen et al., 2004).

### Example 5—Results

Isolation of broadly binding anti-NoV human mAbs. To isolate cross-reactive NoV human mAbs, the inventor used EBV and additional B cell stimuli to transform memory B cells in PBMCs obtained from patients who were overall healthy but with a previous history of acute gastroenteritis, as previously described (Sapparapu et al., 2016). A week later, transformed PBMCs supernatants were tested by ELISA to screen for the expression of mAbs that bound to more than one representative strain NoV VLP. The VLPs used to screen were NoV GI.1, GI.2, GI.3, GII.3, GII.4, GII.6, GII.13 or GII.17. Each VLP was coated individually and blocked on a microtiter plate before the screening. The bound antibodies were detected using alkaline phosphatase conjugated goat anti-human κ or λ chain secondary antibodies to capture binding activity by any antibody isotype. Wells that contained transformed B cells expressing mAbs that recognized more than one VLP then were expanded. B cells secreting anti-NoV mAbs were rescued by hybridoma formation. Binding reactivity to pandemic GII.4 Sydney 2012 was previously characterized for 5 of the mAbs included in the inventor's panel (Alvarado et al., 2018). The heavy and light variable gene regions were sequenced for all 12 mAbs and the V, D, J and other variable gene sequence features were analyzed (data not shown) (Brochet et al., 2008). Each of the mAbs had unique variable gene sequences, suggesting that cross-reactivity was not limited to one antibody clonotype.

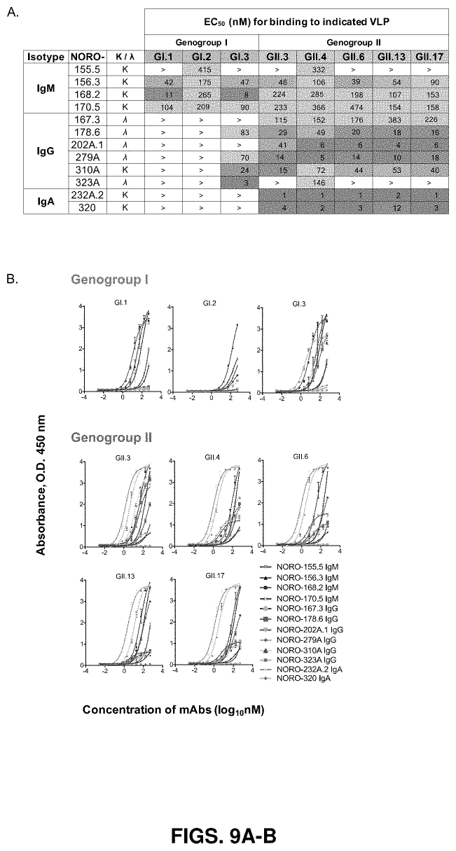

Binding and blockade activity of cross-reactive mAbs to NoV GI and GII VLPs. To assess binding reactivity and blockade function of the 12 mAbs, the inventor used indirect ELISA and a VLP blockade assay. The concentration of each mAb was normalized first for the number of antigen binding sites. The inventor then tested binding starting at a concentration of 500 nM, followed by 11 serial dilutions. Each concentration was tested in duplicate, and the complete experiment was repeated 3 times. The inventor used these data to determine the EC50 value of each mAb when binding to NoV GI.1, GI.2, GI.3, GII.3, GII.4, GII.6, GII.13 or GII.17 VLPs (FIGS. 9A-B). The inventor noticed 3 distinct binding patterns. NORO-168.2, -156.3 and -170.5, all IgMs, each exhibited wide breadth by binding to all VLPs tested. Both IgAs, NORO-232A.2 and -320, as well as NORO-167.3 and -202A.1, both IgGs, exhibited specificity of binding only for GII variants. The remaining mAbs, NORO-155.5, -178.6, -279A, -310A and -323A reacted with at least one GI and GII strain.

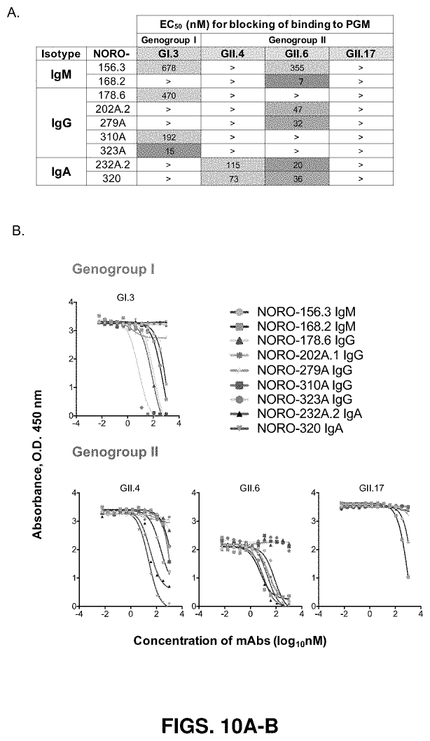

To determine if any of the isolated cross-reactive mAbs had functional activity, the inventor used a surrogate system to analyze neutralization. PGM purified from porcine stomach mucosa contains both H and Lewis antigens, α-1,2- fucose and α-1,4-fucose (Lindesmith et al. 2008; Lindesmith et al. 2012; Tian et al., 2010). The use of PGM in blockade assays has been validated previously (Lindesmith et al., 2012). He first tested if GI.1, GI.2, GI.3, GII.3, GII.4, GII.6, GII.13 or GII.17 VLPs could bind to the glycans present in PGM. The optimal concentrations of mAbs were normalized before testing blockade ability and tested at concentrations beginning at 1,000 nM and then diluted serially (FIGS. 10A-B). Blockade studies also were repeated three times. Only GI.3, GII.4, GII.6 and GII.17 bound to PGM. NORO-155.5 and -170.5, both IgMs, and -167.3, an IgG, did not inhibit GI.3, GII.4, GII.6 or GII.17 VLPs from binding to PGM in vitro. 9 of the 12 mAbs blocked at least 1 of the 4 VLPs tested. None of the 8 mAbs with binding reactivity to GII.17 VLPs had any GII.17 blockade activity. The absence of GII.17 blockade could be due to the broad binding HBGA spectrum of GII.17 NoVs and the glycan heterogeneity present in PGM.

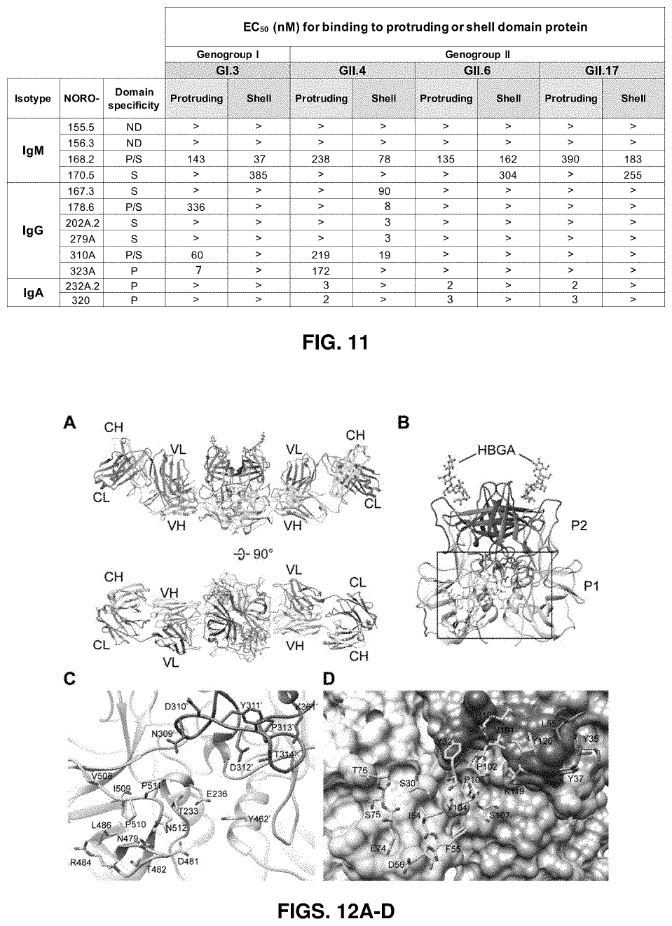

Binding to GI.3, GII.4, GII.6 or GII.17 variant protruding (P) vs shell (S) domains. The major capsid protein, VP1, which forms the icosahedral capsid, is divided into the protruding (P) and shell (S) domains (Prasad et al., 1996). To map where the cross-reactive mAbs bind, the inventor first recombinantly expressed and purified the S and P domains of GI.3, GIII.4, GII.6 and GII.17 NoV strains. Antibody binding to S, P and VLPs, containing both S and P, was tested and compared. His₆-MBP (maltose binding protein)-tagged recombinant P domains were expressed in E. coli and purified using affinity column chromatography, as previously described (Choi et al., 2008). S domain sequences were cloned into pVL1392, expressed using a baculovirus expression system and purified using a sucrose and cesium chloride cushion gradient. P and S domain recombinant proteins were coated at equal concentrations of 2 μg/mL on microtiter plates and blocked with 5% nonfat dry milk in 1×DPBS with 0.05% Tween-20. Before adding the mAbs to plates, each mAb was normalized according to the number of antigen binding sites. The inventor tested binding starting at a concentration of 500 nM followed by 11 serial dilutions of each mAb to obtain the half-maximal binding concentrations. Both IgAs, NORO-232A.2 and -320, appeared to bind specifically to the GII.4, GII.6 and GII.17 P domains (FIG. 11). Cross-reactive murine mAbs also have been mapped to the NoV P domain (Parker et al., 2005). NORO-168.2 bound to both the P and S domains of GI.3, GII.4, GII.6 and GII.17, but in all instances had a lower EC₅₀ value when bound to the S domain. Some mAbs like NORO-155.5 and -156.3 did not bind to any of the P or S domains tested. Loss of binding may suggest that these mAbs require both the S and P domain to be present for Ab binding.

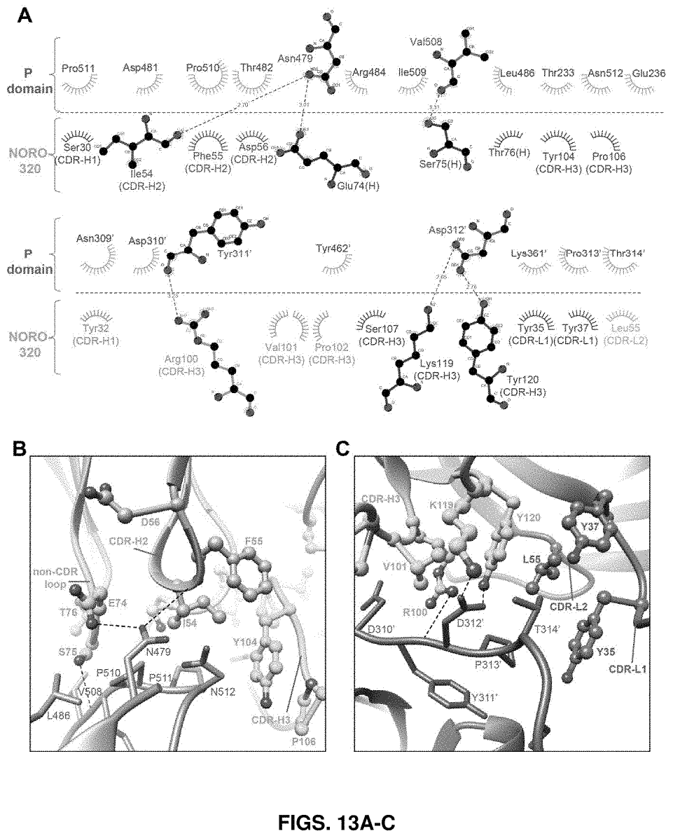

Structure of NORO-320 Fab in complex with GII.4 P domain. In previous studies, the inventor determined that NORO-320 not only neutralizes GII.4 Sydney 2012 using a surrogate neutralization assay, but also inhibits replication of infectious GII.4 Sydney 2012 virus when using a human intestinal enteroid culture (Alvarado et al., 2018). Here, the inventor discovered that NORO-320 binds broadly across selected GII strains and also blocks GII.6 from binding to PGM. To determine how NORO-320 binds so broadly and neutralizes diverse GII NoV strains, the inventor used X-ray crystallography to identify the structural basis for molecular recognition. The inventor obtained a 2.3 Å resolution structure of NORO-320 Fab in complex with the GII.4 P domain (FIGS. 12A-B). According to the structure, NORO-320 Fab binds perpendicular to the 2-fold axis of the P domain dimer near a region close to the shell domain. This finding shows that NORO-320 Fab does not bind directly or close to the HBGA binding site to inhibit GII.4 VLP-carbohydrate binding or replication of GII.4 Sydney 2012 virus. The inventor considered the possibility that NORO-320 Fab binding allosterically could inhibit HBGA binding by causing a conformational change in the glycan binding site. However, the structural superposition of the GII.4 P domain in complex with NORO-320 and that with the bound HBGA revealed that the P domain dimer structure remains invariant with an r.m.s.d of ˜1.1 Å for the matching Cα atoms. The inventor then hypothesized that neutralization of GII.4 by NORO-320 could be a result of steric hindrance since full-length NORO-320 is originally a dimeric IgA.

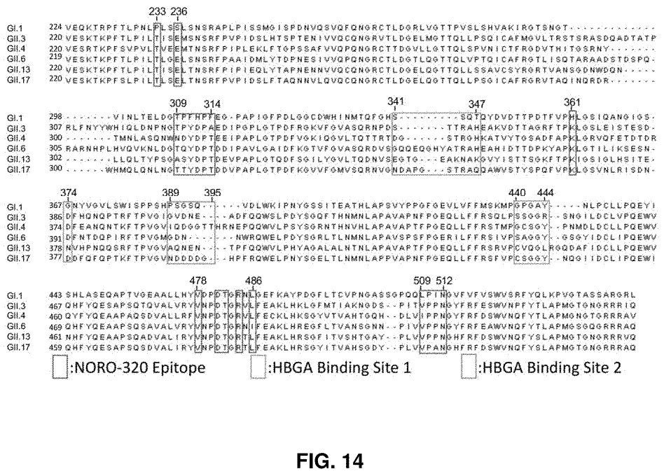

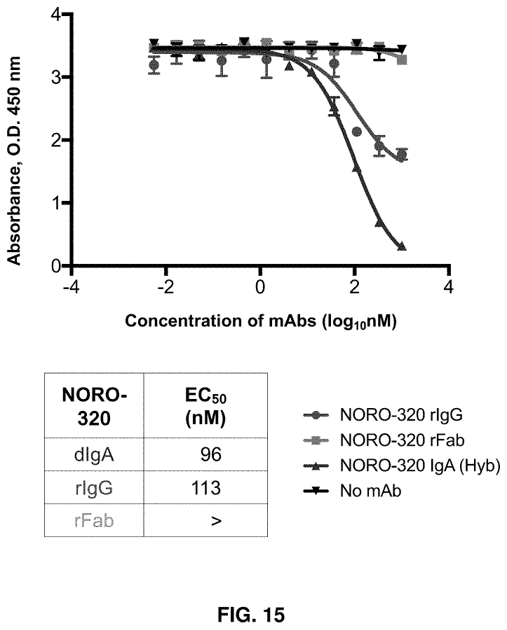

Molecular details of recognition of GII.4 P domain by NORO-320. The crystal structure of NORO-320 Fab-GII.4 P domain complex shows that the antibody makes extensive interactions with the P domain. The Fab binding site on the P domain is formed by residues from the P1 subdomain of one subunit and the P2′ subdomain of its dimeric partner (FIGS. 12C-D). The paratope in NORO-320 includes residues from the CDRs of both light and heavy chains. The Fab binding is stabilized by both hydrogen bond and hydrophobic interactions (FIG. 13A). For instance, the sidechain of N479 in the P1 subdomain hydrogen bonds with 154 from CDRH2 and E74 of a non-CDR loop, whereas residues L486, V508, P510, P511, and N512 are involved in hydrophobic interactions with residues from CDRH2 and CDRH3 (FIG. 13B). Residue D312 of the P2′ subdomain interact with K119 and Y120 of CDRH3, involving both direct hydrogen bond and hydrophobic interactions (FIG. 13C). In addition to CDRHs, three light chain residues Y35 and Y37 of CDRL1 and L55 of CDRL2 form hydrophobic interactions with P313′ and T314′ of the P1′ subdomain. To understand how NORO-320 can bind to VLPs of various GII strains, the inventor aligned the P domain amino acid sequences of GI.1, GII.3, GII.4, GII.6, GII.13 and GII.17 (FIG. 14). Sequence alignment revealed a 78 to 89% conservation at these sites among the GII strains. In contrast, the P domain sequence in the GI genogroup show significant changes in this region. The high level of sequence conservation could explain why NORO-320 binds broadly among GII.3, GII.4, GII.6, GII.13 and GII.17 strains.

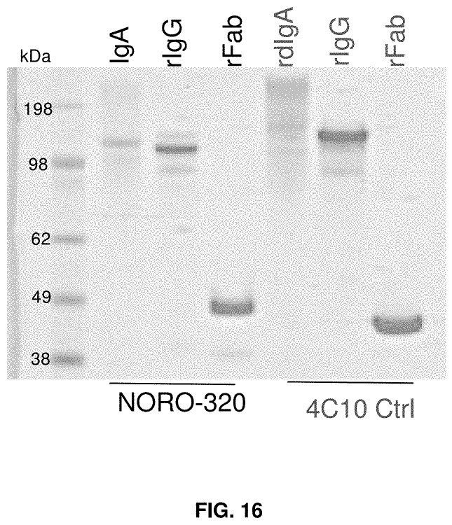

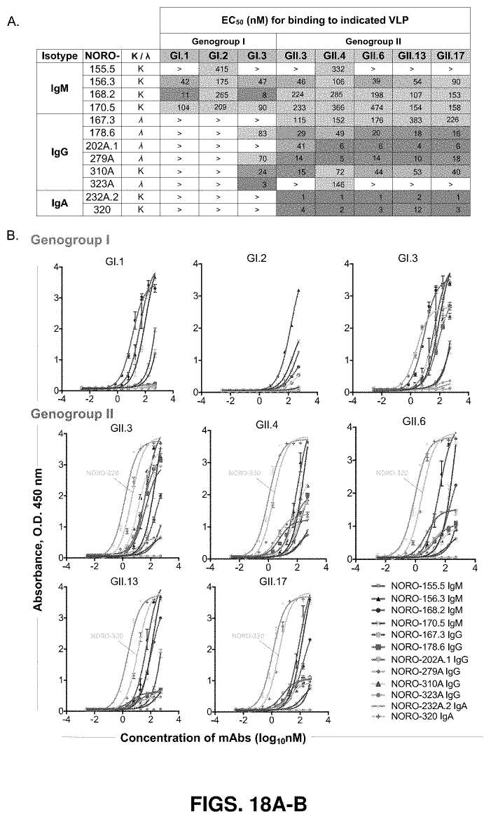

Mechanism of neutralization of NoV by broad human mAbs. To determine if NORO-320 neutralizes GII.4 because it sterically hinders NoV binding to glycans, the inventor tested if blockade activity was influenced by the molecular weight or size of NORO-320. In order to obtain varying forms of NORO-320, he recombinantly expressed the sequence of the variable domain in Fab or IgG heavy chain recombinant expression vectors. The light chain variable sequence also was cloned into a kappa light chain recombinant expression vector. Corresponding heavy and light chains were transfected using Chinese hamster ovary (CHO) cells in an ExpiCHO™ expression system. To verify the molecular weight of the original hybridoma-expressed IgG and each of the recombinantly expressed mAbs, 4 μg of each mAb, along with a set of control mAbs of known molecular weight, were resolved on an SDS-PAGE gel under non-reducing conditions (FIGS. 17A-D). All the mAbs were of the expected apparent molecular weight, dIgA˜350 kDa, IgG˜150 kDa and Fab˜50 kDa. The inventor then tested NORO-320 IgA, recombinant IgG, recombinant Fab or a no mAb control for their ability to inhibit GII.4 Sydney 2012 VLPs from binding to PGM in vitro. As hypothesized based on size, the large NORO-320 IgA had the lowest EC₅₀ value followed by NORO-320 recombinant IgG (FIG. 15). Recombinant NORO-320 Fab did not block GII.4 Sydney 2012 VLPs from binding to PGM at concentrations as high as 1,000 nM. This finding indicates that the dimeric NORO-320 likely neutralizes GII strains broadly because of the capacity of this large molecule to mediate steric hindrance to receptor binding (FIGS. 17A-D). The IgMs and IgGs that bind to the S domain of VP1 could potentially neutralize NoVs by disrupting the structural integrity of the virus particles. For example, the IgM NORO-168.2 broadly cross-reacts with GI and GII NoVs by binding to the P and S domains of GI.3, GII.4, GII.6 and GII.17 (FIG. 11). Further understanding of the diverse neutralization mechanisms for circulating human NoVs warrants future structural studies of these human mAbs (FIGS. 17A-D).

### Example 6—Discussion

Isolation of naturally occurring broad-spectrum human mAbs to NoV holds great promise for the discovery of new candidate therapeutics, as well as identifying critical epitopes for the rational design of new structure-based broadly protective NoV vaccines. In the past, the genetic and antigenic diversity across circulating strains of NoV has made the generation of a broadly immunogenic vaccine extremely difficult. The primary goal of this study was to define the molecular and structural determinants of cross-reactivity and blockade, or neutralization, using human mAbs to circulating strains of HuNoV. Previous studies have characterized the antigenic landscape of specific HuNoV strains, but with the rapid emergence of new genetically diverse strains, there is a need to map new immunogenic epitopes. This new information also can build upon previous studies to help track the evolution of HuNoVs (Lindesmith et al., 2013). Identification of antigenic epitopes using human mAbs also will provide insight into the immunogenicity of NoVs in humans.

Here, the inventor described the isolation of 12 anti-NoV cross-reactive human mAbs, 4 IgMs, 6 IgGs, and 2 IgAs, from subjects with a previous history of acute gastroenteritis. To determine the functional activity of the isolated mAbs, he used a previously validated surrogate blockade assay that measures the inhibition of VLPs from binding to glycans in vitro (Reeck et al., 2010). Not all of the strains tested bind to the same glycan, so the inventor was only able to test the inhibition of binding for NoV VLPs GI.3, GII.4, GII.6, and GII.17. Of the 12 mAbs isolated, 9 mAbs blocked at least 1 of VLPs tested from binding to PGM with EC₅₀ values less than 1 μM.

Prior to these studies, the inventor expected that cross-reactive and neutralizing human mAbs would bind predominantly to the P subdomain of the circulating strains, similar to the findings for cross-reactive murine Abs (Crawford et al., 2015). The P domain has more surface exposure on a live virion than the S domain and should, therefore, be more accessible than the S domain. Both previously characterized mAbs, the human IgA 5I2 and the mouse IgG 10E9, target regions adjacent or at the HBGA-binding site on the P2 subdomain thereby directly preventing the binding of glycans (Shanker et al., 2016; Koromyslova et al., 2019). Here, the inventor describes a crystal structure of a human derived neutralizing antibody in complex with a GII.4 strain of NoV. The crystal structure of Nano-85, an alpaca derived nanobody, which is much smaller in molecular size when compared to a Fab, in complex with the GII.4 P domain was also recently reported (Koromyslova et al., 2015). Nano-85 binds toward the proximal end of the P domain dimer, which in the context of the capsid would be closer to the S and P domain interface. The epitope recognized by this nanobody which consists of W520, N522, and T526 is entirely distinct from that recognized by NORO-320. Based on negative-stain images of VLPs in complex with this nanobody it is suggested that the binding disrupts particle assembly.

It is indeed intriguing that even though NORO-320 does not directly bind in close proximity to the receptor binding domain it still inhibits GII.4 VLPs from binding to glycans in vitro and inhibits viral replication of GII.4 Sydney 2012 virus (Alvarado et al., 2018). The blockade studies using IgG and Fab recombinant variants, as well as the originally isolated antibody that was a dimeric IgA molecule, show that the molecular size of the mAb influenced the potency of neutralization (FIG. 15). Blocking potential increased with the increase in molecular size of the NORO-320 variant tested. Therefore, blockade potential is a result of NORO-320 sterically blocking GII.4 VLPs from binding to glycans in vitro. Modeling of the Fab binding to the P domain in the context of the capsid using the only available X-ray structure of the GI.1 indicates that constant domain of the Fab clashes with the neighboring VP1 subunits potentially affecting the particle assembly. But the inventor's observation that Fab alone does not block the HBGA binding suggest that Fab binding does not affect capsid structure, and it is possible that the orientation of the constant domain in the context of the capsid is slightly altered to avoid clashes. However, the additional Fc region projecting out from the Fab, as in the context of IgA or IgG, may sterically hinder the glycan binding sites in the neighboring VP1 subunits. Thus, this study potentially identifies a novel mechanism of neutralization for GII.4 Sydney 2012 NoV. The amino acids critical for the interaction between the GII.4 P domain and NORO-320 also are conserved among GII.3, GII.6 and GII.17 strains, which suggests that they are neutralized similarly. It is also possible the bound NORO-320 IgG or the IgA affecting the particle integrity leading to disassembly is an additional factor contributing to neutralization.

These results suggest that even though there is a high degree of sequence and antigenic diversity in the capsid protein VP1 among circulating strains of human NoVs, common protective antigenic sites exist among these strains. Recognition of the P2 subdomain and the most conserved S domain of VP1 by human mAbs could be the molecular basis for broad cross-reactive neutralization. The novel neutralizing NoV epitope identified also informs us about a critical antigenic GII site that could later be used in the reformulation of broadly protective NoV vaccine candidates. These human mAbs also could be directly used as a prophylactic, a therapeutic, or a reagent for diagnosis.

### Example 7—Materials and Methods

Generation of virus-like particles. Virus-like particles (VLPs) based on HuNoV strains GI.1 (M87661), GI.2 (AF435807), GI.3 (AF439267), GII.3 (TCH02-104), GII.4 (AFV08795.1), GII.6 (AF414410), GII.13 (JN899242) and GII.17 (AB983218) were expressed recombinantly and purified as previously described (Jiang et al., 1992). The inventor used a baculovirus recombinant protein expression system for VLP production. The inventor cloned the VP1 and VP2, major and minor, protein capsid sequences from each strain into the transfer vector pVL1392 (Epoch Life Science, Inc). Sf9 insect cells were co-transfected with a transfer vector corresponding to a specific strain and with a bacmid vector. Recombinant baculovirus was isolated and expanded. VLPs were purified from cell culture supernatants using a sucrose and cesium chloride gradient. VLP formation was verified using electron microscopy.

Reactivity to VLPs by ELISA. An ELISA was used to testing binding of human mAbs to VLPs, as was previously described (Sapparapu et al., 2016). Each VLP was coated individually at 1 μg/mL on 384-well microtiter plates at 4° C. overnight. Plates then were blocked for one hr at room temperature using 5% nonfat dry milk in PBS with 0.05% Tween-20. For screening and EC₅₀ analysis, antibody reactivity to VLPs was detected using horseradish peroxidase (HRP) tagged anti-κ or -λ chain secondary antibodies (Southern Biotech). 1-Step™ Ultra-TMB Substrate Solution (Pierce Thermo Fisher) was used to detect HRP activity.

Human subjects. The Vanderbilt University Medical Center Institutional Review Board approved the protocol used in this study in which six adult individuals participated. All participants provided written informed consent before blood samples were obtained. The subjects had a previous history of acute gastroenteritis but were otherwise healthy.

Human hybridoma generation. Human hybridomas secreting human mAbs were generated as previously described (Alvarado et al., 2018). Briefly, PBMCs were isolated from human subject blood samples using Ficoll-Histopaque and density gradient centrifugation and then cryopreserved. Later, cells were thawed, transformed using Epstein-Barr virus, CpG10103, cyclosporine A and a Chk2 inhibitor and plated in a 384-well plate. Transformed cells were incubated at 37° C. for 7 days, and then expanded into 96-well plates containing irradiated human PBMCs. Four days later, cell supernatants were screened by indirect ELISA for the presence of anti-norovirus VLP cross-reactive mAbs. B cells secreting cross-reactive mAbs were electrofused to HMMA2.5 myeloma cells and plated in medium containing hypoxanthine, aminopterin, thymidine and ouabain. Hybridoma cell lines were incubated at 37° C. for 14 days, and then supernatants were screened by indirect ELISA for production of cross-reactive mAbs. Cell lines expressing cross-reactive mAbs then were cloned biologically using single-cell fluorescence activated cell sorting.

Purification of cross-reactive mAbs. After cloning, hybridoma cell lines producing cross-reactive mAbs were expanded gradually from 48-well plates to 12-well plates, T-25, T-75 and eventually to four T-225 flasks for each cell line. Supernatant from each cell line also was screened by ELISA to determine the corresponding light chain for each clone. Following 4-weeks of incubation at 37° C., supernatant from the four T-225 flasks was harvested and filtered through a 0.4-μm filter. The supernatant was filtered using column chromatography, specifically HiTrap KappaSelect and Lambda FabSelect affinity resins (GE Healthcare Life Sciences). To obtain varying forms of NORO-320, the inventor expressed the heavy and kappa light chain variable domains using Fab or IgG protein recombinant expression vectors. cDNAs encoding the corresponding heavy and light chains were transfected using ExpiCHO™ (Chinese hamster ovary) cells (Thermo Fisher Scientific).

VLP-carbohydrate mAb blocking assay. To test the ability of each mAb to inhibit the interaction between the selected VLPs and glycans in vitro, the inventor used a blocking assay. As previously described, he coated microtiter plates with 10 μg/mL of pig gastric mucin Type III (Sigma) for 4 hr at room temperature. Porcine gastric mucin (PGM) purified from porcine stomach mucosa contains both H and Lewis antigens, α-1,2- fucose and α-1,4-fucose (Lindesmith et al., 2012) (Lindesmith et al., 2008) (Tian, P. et al., 2010). Plates then were blocked overnight at 4° C. in 5% nonfat dry milk. VLPs at 0.5 μg/mL were pretreated with serially diluted concentrations of each mAb for 1 hr at room temperature. The optimal concentrations of mAbs were normalized before testing blocking ability and tested at concentrations beginning at 1,000 nM and then diluted serially. VLP-mAb complexes were added to the PGM-coated and blocked microtiter plates. After 1 hr of incubation, the plates were washed 3 times with PBST and the same was done in between each step. Bound VLPs were tested using murine serum containing anti-GI.3, GII.4, GII.6 or GII.17 polyclonal antibodies, followed by an HRP conjugated goat anti-mouse IgG human adsorbed antibody. Optical density was measured at 450 nm using a Synergy HT Microplate Reader (BioTek). Blocking studies also were repeated three times.

Expression and purification of protruding and shell domain for selected HuNoV strains to be used in Ab binding studies. In order to map the epitope of cross-reactive mAbs, the inventor first recombinantly expressed P1 and P2 domain sequences or shell domain of GI.3 (AF439267), GII.4 (AFV08795.1), GII.6 (AF414410), GII.13 or GII.17 (AB983218). P-domain sequences were cloned into the pGEX-4T-1 expression vector with a GST tag and thrombin cleavage site. The P-domain then was expressed in Escherichia coli BL-21 cells and purified using a Glutathione Sepharose Fast Flow Column (GE Healthcare) and column chromatography. The S-domain sequences were cloned into pVL1392 and co-transfected with a bacmid vector into Sf9 insect cells. Recombinant baculovirus particles then were harvested and used to inoculate Sf9 cells. S-domain particles were then purified from the inoculated Sf9 cell culture supernatant using a sucrose and a cesium chloride cushion gradient.

Expression, purification and crystallization of GII.4 P-domain and NORO-320 Fab. The sequence for the GII.4 protruding domain was cloned into the expression vector pMal-C2E (New England BioLabs). The expression vector includes a N-terminal His₆-maltose binding protein (MBP) tag and a tobacco etch virus (TEV) protease cleavage site between the MBP and P-domain sequence. The P-domain was expressed in E. coli BL21(DE3) and purified using an AffiPure Ni-NTA agarose bead column (GenDepot). The His-MBP tag was then removed using TEV protease and separated from the P-domain by purifying it once again using His-Trap (GE Healthcare), MBPTrap (GE Healthcare) affinity columns and size exclusion chromatography. Finally, the purified P-domain was concentrated and stored in 20 mM Tris-HCl buffer (pH 7.2) containing 150 mM NaCl, and 2.5 mM MgCl₂.

The nucleotide sequences of the variable domain of mAb NORO-320 was optimized for mammalian expression and synthesized (GenScript) for expression and purification of recombinant Fab. The heavy chain fragment was cloned into a vector for expression of recombinant human Fabs (McLean et al., 2000). The light chain was cloned into a vector for κ light chain. Each vector was transformed independently into E. coli cells, and DNA then was purified. Both the heavy and light chain encoding vectors were transfected into CHO cells using an ExpiCHO™ expression system. Cell supernatant was collected, centrifuged and filtered using a 0.45 μm filter. NORO-320 Fab was purified by affinity chromatography using a KappaSelect (GE Healthcare).

Purified GII.4 P-domain and NORO-320 Fab were combined in a 1:1.5 molar ration and incubated for 1 hr at 4° C. The mixture was passed through an S200pg 16/60 gel filtration column, and the peak corresponding to the complex was collected. The size of the complex and presence of both proteins was validated on an SDS-PAGE gel. The peak fractions then were pooled and concentrated to 10 mg/mL for crystallization trials. Crystallization screening using hanging-drop vapor diffusion method at 20° C. was set up using a Mosquito nanoliter handling system (TTP LabTech) with commercially available crystal screens, and crystals were visualized by using a Rock Imager (Formulatrix). The GII.4 P-domain-NORO-320 Fab complex crystallized in a buffer containing 0.1 M BIS-TRIS prop 8.5 pH, 0.2 M KSCN, 20% w/v PEG 3350. Crystals diffracted to 2.25 Å resolution.

Diffraction, data collection, and structure determination. Diffraction data were collected on beamline 5.0.1 at Advanced Light Source (Berkeley, Calif.). Diffraction data were processed using HKL2000 (Otwinowski & Minor, 1997). The previously published GII.4 (strain TCH05) P-domain structure (PDB ID 3SJP) and the neutralizing Fab 512 (PDB ID 5KW9) were used as the search models by molecular replacement using program PHASER (McCoy et al., 2007). Iterative cycles of refinement and further model building were carried out using PHENIX (Adams et al., 2002) and COOT programs (Emsley & Cowtan, 2004). During the course of the refinement, and following the final refinement, the stereochemistry of the structures was checked using MolProbity (Chen, V. B. et al., 2010). Data refinement and statistics are given in Table Y. The interactions between P-domain and the Fab for NORO-320 were analyzed using LigPlot+ v.2.1 (Laskowski & Swindells, 2011). Figures were prepared using Chimera (Pettersen et al., 2004). Sequence alignment and sequence comparisons the inventor analyzed using EMBL-EBI multiple sequence alignment software (Madeira, F. et al., 2019).

Dynamic light scattering. The hydrodynamic diameters of treated or untreated HuNoV VLPs were measured using dynamic light scattering (DLS) on a ZetaSizer Nano instrument (Malvern Instruments, UK). Samples were diluted to a final concentration of 330 nM for each component in phosphate-buffered saline pH 6 and a 3,300 nM concentration of NORO-320 Fab was prepared for condition labeled VLP:NORO-320 Fab (1:10). Three×12 measurement runs were performed with standard settings (Refractive Index 1.335, viscosity 0.9, temperature 25° C.) for each time point. The average result was created with ZetaSizer software.

Detection of bis-ANS binding by fluorescence spectroscopy. Purified VLP (30 μg/mL, 0.5 μM concentration of the VP1) or 0.5 μM purified antibody (NORO-320 Fab, IgA, or 22D2 control) diluted in PBS buffer pH 6.0 was incubated at 25° C. or 37° C. to allow for temperature equilibration. To detect bis-(8-anilinonaphthalene-1-sulfonate) (bis-ANS) binding to native VLP and antibody, bis-ANS was added to the sample to a final concentration of 3 μM. Bis-ANS was excited at 395 nm and emission was collected at 495 nm at 30 second intervals for 15 minutes on a Flexstation 3 (Molecular Devices, USA). To investigate the binding of bis-ANS to preincubated VLP and antibody, GII.4 VLP and antibody were mixed and incubated for 10 minutes at 25° C. or 37° C. Bis-ANS then was added to sample, and samples were immediately transferred to a spectrofluorometer for reading as detailed above.

Virus neutralization assay. Human jejunal intestinal enteroids (J4^{Fut2} HIEs) were plated and differentiated as cell culture monolayers in collagen IV-coated 96-well plates in commercial Intesticult human organoid growth medium (INT; Stem Cell Technologies), as previously described (Atmar et al., 2020) (Haga et al., 2020)(Ettayebi et al., 2021). Prior to infection, 5-fold serial dilutions of NORO-320 IgA, NORO-320 Fab, or a dengue virus-specific control antibody were prepared in CMGF(−) medium supplemented with 500 μM glycochenodeoxycholic acid (GCDCA; Sigma, G0759), and each dilution or the medium control was mixed in equal volume with 100 TCID₅₀ of GII.4 (GII.P31/GII.4-Sydney/TCH12-580). NORO-320 Fab antibody was also tested to neutralize GII.17 (GII.P13/GII.17/1295-44). The antibody fragment:virus mixtures were preincubated for 1 hr at 37° C. prior to inoculation onto triplicate wells of the differentiated J4^{Fut2} HIE monolayers and incubated for an additional 1 hr at 37° C. After 1 hr post-infection (hpi), monolayers were washed twice with CMGF(−) medium and incubated with differentiation INT medium supplemented with 500 μM GCDCA. After 1 hpi (immediately after wash) and 24 hpi, cells and medium were collected, and RNA was extracted using KingFisher Flex Purification System and MagMax Viral RNA Isolation kit. RNA extracted at 1 hpi was used to determine a baseline value for the amount of input virus that remained associated with cells after washing the inoculated cultures. Virus replication was assessed by quantifying virus genome equivalent levels (GEs) from samples extracted at 24 hpi in comparison to the 1 hpi time point. Percent reduction in GEs relative to medium (100%) then was determined. Reverse-transcription quantitative polymerase chain reaction (RT-qPCR) was performed as described previously (Ettayebi, K. et al., 2016).

### Example 8—Results

Isolation of broadly binding anti-HuNoV human mAbs. To isolate cross-reactive HuNoV human mAbs, the inventor used EBV and additional B cell stimuli to transform memory B cells in PBMCs obtained from patients who were overall healthy but with a previous history of acute gastroenteritis, as previously described (Sapparapu, G. et al., 2016). A week later, transformed PBMCs supernatants were tested by ELISA to screen for the expression of mAbs that bound to more than one representative strain HuNoV VLP. The VLPs used to screen were HuNoV GI.1, GI.2, GI.3, GII.3, GII.4, GII.6, GII.13 or GII.17. Each VLP was coated individually and blocked on a microtiter plate before the screening. The bound antibodies were detected using alkaline phosphatase-conjugated goat anti-human κ or λ chain secondary antibodies to capture binding activity by any antibody isotype. Wells that contained transformed B cells expressing mAbs that recognized more than one VLP then were expanded. B cells secreting anti-NoV mAbs were rescued by hybridoma formation. Binding reactivity to pandemic GII.4 Sydney 2012 was previously characterized for 5 of the mAbs included in the panel (Alvarado et al., 2018). The heavy and light variable gene regions were sequenced for all 12 mAbs and the V, D, J, and other variable gene sequence features were analyzed (Brochet et al., 2008) (data not shown). Each of the mAbs had unique variable gene sequences, suggesting that cross-reactivity was not limited to one antibody clonotype.

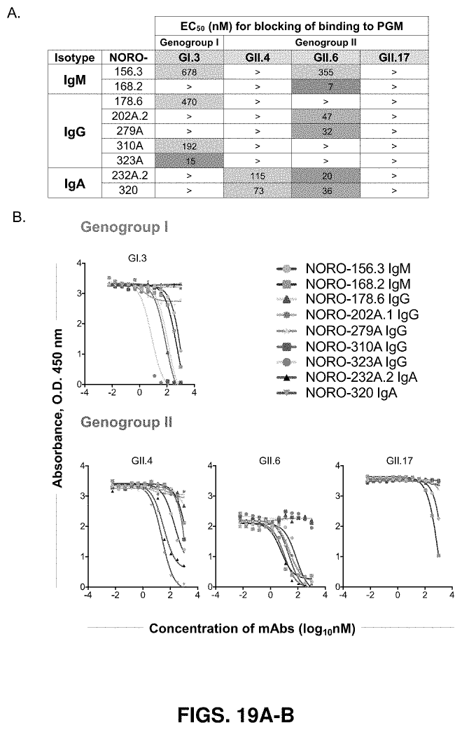

Binding and blocking activity of cross-reactive mAbs to HuNoV GI and GII VLPs. To assess the binding reactivity and blocking the function of the 12 mAbs, the inventor used indirect ELISA and a VLP blocking assay. The concentration of each mAb was normalized first for the number of antigen-binding sites. The inventor then tested binding starting at a concentration of 500 nM, followed by 11 serial dilutions. He used these data to determine the EC50 value of each mAb when binding to HuNoV GI.1, GI.2, GI.3, GII.3, GII.4, GII.6, GII.13 or GII.17 VLPs (FIGS. 18A-B). Binding reactivity revealed 3 distinct binding patterns. NORO-168.2, -156.3, and -170.5, all IgMs, each exhibited wide breadth by binding to all VLPs tested. Both IgAs, NORO-232A.2 and -320 as well as two IgGs, NORO-167.3 and -202A.1 exhibited specificity of binding only for GII variants. The remaining mAbs, NORO-155.5, -178.6, -279A, -310A, and -323A reacted with at least one GI and one GII strain. These binding patterns from natural infection also have been reported recently to follow similar trends in HuNoV vaccination trials (Lindesmith et al., 2019). These studies suggest that typical adult human B cell responses to HuNoV antigens include clonotypes encoding both broadly reactive non-neutralizing antibodies and more narrowly reactive neutralizing antibodies.

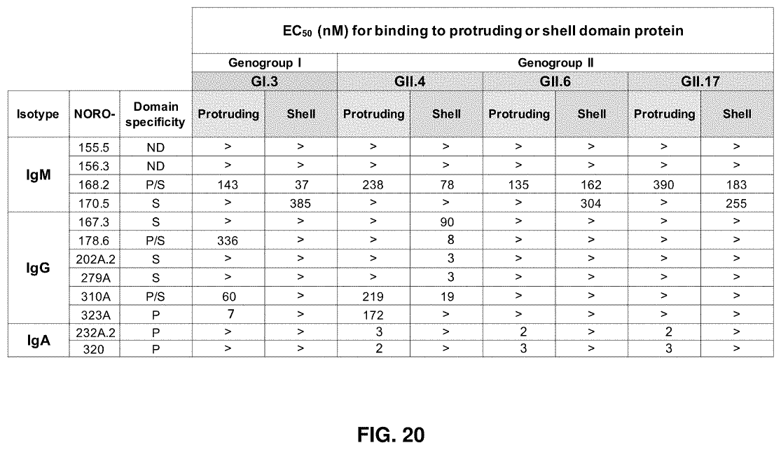

To determine if any of the isolated cross-reactive mAbs had functional activity, the inventor used a surrogate system to analyze neutralization using porcine gastric mucin (PGM)(Lindesmith et al., 2012) as described in the Example 7 (FIGS. 19A-B). He first tested if GI.1, GI.2, GI.3, GII.3, GII.4, GII.6, GII.13, or GII.17 VLPs could bind to the glycans present in PGM and found that GI.3, GII.4, GII.6 and GII.17 VLPs bound to PGM. Therefore, the inventor tested inhibition of binding of GI.3, GII.4, GII.6, and GII.17 VLPs to PGM. NORO-155.5 and -170.5, both IgMs, and -167.3, and IgG, did not inhibit any of the VLPs tested from binding to PGM in vitro. 9 of the 12 mAbs blocked at least 1 of the 4 VLPs tested. None of the 8 mAbs with binding reactivity to GII.17 VLPs had any strong blocking activity with GII.17; two clones exhibited some activity, but the EC₅₀ values were estimated to be >1,000 nm. NORO-320, an IgA that bound broadly across selected GII strains, also blocked GII.4 and GII.6 VLPs from binding to PGM but not GII.17. The absence of GII.17 blocking may stem from a difference in glycans in the pig gastric mucin the inventor used compared to the native human cellular glycans to which GII.17 viruses bind. Another possibility is that the HBGA binding site remains available even when NORO-320 IgA mediates particle aggregation or disassembly, as suggested by results using dynamic light scattering (see below).

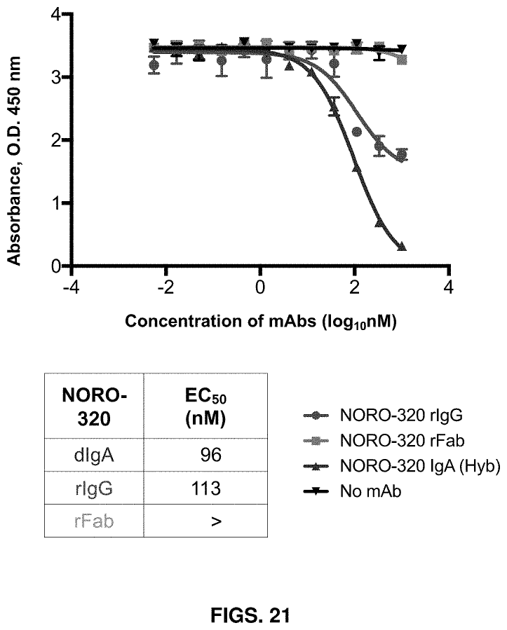

Binding to GI.3, GII.4, GII.6, or GII.17 variant protruding (P) vs shell (S) domains. The major capsid protein, VP1, which forms the icosahedral capsid, is divided into the protruding (P) and shell (S) domains (Prasad et al., 1996). To map where the cross-reactive mAbs bind, the inventor first expressed and purified recombinant proteins for GI.3, GII.4, GII.6, and GII.17 HuNoV strains using P-domains expressed in Escherichia coli BL-21 cells and S-domains expressed in Sf9 insect cells. Antibody binding to S, P, and VLPs, containing both S and P-domains, was tested and compared. P and S-domain recombinant proteins were coated at equal concentrations of 2 μg/mL on microtiter plates and blocked with 5% nonfat dry milk in 1×DPBS with 0.05% Tween-20. Before adding the mAbs to plates, each mAb was normalized according to the number of antigen binding sites. The inventor tested binding starting at a concentration of 500 nM followed by 11 serial dilutions of each mAb to obtain the half-maximal binding concentrations. Both IgAs, NORO-232A.2 and -320, appeared to bind specifically to the GII.4, GII.6 and GII.17 P-domains (FIG. 20). The inventor observed wide ranges of EC₅₀ values (2 to 390 nM for P-domains and nM μg/mL for S-domains). NORO-320 had some of the lowest EC₅₀ values of all the mAbs screened, with EC₅₀ values of 2 or 3 nM for each of the P-domains tested. Similarly, previously isolated cross-reactive murine mAbs also have been mapped to the NoV P-domain (Parker et al., 2005). NORO-168.2 bound to both the P and S-domains of GI.3, GII.4, GII.6 and GII.17, but in all instances had a lower EC₅₀ value when bound to the S-domain. Some mAbs like NORO-155.5 and -156.3 did not bind to any of the P or S-domains tested. Loss of binding may suggest that these mAbs require both the S and P-domain to be present for Ab binding.

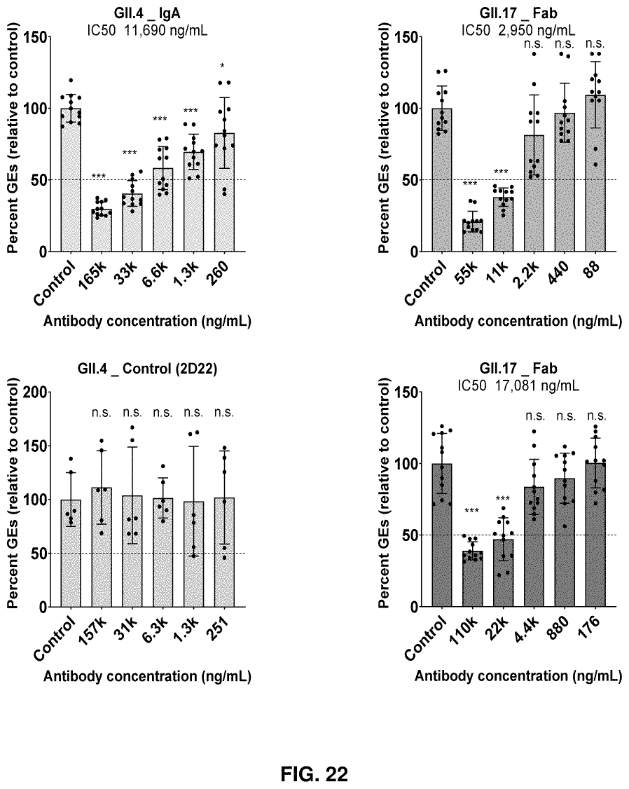

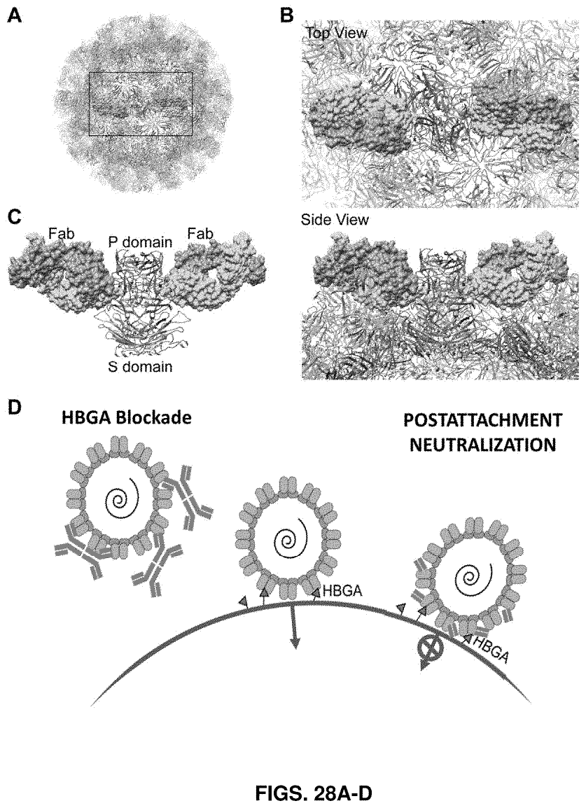

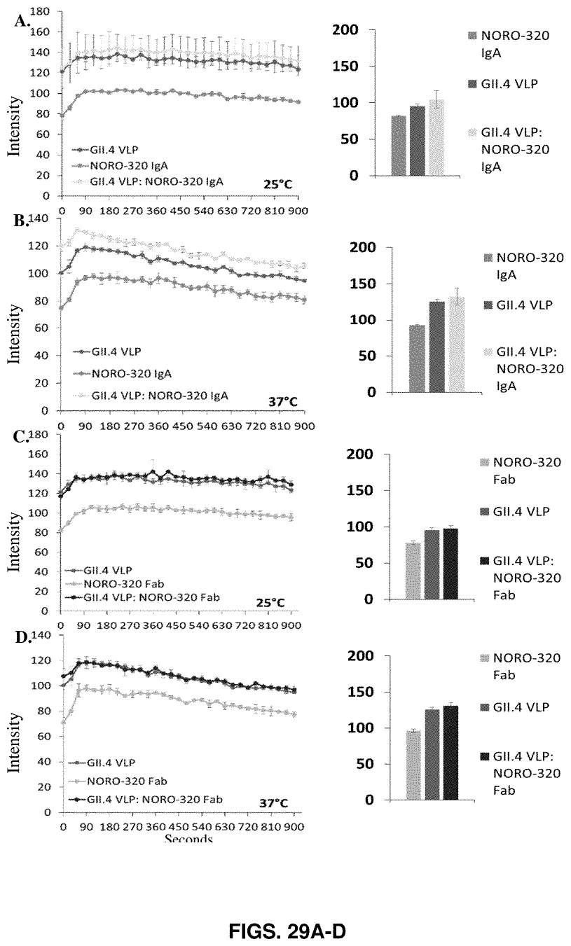

HBGA-blocking and neutralization of HuNoV infection by NORO-320 mAb and Fab. In previous studies, the inventor determined that NORO-320 inhibits GII.4 Sydney 2012 virus replication when using a human intestinal enteroid culture (Alvarado et al., 2018). To determine if NORO-320 neutralizes GII.4 because it sterically hinders HuNoV binding to glycans, he tested if blocking activity was influenced by the molecular weight or size of NORO-320, using recombinant Fab, IgG, or IgA isotypes of NORO-320. To verify the molecular weight of the original hybridoma-expressed IgA and each of the recombinantly expressed mAbs, 4 μg of each mAb, along with a set of control mAbs of known molecular weight, were resolved on an SDS-PAGE gel under non-reducing conditions (FIG. 27). All the mAbs were of the expected apparent molecular weight, dIgA˜350 kDa, IgG˜150 kDa, and Fab˜50 kDa. The inventor then tested NORO-320 IgA, recombinant IgG, recombinant Fab, and an irrelevant mAb as a control for their ability to inhibit GII.4 Sydney 2012 VLPs from binding to PGM in vitro. As hypothesized, blocking activity varied by the form of the antibody. The large NORO-320 IgA had the lowest EC₅₀ value followed by NORO-320 recombinant IgG (FIG. 21). Recombinant NORO-320 Fab did not block GII.4 Sydney 2012 VLPs from binding to PGM at concentrations as high as 1,000 nM. This finding indicates that the dimeric NORO-320 likely neutralizes GII strains broadly because of the capacity of this large molecule to mediate steric hindrance to receptor binding by cross-linking and aggregating viral particles (FIGS. 28A-D).

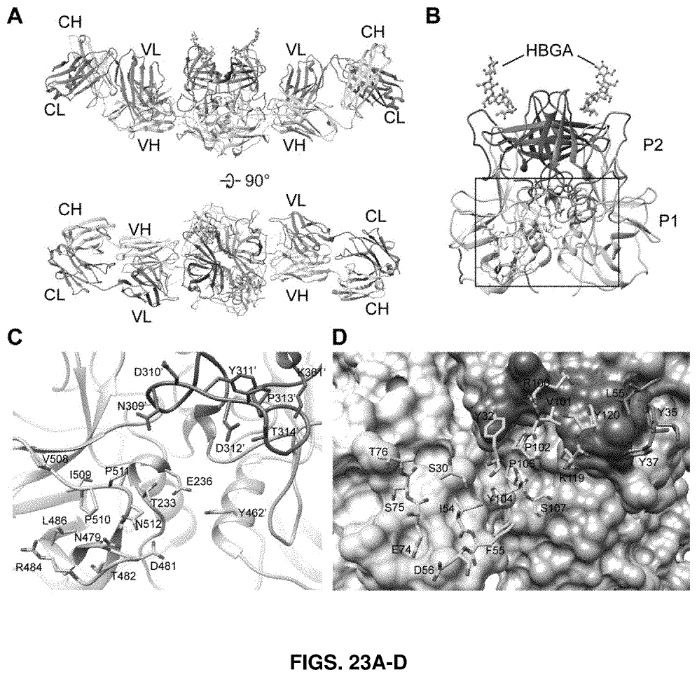

Furthermore, to examine if the NORO-320 Fab also lacks the ability to neutralize the infectious virus, the inventor performed neutralization assays using the enteroid culture system (Ettayebi, K. et al., 2016). Remarkably, he observed that NORO-320 Fab mediated neutralization of viral replication for both GII.4 and GII.17 HuNoVs (FIG. 22). While NORO-320 IgA neutralizes GII.4 with a high ICso of 11,690 ng/mL, the NORO-320 Fab exhibits an ICso of 2,950 ng/mL.

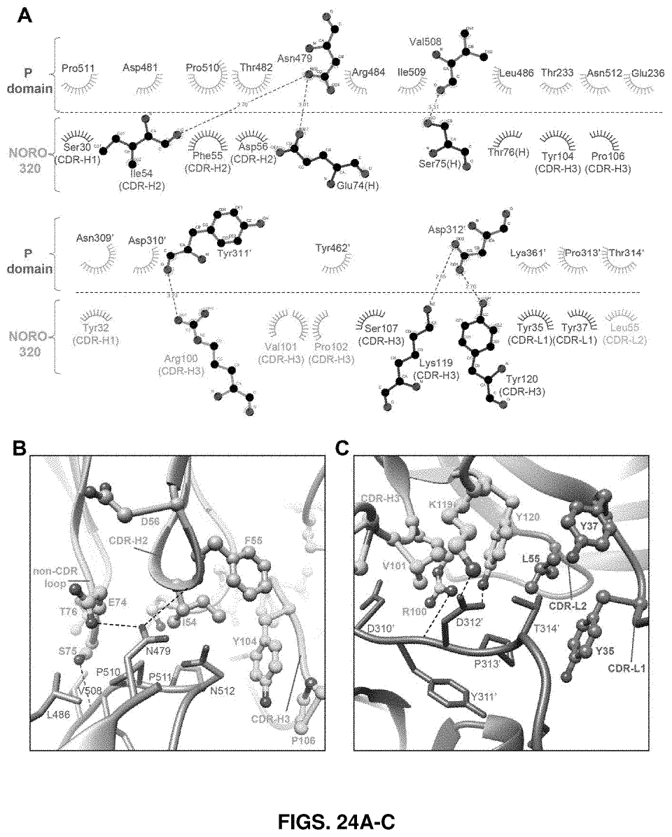

Crystal structure of NORO-320 Fab in complex with GII.4 P-domain. To understand how NORO-320 binds so broadly and neutralizes diverse GII HuNoV strains without blocking glycan binding, the inventor determined the crystal structure of the NORO-320 Fab in complex with the GII.4 P-domain at a resolution of 2.3 Å (FIGS. 23A-B). According to the structure, NORO-320 Fab binds perpendicular to the 2-fold axis of the P-domain dimer near a region close to the shell domain and significantly distant from the HBGA binding site to inhibit GII.4 VLP-carbohydrate binding. The superimposition of the structures of GII.4 P-domain in complex with NORO-320 and in complex with HBGA revealed that the P-domain dimer structure remains invariant, with an r.m.s.d. of ˜1.1 Å for the matching Ca atoms. These structural observations indicate that the Fab form of NORO-320 cannot affect glycan-binding either directly or allosterically. These observations are consistent with the inventor's findings from the HBGA blocking assays showing that NORO-320 Fab does not inhibit glycan binding.

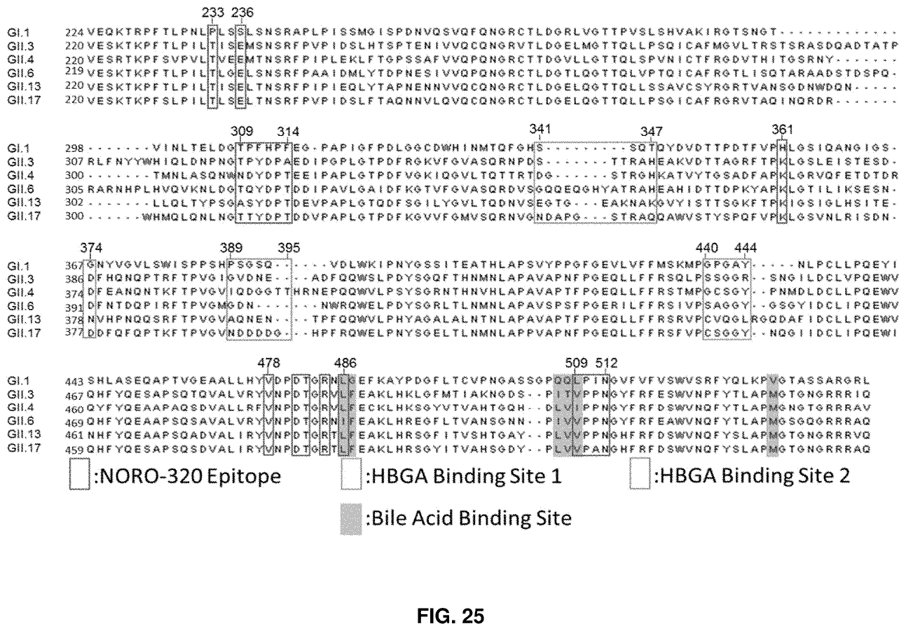

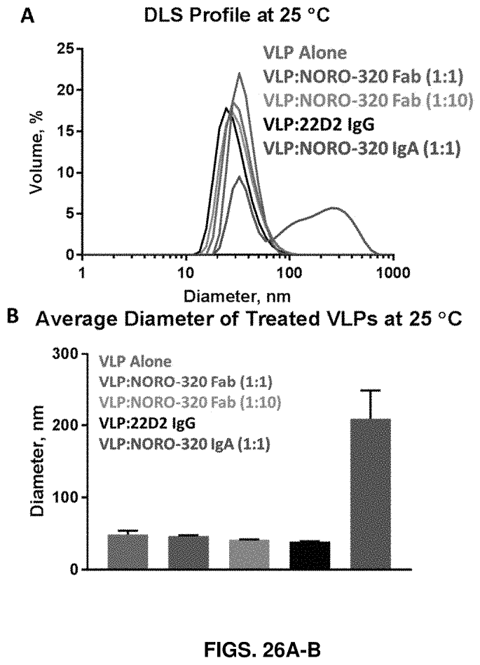

Molecular details of recognition of GII.4 P-domain by NORO-320. The crystal structure of NORO-320 Fab-GII.4 P-domain complex shows that the antibody makes extensive interactions with the P-domain. The Fab binding site on the P-domain is formed by residues from the P1 subdomain of one subunit and the P2′ subdomain of its dimeric partner (FIGS. 23C-D). The paratope in NORO-320 includes residues from the CDRs of both light and heavy chains. The Fab binding is stabilized by both hydrogen bond and hydrophobic interactions (FIG. 24A). For instance, the sidechain of N479 in the P1 subdomain hydrogen bonds with 154 from CDRH2 and E74 of a non-CDR loop, whereas residues L486, V508, P510, P511, and N512 are involved in hydrophobic interactions with residues from CDRH2 and CDRH3 (FIG. 24B). Residue D312 of the P2′ subdomain interacts with K119 and Y120 of CDRH3, involving both direct hydrogen bond and hydrophobic interactions (FIG. 24C). In addition to CDRHs, three light chain residues Y35 and Y37 of CDRL1 and L55 of CDRL2 form hydrophobic interactions with P313′ and T314′ of the P1′ subdomain. To understand how NORO-320 can bind to VLPs of various GII strains, the inventor aligned the P-domain amino acid sequences of GI.1, GII.3, GII.4, GII.6, GII.13, and GII.17 (FIG. 25). Sequence alignment revealed 78 to 89% conservation at these sites among the GII strains compared to 54 to 59% conservation for the entire P-domain sequence. In contrast, the epitope sequence in the GI genogroup shows significant changes in this region accounting for only 44% sequence similarity. The high level of sequence conservation within the epitope of GII strains provides insight into why NORO-320 binds broadly among GII.3, GII.4, GII.6, GII.13, and GII.17 strains.

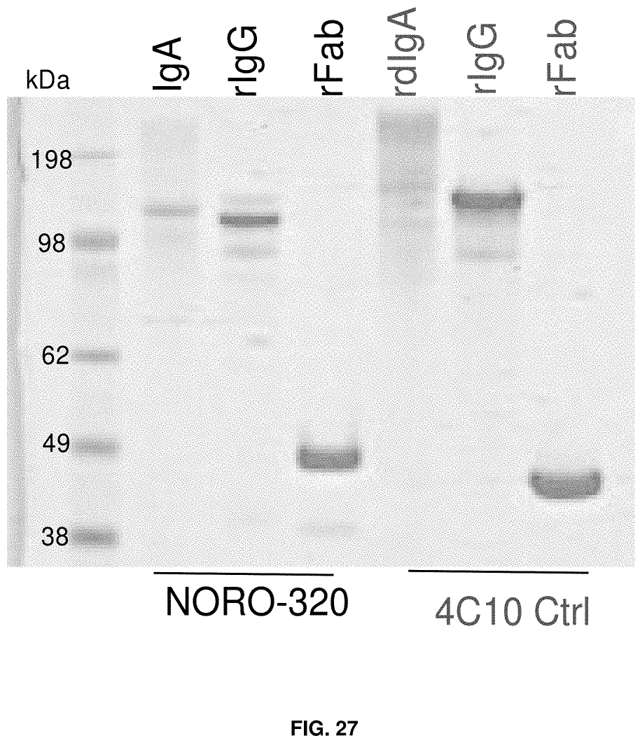

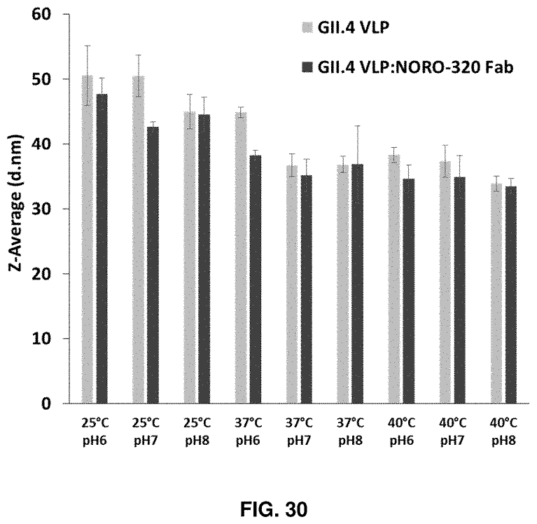

Dynamic light scattering analysis of GII.4 particle integrity by NORO-320. To investigate the effects of binding of NORO-320 mAb or Fab in the context of GII.4 capsid, the inventor carried out dynamic light scattering studies with GII.4 VLPs (FIGS. 26A-B). These studies showed that with GII.4 VLP and the IgA form of NORO-320, a large fraction of the sample has particles of 200 to 500 nm in diameter. This 200 to 500 nm peak would correspond to clumping of approximately 4-12 intact viral particles, based on an average VLP diameter of 40 nm. In contrast, when GII.4 VLPs were treated with NORO-320 Fab at a 1:1 or 1:10 ratio of VP1:Fab, the inventor did not observe an alteration in particle diameter (FIG. 26B). GII.4 VLPs were also treated with NORO-320 Fab at pH 6, pH 7, or pH 8 and incubated at 25° C., 37° C., or 40° C. for 30 minutes (FIG. 30). He did not observe an increased susceptibility to particle aggregation or disassemble based on pH or temperature.

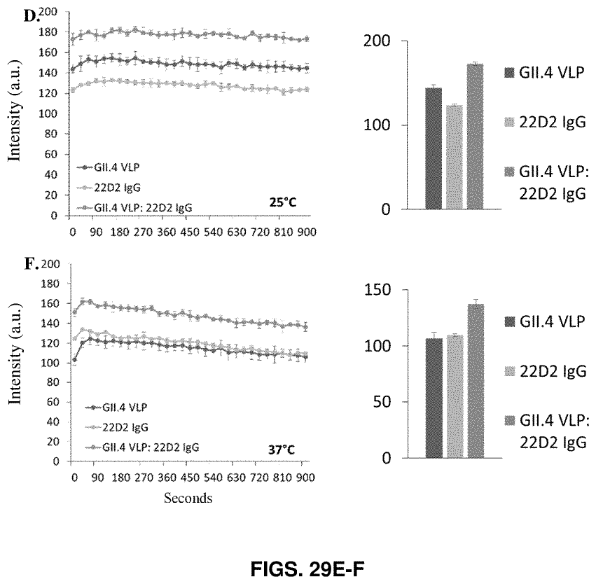

Bis-ANS assay to probe local conformational changes. To further investigate the possibility that binding of NORO-320 Fab could induce local conformational changes in the GII.4 VLP, the inventor used a bis-ANS fluorescence assay. This approach has been used previously to detect possible local conformational changes in feline calicivirus capsid protein upon incubation with the soluble cellular receptor fJAM-A (Ossiboff et al., 2010). Upon incubation of GII.4 VLP with NORO-320 IgA or Fab, the inventor did not detect a significant increase in bis-ANS fluorescence at 25° C. or 37° C. (FIGS. 29A-F). These studies, consistent with the results from the DLS experiments, suggest that binding of NORO-320 Fab does not cause any significant conformational changes in the VLP.

TABLE Y
Data processing and refinement statistics for GII.4
P-domain-NORO-320 Fab complex

| Data Collection | |
|---|---|
| Beamline | ALS Beamline 5.0.1 |
| Wavelength, Å | 0.97741 |
| Space group | P21 21 2 |
| Cell dimensions, Å | 119.25, 186.27, 73.44 |
| α, β, γ, ° | 90, 90, 90 |
| Resolution, Å | 50-2.25 (2.29-2.25)^{a} |
| Total Reflections | 1716311 |
| Unique Reflections | 78053 (3854)^{a} |
| Redundancy | 6.5 (6.2)^{a} |
| Completeness (%) | 99.82 |
| <I/sigma> | 15.6875 (2.375)^{a} |
| Rₘₑₐₛ ^{b} | 0.129 (0.846)^{a} |
| Rₚᵢₘ ^{b} | 0.050 (0.340)^{a} |
| Refinement Statistics | |
| Resolution, Å | 50-2.25 (2.29-2.25)^{a} |
| Reflections (work) | 73965 |
| Reflections (Test) | 3926 |
| R_{work} ^{C}/R_{free} ^{d} (%) | 18.08/22.55 |
| No. of Atoms | |
| Protein | |
| P-domain Dimer | 4798 |
| Noro-320 Fab | 6674 |
| Water | 1059 |
| Average B Value (Å²) | |
| P-domain Dimer | 34.2505 |
| NORO-320 Fab | 31.67 |
| Water | 36.085 |
| RMSD from Ideal Geometry | |
| Bond length (Å) | 0.003 |
| Bond angle (°) | 0.614 |
| Ramachandran Statistics ^{e} | |
| Favored | 98.38% |
| Outliers | 0.20% |

^{a}Numbers in parentheses refer to the highest resolution shell
^{b} Rₘₑₐₛ = Σhkl {N(hkl)/[N(hkl) − 1]}^{1/2} X Σᵢ|Iᵢ(hkl) − {I(hkl)}|/Σₕₖₗ ΣᵢIᵢ(hkl) and Rₚᵢₘ = Σₕₖₗ (l/(n − l))^{1/2} Σᵢ|Iₕₖₗ,ᵢ − |/ Σₕₖₗ Σi I_{hkl,i}, where Iₕₖₗ,i is the scaled intensity of the i^{th} measurement of reflection h, k, 1, is the average intensity for that reflection, and n is the redundancy.
^{C} R_{work} = Σₕₖₗ|Fo − Fc|/Σₕₖₗ|Fo|× 100, where Fo and Fc are the observed and calculated structure factors, respectively.
^{d} R_{free} was calculated as for R_{work}, but on a test set comprising 5% of the data excluded from refinement.
^{e} Calculated with MolProbity⁴¹.

### Example 9—Discussion

Isolation of naturally occurring broad-spectrum human mAbs to HuNoV holds great promise for the discovery of new candidate therapeutics, as well as identifying critical epitopes for the rational design of new structure-based broadly protective HuNoV vaccines. In the past, the genetic and antigenic diversity across circulating strains of HuNoV has made the generation of a broadly immunogenic vaccine extremely difficult. The primary goal of this study was to define the molecular and structural determinants of cross-reactivity and neutralization, using human mAbs to circulating strains of HuNoV. Previous studies have characterized the antigenic landscape of specific HuNoV strains, but with the rapid emergence of new genetically diverse strains, there is a need to map new immunogenic epitopes. This new information builds upon previous studies to help track the evolution of HuNoVs (Lindesmith et al., 2013). Identification of antigenic epitopes using human mAbs also will provide insight into the immunogenicity of HuNoVs. Of particular note, the binding patterns of clones in these panels of mAbs isolated from individuals following natural infection are consistent with recently reported data reported in human norovirus vaccination trials. In those studies, investigators identified one class of HuNoV circulating antibodies that exhibit extensive binding breath recognizing GI strains and GII strains but having no blocking activity and identified a second class containing antibodies that exhibit a more narrow range of reactivity but have blocking and neutralization activity (Lindesmith et al., 2019). These studies consistently show that human B cell responses to infection or vaccination with HuNoV antigens induce both broadly reactive non-neutralizing antibodies and strain-specific neutralizing antibodies.

Here, the inventor described the isolation of 12 anti-NoV cross-reactive human mAbs, 4 IgMs, 6 IgGs, and 2 IgAs, from subjects with a previous history of acute gastroenteritis. To determine the functional activity of the isolated mAbs, he used a previously validated surrogate blocking assay that measures the inhibition of VLPs from binding to glycans in vitro (Reeck et al., 2010). Not all of the strains tested bind to the same glycan, so he was only able to test the inhibition of binding for HuNoV VLPs GI.3, GII.4, GII.6, and GII.17. Of the 12 mAbs isolated, 9 mAbs blocked at least 1 of VLPs tested from binding to PGM with EC₅₀ values less than 1 μM. It should be noted that the use of a single VLP to represent a genotype is not comprehensive, as significant sequence variation exists within genotypes.

Prior to these studies, the inventor expected that cross-reactive and neutralizing human mAbs would bind predominantly to the P subdomain of the circulating strains, similar to the findings for cross-reactive murine Abs (Crawford et al., 2015). The P-domain has more surface exposure on a viral particle than the S-domain and should, therefore, be more accessible than the S-domain. Here, the inventor describes a crystal structure of a human-derived neutralizing antibody in complex with a GII.4 strain of HuNoV. Both previously characterized mAbs, the human IgA 5I2 and the mouse IgG 10E9, target regions adjacent or at the HBGA-binding site on the P2 subdomain thereby directly preventing the binding of glycans (Koromyslova et al., 2019)(Shanker et al., 2016). The crystal structure of Nano-85, an alpaca-derived nanobody, which is much smaller in molecular size when compared to a Fab, in complex with the GII.4 P-domain also was reported recently (Koromyslova & Hansman, 2015). Nano-85 binds toward the proximal end of the P-domain dimer, which in the context of the capsid would be closer to the S and P-domain interface. The epitope recognized by this nanobody which consists of W520, N522, and T526 is distinct from that recognized by NORO-320. Based on negative-stain images of VLPs in complex with this nanobody, it was suggested that the binding disrupts particle assembly. The crystal structure of the broadly reactive GII.4-blocking human antibody A1431 in complex with the GII.4 P-domain also has been reported recently¹³. Unlike NORO-320, mAb A1431 recognizes an epitope on the P-domain protomer within the P1 and P2 subdomain cleft, primarily recognizing residues Q402, W403, Q504, and D506. In comparison to this GII.4-specific antibody, NORO-320 recognizes a highly conserved epitope that allows it to recognize not only GII.4 variants, but additional GII genotypes, and it mediates HBGA blocking for GII.6 (FIG. 19A).

It is indeed intriguing that even though NORO-320 does not directly bind in close proximity to the HBGA binding site, it still inhibits GII.4 and GII.6 VLPs from binding to glycans in vitro and inhibits viral replication of GII.4 Sydney 2012 and GII.17 viruses (Alvarado et al., 2018). Blocking studies using IgG and Fab recombinant variants, as well as the originally isolated antibody that was a dimeric IgA molecule, show that the molecular size of the mAb influenced the degree of HBGA blocking (FIG. 21). Here, blocking potential increased with the increase in molecular size of the NORO-320 variant tested. Therefore, blocking appears to result from the action of NORO-320 to block GII.4 VLPs sterically from binding to glycans in vitro. The additional Fc region projecting out from the Fab, as in the context of IgA or IgG, may sterically hinder the glycan binding sites in the neighboring VP1 subunits.

Interestingly, however, when the inventor performed neutralization assays using the previously characterized enteroid culture system, he observed recombinant NORO-320 Fab exhibited similar levels of neutralization in comparison to full-length IgA and mediated neutralization of GII.17 (FIG. 22). Modeling of the Fab binding to the P-domain in the context of the capsid using the only available X-ray structure of the GI.1 indicates that the constant domain of the Fab likely clashes with the neighboring VP1 subunits. This conflict may affect particle disassembly, which may explain in part the mechanism by which NORO-320 Fab neutralizes GII.4 infection (FIGS. 28A-D. However, the DLS data presented here indicate that binding of NORO-320 Fab does not compromise particle integrity, because the diameter of the VLPs remains the same as that of the VLPs in the absence of the NORO-320 Fab (FIGS. 26A-B). In contrast, the inventor observes a significant increase in particle size from 40 nm to 210 nm with IgA, strongly suggestive of particle aggregation due to multivalent cross-linking, or aggregation of the sub-assemblies following particle disassembly. The results from the bis-ANS fluorescence assay also suggest that the binding of NORO-320 Fab does not cause any significant conformational changes in the VLP (FIGS. 29A-F). Based on these observations, the inventor suggests that NORO-320 IgA likely mediates neutralization principally by particle aggregation or disassembly of GII.4 particles. Since the Fab form of NORO-320 also mediates neutralization without causing aggregation, there must also be additional inhibitory features of this antibody that were not defined here that contribute to neutralization.

A recent study (Song, et al., 2020) has reported that GII.3 VLPs exhibit two different conformations of the P-domain dimer in which one conformation the P-domain is rotated by ˜70° and elevated above the shell domain compared to the other conformation. A similar rotated and elevated state also is observed in the case of murine norovirus capsid structure (Sherman, et al., 2019). While the mechanism and impact of this conformational change are not yet clear for HuNoV, for murine norovirus such a conformational change contributes to increased accessibility to its cellular receptor CD3001f and enhancement of infection efficiency (Graziano et al., 2020) (Nelson et al., 2018) (Katpally et al., 2008) (Katpally et al., 2010) (Hansman et al., 2012). The binding of NORO-320 Fab possibly could play a role in impeding such rotation and elevation between conformations, resulting in lower infection efficiency, although the inventor did not demonstrate this effect directly. Such an effect of this Fab could pertain to GII.3, GII.6, and GII.17 viruses, since the amino acids critical for the interaction between the GII.4 P-domain and NORO-320 are conserved those strains.

Taken together, these results presented here suggest that although there is a high degree of sequence and antigenic diversity in the capsid protein VP1 among circulating strains of HuNoVs, common protective antigenic sites exist among these genotypes. Recognition of the P1 subdomain and the more conserved S-domain of VP1 by human mAbs could be the molecular basis for broad cross-reactive neutralization. The novel neutralizing HuNoV epitope identified also informs us about a critical antigenic GII site that could later be used in the reformulation of broadly protective HuNoV vaccine candidates. These human mAbs also could be directly used as a prophylactic, a therapeutic, or a reagent for diagnosis.

TABLE 1
NUCLEOTIDE SEQUENCES FOR ANTIBODY VARIABLE REGIONS

| | | SEQ ID |
|---|---|---|
| Clone | Variable Sequence Region | NO: |
| NORO- | gaggttcacctggtggagtctgggggaggcctggtcaagcctggggggtccctgagactctcctgtgcagc | 1 |
| 246A | ctctggattcaccttcagtcgctatggcatgcactgggtccgccaggctccagggaaggggctggagtggg | |
| Heavy | tctcatccattgttattaggagtgattacaaatattatgcagactcagtgaagggccgattcaccatctccag | |
| | agacaacgccaagaattcactgtatctgcaaatgaacagcctcagagccgacgacacggccgtatattat | |
| | tgtgcgagagatctcgtggaaccgggtatcacaccagttggttgtgactactggggccggggaaccctggt | |
| | caccgtctcctca | |
| NORO- | tccaatgagctgacccaggcaccctcggtgtcagtgtccccaggacagacggccagaatcacctgctttgg | 2 |
| 246A | agatgcattggcaaatcaatattcttattggtaccagcggaagccaggccaggcccctgtcttggtgatata | |
| Light | taaagacagtgagaggccgtcagggatccctcagcgattctctgcctccaactcagggacaacagtcacg | |
| | ttgaccatcactggagtccaggcagaagacgaggctgactatttctgtcaatcggcagacagtactgggat | |
| | ctacaaggtgttcggcggtgggaccaagctgaccgtccta | |
| NORO- | caggtgcagctaaaacagtggggcgcagggctgttgaagtcctcggagaccctgtccctcacgtgcgcttt | 3 |
| 232 | caatggcaactccttcggtgctttctattggagttggatccggctgtccccagggaaggggctggagtggat | |
| Heavy | tggggaagtaaattatctaggaggtgccgactacaacccgtcgctcaagagtcgggtcaccatgtcggca | |
| | gacacgtccaagaggcagttctccctgagcctcaagtctgtcaccgccgcggacaccggtgtctatttttgt | |
| | gcgagaggtcggccccatgactactcgccggggagttattctcgccctcggcgttattacggtttggacgtc | |
| | tggggccaagggaccacggtcaccgtctcctca | |
| NORO- | gagattgtgttgacgcagtctccagccaccctgtctttgtctccaggggacagcgccaccctctcctgcagg | 4 |
| 232 | gccagtcaggctgttagcaccacctacttagcctggtaccagcagacacgtggccaggctcccagactcct | |
| Light | catccatggtacatacaccagggccattggtatcccagacaagttcagtggcactgggtctgggacagact | |
| | tcactctcaccatcagcggactggcgcctgaggattttgcagtgtattactgtcagcaatatagtagctcac | |
| | cgtacacctttggccaggggaccaagattgagatcaca | |
| NORO- | caggtgcaactggaagtgtctgggggaggcttggtcaagcctggagggtccctgagactctcctgtgcagc | 5 |
| 327A | ctctggattcaccttcactgactactacatgagttggatccgccaggctccagggaaggggctggagtggg | |
| Heavy | ttgcatacattagcggtataatgagttccacaaagtacgcagactctgtgaagggccgattcaccatctcca | |
| | gagacaacggcaagaactcagtgtatctgcaaatgaacagcctgacagccgaagacacggctgtctatta | |
| | ctgtgcgagagagagagtagaaccgccatctgctgtcactgacttctggggccagggaaccctggtcacc | |
| | gtctcctca | |
| NORO- | cagtctgtgctgacgcagtcgccctcagtgtctggggccccagggcagagggtcaccatctcctgcactgg | 6 |
| 327A | gaccagctccaacatcggggcaggttatgatgtacactggtatcagcagtttcctggaacagcccccaaac | |
| Light | tcctcatctctcataacaccaatcggccctcaggggtccctgaccgattctctggctccaagtctggcacctc | |
| | agcctccctggccatcactgggctccaggctgaggatgaggctgattattactgccagtcctttgacagcag | |
| | cctgcggggttccagggtgttcggcggagggaccaagctgaccgtccta | |

TABLE 2
PROTEIN SEQUENCES FOR ANTIBODY VARIABLE REGIONS

| | | SEQ ID |
|---|---|---|
| Clone | Variable Sequence | NO. |
| NORO- | QVQLKQWGAGLLKSSETLSLTCAFNGNSFGAFYWSWIRLSPGKGLEWIGEVNYLGG | 7 |
| 232 | ADYNPSLKSRVTMSADTSKRQFSLSLKSVTAADTGVYFCARGRPHDYSPGSYSRPRR | |
| Heavy | YYGLDVWGQGTTVTVSS | |
| NORO- | EIVLTQSPATLSLSPGDSATLSCRASQAVSTTYLAWYQQTRGQAPRLLIHGTYTRAIGI | 8 |
| 232 | PDKFSGTGSGTDFTLTISGLAPEDFAVYYCQQYSSSPYTFGQGTKIEIT | |
| Light | | |
| NORO- | EVHLVESGGGLVKPGGSLRLSCAASGFTFSRYGMHWVRQAPGKGLEWVSSIVIRSD | 9 |
| 246A | YKYYADSVKGRFTISRDNAKNSLYLQMNSLRADDTAVYYCARDLVEPGITPVGCDY | |
| Heavy | WGRGTLVTVSS | |
| NORO- | SNELTQAPSVSVSPGQTARITCFGDALANQYSYWYQRKPGQAPVLVIYKDSERPSGI | 10 |
| 246A | PQRFSASNSGTTVTLTITGVQAEDEADYFCQSADSTGIYKVFGGGTKLTVL | |
| Light | | |
| NORO- | QVQLEVSGGGLVKPGGSLRLSCAASGFTFTDYYMSWIRQAPGKGLEWVAYISGIMS | 11 |
| 327A | STKYADSVKGRFTISRDNGKNSVYLQMNSLTAEDTAVYYCARERVEPPSAVTDFWG | |
| Heavy | QGTLVTVSS | |
| NORO- | QSVLTQSPSVSGAPGQRVTISCTGTSSNIGAGYDVHWYQQFPGTAPKLLISHNTNRP | 12 |
| 327A | SGVPDRFSGSKSGTSASLAITGLQAEDEADYYCQSFDSSLRGSRVFGGGTKLTVL | |
| Light | | |

TABLE 3
CDR HEAVY CHAIN SEQUENCES

| | CDRH1 | CDRH2 | CDRH3 |
|---|---|---|---|
| Antibody | (SEQ ID NO:) | (SEQ ID NO:) | (SEQ ID NO:) |
| NORO-232 | GNSFGAFY | VNYLGGA | ARGRPHDYSPGSYSRPRRYYGLDV |
| | (13) | (14) | (15) |
| NORO-246A | GFTFSRYG | IVIRSDYK | ARDLVEPGITPVGCDY |
| | (16) | (17) | (18) |
| NORO-327A | GFTFTDYY | ISGIMSST | ARERVEPPSAVTDF |
| | (19) | (20) | (21) |

TABLE 4
CDR LIGHT CHAIN SEQUENCES

| | CDRH1 | CDRH2 | CDRH3 |
|---|---|---|---|
| Antibody | (SEQ ID NO:) | (SEQ ID NO:) | (SEQ ID NO:) |
| NORO-232 | QAVSTTY | GTY | QQYSSSPYT |
| | (22) | (23) | (24) |
| NORO-246A | ALANQY | KDS | QSADSTGIYKV |
| | (25) | (26) | (27) |
| NORO-327A | SSNIGAGYD | HNT | QSFDSSLRGSRV |
| | (28) | (29) | (30) |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. References

The following references, to the extent that they provide exemplary procedural or other details supplementary to those set forth herein, are specifically incorporated herein by reference.

### References for Other than Examples 7-9

U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
Atmar et al., Curr Opin Infect Dis., 2018.
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Atherton et al., Biol. of Reproduction, 32, 155-171, 1985.
Barzon et al., Euro Surveill. 2016 Aug. 11; 21(32).
Beltramello et al., Cell Host Microbe 8, 271-283, 2010.
Brown et al., J. Immunol. Meth., 12; 130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
De Jager et al., Semin. Nucl. Med. 23(2), 165-179, 1993.
Dholakia et al., J. Biol. Chem., 264, 20638-20642, 1989.
Diamond et al., J Virol 77, 2578-2586, 2003.
Doolittle and Ben-Zeev, Methods Mol. Biol., 109, :215-237, 1999.
Duffy et al., N. Engl. J. Med. 360, 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet., 3:231-236, 1977.
Gornet et al., Semin Reprod Med. 2016 September; 34(5):285-292. Epub 2016 Sep. 14.
Gulbis and Galand, Hum. Pathol. 24(12), 1271-1285, 1993.
Halfon et al., PLoS ONE 2010; 5 (5) e10569
Hessell et al., Nature 449, 101-4, 2007.
Khatoon et al., Ann. of Neurology, 26, 210-219, 1989.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1):105-132, 1982.
Mansuy et al., Lancet Infect Dis. 2016 October; 16(10):1106-7.
Nakamura et al., In: Enzyme Immunoassays: Heterogeneous and Homogeneous Systems, Chapter 27, 1987.
O'Shannessy et al., J. Immun. Meth., 99, 153-161, 1987.
Persic et al., Gene 187:1, 1997
Potter and Haley, Meth. Enzymol., 91, 613-633, 1983.
Purpura et al., Lancet Infect Dis. 2016 October; 16(10):1107-8. Epub 2016 Sep. 19.
Remington's Pharmaceutical Sciences, 15th Ed., 3:624-652, 1990.
Tang et al., J. Biol. Chem., 271:28324-28330, 1996.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336, 142-151, doi:10.1016/j.jim.2008.04.008, 2008.
Glass et al., N Engl J Med 361:1776-1785, 2009.
Fields et al., Fields Virology. Lippincott Williams & Wilkins; 2013.
Bok & Green, N Engl J Med. 367:2126-2132, 2012.
Vinjé J., J Clin Microbiol 53:373-381, 2015.
Jiang et al., J Virol 66:6527-6532, 1992.
Richardson et al., Expert Rev Vaccines 12:155-167, 2013.
Erdman et al., J Clin Microbiol 27:1417-1418, 1989.
Czako et al., Clin Vaccine Immunol 19:284-287, 2012.
Reeck et al., J Infect Dis 202:1212-1218, 2010.
Jones et al., Science 346:755-759, 2014.
Duizer et al., J Gen Virol 85:79-87, 2004.
Herbst-Kralovetz et al., Emerging Infect Dis 19:431-438, 2013.
Ettayebi et al., Science 353:1387-1393, 2016.
Costantini et al., Emerging Infect Dis 24:1453-1464, 2018.
Sapparapu et al., PLoS Pathog 12:e1005719, 2016.
Loisy et al., J Virol Methods 123:1-7, 2005.
Kageyama et al., J Clin Microbiol 41:1548-1557, 2003.
Green et al., J Clin Microbiol 31:2185-2191, 1993.
Iritani et al., J Med Virol 79:1187-1193, 2007.
Gray et al., J Clin Microbiol 32:3059-3063, 1994.
Atmar et al., Clin Vaccine Immunol 22:923-929, 2015.
Czako et al., Clin Vaccine Immunol 22:221-228, 2015.
Lindesmith et al., PLoS Pathog 8:e1002705, 2012a.
Choi J-M et al., Proc Natl Acad Sci U.S.A. 105:9175-9180, 2008.
Debbink et al., J Virol 86:1214-1226, 2012.
Lindesmith et al., J Virol 86:873-883, 2012b.
Lochridge et al., J Gen Virol 86:2799-2806, 2005.
Parra et al., PLoS ONE 8:e67592, 2013.
Karst & Baric, J Virol 89:5756-5759, 2015.
Huang et al., Protein Eng Des Sel 27:339-349, 2014.
Crawford et al., Clin. Vaccine Immunol 22:168-177, 2015.
Koromyslova et al., ed. PLoS Pathog 13:e1006636, 2017.
Smith & Crowe, Microbiol Spectr 3:AID-0027-2014, 2015.
Debbink et al., J Infect Dis 208:1877-1887, 2013.
Debbink et al., Clin Infect Dis 58:1746-1752, 2014.
El-Kamary et al., J Infect Dis 202:1649-1658, 2010.
Bernstein et al., J Infect Dis 211:870-878, 2015.
Tan et al., J Virol 85:753-764, 2011.
Ramirez et al., Clin Immunol 144:98-108, 2012.
Leroux-Roels et al., J Infect Dis 217:597-607, 2018.
Alvarado et al., Gastroenterology 155(6): 1898-1907, 2018.
Bartsch et al., PLoS ONE 11, e0151219, 2016.
Brochet et al., Nucleic Acids Res. 36, W503-W508, 2008.
Hale et al., J. Medical Virol., 54(4), pp. 305-312, 1998.
Hall et al., A. J., Wikswo, M. E., Pringle, K., Gould, L. H., Parashar, U. D., Division of Viral Diseases, National Center for Immunization and Respiratory Diseases, CDC; United States, 2009-2012. MMWR Morb. Mortal. Wkly. Rep. 63, 491-495, 2014.
Knipe and Howley, Fields Virology (Lippincott Williams & Wilkins), 2013.
Koromyslova et al., J. Virol. 93, 1580, 2019.
Kou et al., Clin. Vaccine Immunol. 22, 160-167, 2015.
Laskowski and Swindells, J. Chem. Inf. Model., 51, 2778-2786. [PubMed id: 21919503], 2011.
McCoy et al., J. Appl. Cryst. 40, 658-674, 2007.
McLean et al., Mol. Immunol. 37, 837-845, 2000.
Parker et al., J. Virol. 79, 7402-7409, 2005.
Parra et al., J. Virol. 86, 7414-7426, 2012.
Pettersen et al., Comput Chem. 25(13):1605-12, 2004.
Pires et al., PLoS ONE 10, e0142927, 2015.
Pletneva et al., Virus Genes 23, 5-16, 2001.
Prasad et al., Science 286, 287-290, 1999.
Shanker et al., Proc Natl Acad Sci USA 113(40):E5830-E5837 (Epub 2016 Sep. 19), 2016.
Shanker et al., J. Virol. 85(17): 8635-8645, 2011.
Tian et al., J. Appl. Microbiol. 109, 1753-1762, 2010.
Treanor et al., J. Clin. Microbiol. 31, 1630-1634, 1993.
Yoda et al., J. Clin. Microbiol. 41, 2367-2371, 2003.
Zheng et al., J. Med. Virol. 90, 671-676, 2018.
Lindesmith, L. et al. Cellular and humoral immunity following Snow Mountain virus challenge. J Virol 79, 2900-2909, doi:10.1128/JVI.79.5.2900-2909.2005 (2005).
Lindesmith, L. C. et al. Sera antibody repertoire analyses reveal mechanisms of broad and pandemic strain neutralizing responses after human norovirus vaccination. Immunity 50, 1530-1541 e1538, doi:10.1016/j.immuni.2019.05.007 (2019).
Prasad, B. V., Hardy, M. E., Jiang, X. & Estes, M. K. Structure of Norwalk virus. Arch Virol Suppl 12, 237-242, doi:10.1007/978-3-7091-6553-9_25 (1996).
Ossiboff, R. J., Zhou, Y., Lightfoot, P. J., Prasad, B. V. & Parker, J. S. Conformational changes in the capsid of a calicivirus upon interaction with its functional receptor. J Virol 84, 5550-5564, doi:10.1128/JVI.02371-09 (2010).
Lindesmith, L. C. et al. Emergence of a norovirus GII.4 strain correlates with changes in evolving blockade epitopes. J Virol 87, 2803-2813, doi:10.11285\71.03106-12 (2013).
Koromyslova, A. D. & Hansman, G. S. Nanobody binding to a conserved epitope promotes norovirus particle disassembly. J Virol 89, 2718-2730, doi:10.1128/JVI.03176-14 (2015).
Song, C. et al. Dynamic rotation of the protruding domain enhances the infectivity of norovirus. PLoS Pathog 16, e1008619, doi:10.1371/journal.ppat.1008619 (2020).
Sherman, M. B. et al. Bile salts alter the mouse norovirus capsid conformation: Possible implications for cell attachment and immune evasion. J Virol 93, doi:10.1128/JVI.00970-19 (2019).
Graziano, V. R. et al. CD3001f is the primary physiologic receptor of murine norovirus but not human norovirus. PLoS Pathog 16, e1008242, doi:10.1371/journal.ppat.1008242 (2020).
Nelson, C. A. et al. Structural basis for murine norovirus engagement of bile acids and the CD3001f receptor. Proc Natl Acad Sci USA 115, E9201-E9210, doi:10.1073/pnas.1805797115 (2018).
Katpally, U., Wobus, C. E., Dryden, K., Virgin, H. W. t. & Smith, T. J. Structure of antibody-neutralized murine norovirus and unexpected differences from viruslike particles. J Virol 82, 2079-2088, doi:10.1128/JVI.02200-07 (2008).
Katpally, U. et al. High-resolution cryo-electron microscopy structures of murine norovirus 1 and rabbit hemorrhagic disease virus reveal marked flexibility in the receptor binding domains. J Virol 84, 5836-5841, doi:10.1128/JVI.00314-10 (2010).
Hansman, G. S. et al. Structural basis for broad detection of genogroup II noroviruses by a monoclonal antibody that binds to a site occluded in the viral particle. J Virol 86, 3635-3646, doi:10.1128/JVI.06868-11 (2012).
Chen, V. B. et al. MolProbity: all-atom structure validation for macromolecular crystallography. Acta Crystallogr D Biol Crystallogr 66, 12-21, doi:10.1107/S0907444909042073 (2010).
Lindesmith, L. C. et al. Mechanisms of GII.4 norovirus persistence in human populations. PLoS Med 5, e31, doi:10.1371/journal.pmed.0050031 (2008).
Otwinowski, Z. & Minor, W. Processing of X-ray diffraction data collected in oscillation mode. Methods Enzymol 276, 307-326 (1997).
Adams, P. D. et al. PHENIX: building new software for automated crystallographic structure determination. Acta Crystallogr D Biol Crystallogr 58, 1948-1954 (2002).
Emsley, P. & Cowtan, K. Coot: model-building tools for molecular graphics. Acta Crystallogr D Biol Crystallogr 60, 2126-2132, doi:10.1107/S0907444904019158 (2004).
Madeira, F. et al. The EMBL-EBI search and sequence analysis tools APIs in 2019. Nucleic Acids Res 47, W636-W641, doi:10.1093/nar/gkz268 (2019).
Atmar, R. L. et al. Comparison of microneutralization and histo-blood group antigen-blocking assays for functional norovirus antibody detection. J Infect Dis 221, 739-743, doi:10.1093/infdis/jiz526 (2020).
Haga, K. et al. Genetic manipulation of human intestinal enteroids demonstrates the necessity of a functional fucosyltransferase 2 gene for secretor-dependent human norovirus infection. mBio 11, doi:10.1128/mBio.00251-20 (2020).
Ettayebi, K. et al. New insights and enhanced human norovirus cultivation in human intestinal enteroids. mSphere 6, doi:10.1128/mSphere.01136-20 (2021).

1. A method of detecting a norovirus infection in a subject comprising:
(a) contacting a sample from said subject with an antibody or antibody fragment having clone-paired heavy and light chain CDR sequences from Tables 3 and 4, respectively; and
(b) detecting norovirus in said sample by binding of said antibody or antibody fragment to a norovirus antigen in said sample.

2-12. (canceled)

13. A method of treating a subject infected with norovirus, or reducing the likelihood of infection of a subject at risk of contracting norovirus, comprising delivering to said subject an antibody or antibody fragment having clone-paired heavy and light chain CDR sequences from Tables 3 and 4, respectively.

14. The method of claim 13, the antibody or antibody fragment is encoded by clone-paired light and heavy chain variable sequences as set forth in Table 1.

15. The method of claim 13, the antibody or antibody fragment is encoded by clone-paired light and heavy chain variable sequences having 95% identity to as set forth in Table 1.

16. The method of claim 13, wherein said antibody or antibody fragment is encoded by light and heavy chain variable sequences having 70%, 80%, or 90% identity to clone-paired sequences from Table 1.

17. The method of claim 13, wherein said antibody or antibody fragment comprises light and heavy chain variable sequences according to clone-paired sequences from Table 2.

18. The method of claim 13, wherein said antibody or antibody fragment comprises light and heavy chain variable sequences having 70%, 80% or 90% identity to clone-paired sequences from Table 2.

19. The method of claim 13, wherein said antibody or antibody fragment comprises light and heavy chain variable sequences having 95% identity to clone-paired sequences from Table 2.

20. The method of claim 13, wherein the antibody fragment is a recombinant scFv (single chain fragment variable) antibody, Fab fragment, F(ab′)2 fragment, or Fv fragment.

21. The method of claim 13, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA-PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

22. The method of claim 13, wherein said antibody is a chimeric antibody or a bispecific antibody.

23. The method of claim 13, wherein said antibody or antibody fragment is administered prior to infection or after infection.

24. The method of claim 13, wherein said subject is a pregnant female, a sexually active female, or a female undergoing fertility treatments.

25. The method of claim 13, wherein delivering comprises antibody or antibody fragment administration, or genetic delivery with an RNA or DNA sequence or vector encoding the antibody or antibody fragment.

26. A monoclonal antibody, wherein the antibody or antibody fragment is characterized by clone-paired heavy and light chain CDR sequences from Tables 3 and 4, respectively.

27-35. (canceled)

36. A hybridoma or engineered cell encoding an antibody or antibody fragment wherein the antibody or antibody fragment is characterized by clone-paired heavy and light chain CDR sequences from Tables 3 and 4, respectively.

37-46. (canceled)

47. A vaccine formulation comprising one or more antibodies or antibody fragments characterized by clone-paired heavy and light chain CDR sequences from Tables 3 and 4, respectively.

48-56. (canceled)

57. A vaccine formulation comprising one or more expression vectors encoding a first antibody or antibody fragment according to claim 26.

58-60. (canceled)

61. A method of protecting the health of a placenta and/or fetus of a pregnant a subject infected with or at risk of infection with norovirus comprising delivering to said subject an antibody or antibody fragment having clone-paired heavy and light chain CDR sequences from Tables 3 and 4, respectively.

62-75. (canceled)

76. A method of determining the antigenic integrity, correct conformation and/or correct sequence of a norovirus antigen comprising:
(a) contacting a sample comprising said antigen with a first antibody or antibody fragment having clone-paired heavy and light chain CDR sequences from Tables 3 and 4, respectively; and
(b) determining antigenic integrity, correct conformation and/or correct sequence of said antigen by detectable binding of said first antibody or antibody fragment to said antigen.

77-96. (canceled)

97. A human monoclonal antibody or antibody fragment, or hybridoma or engineered cell producing the same, wherein said antibody or antibody fragment binds to norovirus capsid protein P domain and/or S domain.

98-101. (canceled)
